(19) United States
(12) Patent Application Publication
(10) Pub. No.: US 2009/0098126 A1
(43) Pub. Date: Apr. 16, 2009

(54) Method and kit for the detection of adenoviruses

(75) Inventors: Karin Ebner, Vienna (AT); Thomas Lion, Vienna (AT)

Correspondence Address: FULBRIGHT & JAWORSKI L.L.P., 600 CONGRESS AVE., SUITE 2400, AUSTIN, TX, 78701, US

(73) Assignees: AUSTRIA WIRTSCHAFTSSERVICE GESELLSCHAFT MIT BESCHRANKTER HAFTUNG; FORSCHUNGSINSTITUT FUR KREBSKRANKE KINDER

(21) Appl. No.: 11/454,158

(22) Filed: Jun. 15, 2006

(51) Int. Cl. A61K 39/395; C12N 15/11; C07K 14/00; C12Q 1/70; A61K 39/00; C07K 16/18

(52) U.S. Cl. 424/139.1; 536/23.72; 530/350; 424/186.1; 530/387.9; 435/5

(57) ABSTRACT

A method for the simultaneous detection of adenoviruses of subgenera A, B, C, D, E and F in a sample comprising contacting said sample with at least one forward primer and at least one reverse primer for a hexon gene of an adenovirus of subgenera A, B, C, D, E and F and subjecting the sample contacted with said primers to a nucleic acid amplification technique.

The Sequence Listing is submitted on one compact disc (Copy 1), together with a duplicate thereof (Copy 2), each created on Jun. 15, 2006, and each containing one 427 kb file entitled “SONNO94.TXT.” The material contained on the compact disc is specifically incorporated herein by reference.

### BACKGROUND OF THE INVENTION

1. Field of the Invention

The present invention relates to a method and a kit for the detection of adenoviruses.

2. Description of Related Art

Adenoviruses (AdV) can cause infections and inflammations in a number of different organs. The most common locations include the gastrointestinal tract, the upper respiratory tract and the eyes(1-3). In individuals with a functional immune system, AdV infections are not associated with life-threatening disease. However, adenoviruses are pathogens causing serious infections in immunosuppressed patients, particularly in HIV-positive individuals and in allogeneic bone marrow transplant recipients, where the infections tend to become invasive, and disseminated disease is associated with very high mortality (4-6). To date, 51 different human AdV serotypes have been identified. On the basis of the oncogenic, hemagglutinating, morphological and DNA sequence properties (3, 13, 16) adenoviruses are divided into six major subgroups (subgenera or species A-F) (6, 7), and all AdV species have be implicated in life-threatening infections in immunosuppressed patients (8). It is therefore of paramount importance for a clinical assay to permit reliable detection of all adenovirus serotypes, whereby in certain clinical situations the detection of each individual serotype may be helpful.

Earlier diagnostic approaches to AdV detection relied mainly on serological tests and cell culture (1, 2, 6, 14, 15). In immunosuppressed patients, however, the use of serological tests is limited due to the impaired immune response, and evaluation of positive cultures is a relatively slow method (25). The recent introduction of PCR-based assays has opened new ways to rapid, specific and highly sensitive AdV detection (9, 17, 18, 19, 25, 24, 28). Many of these diagnostic approaches, however, do not effectively cover all AdV types or use low stringency conditions to permit detection of the genetically highly diverse adenoviruses (12, WO 2004/044238).

The homology of adenovirus DNA sequences between different subgenera is low. Even conserved regions within the AdV genome display only limited homology between adenoviruses from different subgenera. In many instances, considerable differences in DNA sequence even exist between serotypes belonging to the same subgenus. These facts underscore the difficulty to develop molecular tests facilitating reliable screening for AdV infections with the required broad specificity.

A number of molecular assays for AdV detection have been described, relying on the use of the polymerase chain reaction (PCR) or real-time PCR (RQ-PCR). Many of these diagnostic approaches, however, do not effectively cover all AdV types or use low stringency conditions to permit detection of the genetically highly diverse adenoviruses (9-15). Even a recently published assay has permitted detection of a broad spectrum of human adenoviruses-only at low stringency conditions (i.e. a low annealing temperature), thus potentially compromising the specificity and rendering the application of the assay in clinical diagnosis rather problematic (16).

The U.S. Pat. No. 6,541,246 relates to a PCR method for the detection and quantification of adenoviruses in a sample. The primers used in said method are derived from the variable 3′-terminal end of the hexon gene. This method allows the detection of 17 adenovirus serotypes.

The JP 2001 161371 relates to a PCR-based method for the detection of single adenovirus serotypes.

The JP 2004 305092 discloses an RQ-PCR array for the detection of adenoviruses in a sample. However, not all known adenovirus serotypes can be detected by said method.

The JP 7327700 relates to a method for the detection and differentiation of adenoviruses in a sample.

In Avellon A et al. (J Med Virol (2003) 70:228-239) a method for the detection of known adenovirus serotypes involving nested PCR is described.

A PCR method involving primers binding to the hexon gene for the detection of human adenoviruses is disclosed in Serantis H et al. (J Clin Microbiol (2004) 42:3963-3969).

In Takeuchi S et al. (J Clin Microbiol (1999) 37:1839-1845) the determination of the hypervariable regions of the hexon gene of several adenovirus serotypes is described.

The WO 2004/078977 relates to a nucleic acid sequence encoding the adenovirus fiber capsid protein.

In the WO 01/59163 a method for the identification and/or quantification of nucleic acids of adenoviruses using primers derived from nucleotides 21000 to 22000 of the adenovirus serotype 5 genome is disclosed.

Scott-Taylor T H et al. (J Clin Microbiol (1992) 30:1703-1710) describe the use of conserved regions of the adenovirus genome, in particular of the hexon gene of adenovirus serotype 2, for the detection of human adenoviruses.

In Wan Hong X et al. (J Clin Microbiol (2001) 38:4114-4120) a multiplex PCR assay involving primers binding to the fiber gene of adenoviruses for their detection is described.

### SUMMARY OF THE INVENTION

It is an object of the present invention to provide a method, a kit and means for reliable, highly specific and sensitive detection of AdV, which may optionally be performed simultaneously for all known human AdV serotypes, whereby the assay may be completed within a few hours, starting from sample collection to the report of results. Furthermore the task of detecting distinct adenovirus subgenera or serotypes should be performable in a very easy way, employing only a very limited number of working steps.

Therefore, the present invention relates to a method for the simultaneous detection of adenoviruses of subgenera A, B, C, D, E and F in a sample comprising the steps:

a) providing a sample,

b) contacting said sample with at least one forward primer and at least one reverse primer having at least 80% identity with a portion of a nucleotide sequence of a hexon gene of an adenovirus of subgenera A, B, C, D, E and F and being derived from sequences SEQ ID No. 1 and/or SEQ ID No. 2 and/or SEQ ID No. 3 and/or SEQ ID No. 4 to 8 and/or SEQ ID No. 10 to 12 and preferably at least one forward primer and at least one reverse primer having at least 80% identity with a portion of a fiber gene of at least one adenovirus of subgenera A, B, C, D, E and/or F,

c) subjecting the sample contacted with said primers to a nucleic acid amplification technique, and

d) optionally determining the presence of adenoviruses in said sample by detecting a nucleic acid amplification product.

The method provided herein shows several advantages over currently known assays including substantially lower costs with regard to reagents and labour (one or two nucleic acid amplification steps needed), while the broad specificity and sensitivity have been maintained. The test permits rapid quantitative detection of all known adenovirus serotypes, which is an important prerequisite for timely onset of appropriate treatment. Wide application of the assay in clinical virus screening may therefore contribute to an improvement of the outcome of adenovirus infections in immunocompromised patients. This method can also be used in high throughput screenings. Especially when combining primers hybridising to a hexon gene with primers hybridising to a fiber gene results in an even more powerful tool. In contrast thereto, known diagnostic approaches do not effectively cover all AdV types or use low stringency conditions to permit detection of the genetically highly diverse adenoviruses. Also a five-reaction real-time PCR assay recently introduced permitting the detection and the quantification of all 51 currently known human AdV serotypes (21) using five single amplification steps is due to high costs and labour associated not suited for the use in the routine diagnostic setting.

“Simultaneous detection” means, that all known AdV members of the subgenera groups can be detected by a single or maximum two or three nucleic acid amplification steps.

According to the present invention “nucleic acid” refers to DNA as well as to RNA and mRNA.

The term “sample” includes all type of samples which may be infected by an adenovirus and comprises therefore, especially clinical samples (e.g. blood serum, tissues) of, for instance, immunosuppressed patients. The sample may be prepared before contacting it to the primers by standard laboratory methods (e.g. homogenisation and/or fractionation of the sample, purification or precipitation of the nucleic acid) or used directly for the nucleic acid amplification.

The terms “homology” and “identity”, as defined herein, are often used interchangeably. In general, sequences are aligned so that the highest order match is obtained (see, e.g.: Computational Molecular Biology, Lesk, A. M. , ed., Oxford University Press, New York, 1988 Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073).

By sequence identity, the number of conserved amino acids or nucleic acids is determined by standard alignment algorithms programs, and is used with default gap penalties established by each supplier. Substantially homologous nucleic acid molecules would hybridize typically at moderate stringency or at high stringency all along the length of the nucleic acid or along at least about 70%, 80% or 90% of the full-length nucleic acid molecule of interest. Also contemplated are nucleic acid molecules that contain degenerate codons in place of codons in the hybridizing nucleic acid molecule.

Whether any two nucleic acid molecules have nucleotide sequences that are at least, for example, 80%, 85%, 90%, 95%, 96%, 97%, 98% or 99% “identical” can be determined using known computer algorithms such as the “FAST A” program, using for example, the default parameters as in Pearson et al. (1988) PNAS USA 85: 2444 (other programs include the GCG program package (Devereux, J., et al., Nucleic Acids Research (1984) Nucleic Acids Res., 12, 387-395), BLASTP, BLASTN, FASTA (Atschul, S. F., et al., J Molec Biol 215: 403 (1990); Guide to Huge Computers, Martin J. Bishop, ed., Academic Press, San Diego, 1994, and Carilloet al, (1988)SIAM J Applied Math 48 : 1073). For instance, the BLAST tool of the NCBI database can be used to determine identity. Other commercially or publicly available programs include, DNAStar “MegAlign” program (Madison, Wis.) and the University of Wisconsin Genetics Computer Group(UWG) “Gap” program (Madison Wis.)). Percent homology or identity of proteins and/or nucleic acid molecules can be determined, for example, by comparing sequence information using a GAP computer program (e.g. Needleman et al., (1970) J. Mol. Biol. 48:443, as revised by Smith and Waterman (1981) Adv. Appl. Math. 2:482). Briefly, the GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and for non-identities) and the weighted comparison matrix of Gribskov et al. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

As used herein, the term “at least 80% identical to” refers to percent identities from 80 to 99.99 relative to the reference polypeptides.

Identity at a level of 80% or more is indicative of the fact that, assuming for exemplification purposes a test and reference polynucleotide length of 100 nucleic acids are compared, no more than 20% (i.e. 20 out of 100) of nucleic acids in the test polynucleotide differs from that of the reference polynucleotide. Similar comparisons can be made between a test and reference polypeptides. Such differences can be represented as point mutations randomly distributed over the entire length of an nucleic/amino acid sequence or they can be clustered in one or more locations of varying length up to the maximum allowable, e.g. 20/100 amino acid difference (approx. 80% identity). Differences are defined as nucleic acid or amino acid substitutions, or deletions. At the level of homologies or identities above about 85-90%, the result should be independent of the program and gap parameters set; such high levels of identity can be assessed readily, often without relying on software.

Stringency of hybridization in determining percentage mismatch is as follows:

1) high stringency: 0.1×SSPE, 0.1% SDS, 65° C.

2) medium stringency: 0.2×SSPE, 0.1% SDS, 50° C.

3) low stringency: 1.0×SSPE, 0.1% SDS, 50° C.

The person skilled in the art knows that the washing step selects for stable hybrids and also know the ingredients of SSPE (see e.g. Sambrook, E. F. Fritsch, T. Maniatis, in: Molecular Cloning, A Laboratory Manual, Cold Spring Harbor Laboratory Press (1989), vol. 3, see, also, numerous catalogs that describe commonly used laboratory solutions). SSPE is pH 7.4 phosphate-buffered 0.18 M NaCl. Further, those of skill in the art recognize that the stability of hybrids is determined by the melting temperature (Tm), which is a function of the sodium ion concentration and temperature (Tm=81.5° C.+16.6 (log [Na+])+0.41 (% G+C)−(600/length)), so that the only parameters in the wash conditions critical to hybrid stability are sodium ion concentration in the SSPE (or SSC) and temperature.

It is understood that equivalent stringencies can be achieved using alternative buffers, salts and temperatures. By way of example and not limitation, procedures using conditions of low stringency are as follows (see also Shilo and Weinberg, PNAS USA 78: 6789-6792 (1981)): Filters containing DNA are pretreated for 6 hours at 40° C. in a solution containing 35% formamide, 5×SSC, 50 mM Tris-HCI (pH 7.5), 5 mM EDTA, 0.1% PVP, 0.1% Ficoll, 1% BSA, and 500 μg/ml denatured salmon sperm DNA (10×SSC is 1.5 M sodium chloride, and 0.15 M sodium citrate, adjusted to a pH of 7).

Hybridizations are carried out in the same solution with the following modifications: 0.02% PVP, 0.02% Ficoll, 0.2% BSA, 100, μg/ml salmon sperm DNA, 10% (wt/vol) dextran sulfate, and 5-20×10⁶ cpm 32P-labeled probe is used. Filters are incubated in hybridization mixture for 18-20 hours at 40° C., and then washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours at 60° C. Filters are blotted dry and exposed for autoradiography. If necessary, filters are washed for a third time at 65-68° C. and re-exposed to film. Other conditions of low stringency which can be used are well known in the art (e.g. as employed for cross-species hybridizations).

By way of example and not way of limitation, procedures using conditions of moderate stringency include, for example, but are not limited to, procedures using such conditions of moderate stringency are as follows: Filters containing DNA are pre-treated for 6 hours at 55° C. in a solution containing 6×SSC, 5×Denhart's solution, 0.5% SDS and 100 μg/ml denatured salmon sperm DNA. Hybridizations are carried out in the same solution and 5-20×10⁶ cpm 32P-labeled probe is used. Filters are incubated in hybridization mixture for 18-20 hours at 55° C., and then washed twice for 30 minutes at 60° C. in a solution containing 1×SSC and 0.1% SDS. Filters are blotted dry and exposed for autoradiography.

Other conditions of moderate stringency which can be used are well-known in the art. Washing of filters is done at 37° C. for 1 hour in a solution containing 2×SSC, 0.1% SDS.

By way of example and not way of limitation, procedures using conditions of high stringency are as follows: pre-hybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in pre-hybridization mixture containing 100 μg/ml denatured salmon sperm DNA and 5-20×10⁶ cpm of 32P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 minutes before autoradiography. Other conditions of high stringency which can be used are well known in the art.

A primer having e.g. 80% of identity with a nucleotide sequence means that not only the composition of the primers is 80% identical with the nucleotide sequences but also their sequential order.

“Being derived from sequences” means that the primers of the present invention are directly deduced from the nucleotide sequences (alignments and individual sequences) disclosed herein (SEQ ID No. 1 to 12 and 31 to 65) and are at least 80%, at least 90%, at least 95%, at least 98%, at least 99% or 100% identical to them. These primers can be obtained by simple chemical synthesis (e.g. Caruthers, M. H. (1983) Tetrahedron Lett. 24:245-248) or by enzymatic or chemical cleavage of a nucleic acid sequence comprising a primer sequence.

The sequences SEQ ID No. 1, SEQ ID No. 2 and SEQ ID No. 3 are consensus sequences of the conserved regions of the hexon genes of all known adenovirus serotypes (currently 51; variable regions are not suited to obtain primers which allow the simultaneous detection of AdVs of at least two subgenera). These consensus sequences (including SEQ ID No. 31 to 65, AdV 1:AF534906 (NCBI Acc. No.), AdV 2:AJ293905, AdV 3:X76549, AdV 4:X84646, AdV 5:X02997, AdV 7:AF515814, AdV 11:AF532578, AdV 12:NC_{—}001460, AdV 16:X74662, AdV 17:BK000406, AdV 21:AY008279, AdV 34:AB052911, AdV 35:AB052912, AdV 40: X51782, AdV 41:D13781, AdV 41:U20821) are obtained by the alignment of genes encoding for the hexon protein following the blossom 62-12-2 sample comparison table. Of course also other sample comparison tables may be used for the identification of such a consensus sequence. A consensus sequence allows the person skilled in the art to identify highly conserved regions within the nucleotide sequences aligned. If highly conserved regions are identified, primers derived from these regions (on the forward or on the complementary strand) can be determined. Of course also an alignment of the hexon protein sequences may be used to identify identical regions or regions with a high homology which may serve for the determination of primer pairs (including degenerated primers). The nucleic acid sequences SEQ ID No. 4 to 6, 7 and 8 and 10 to 12 are alignments of AdV hexon genes of subgenera A and C and B, D, E, respectively.

The term “at least one forward primer and at least one reverse primer” refers to the fact that according to the present invention one ore more forward or reverse primers may be employed in a single or in a multiple series of a nucleic acid amplification technique. Some of these techniques, e.g. nested PCR, TaqMan PCR, may employ more than one forward and more than one reverse primer. The nucleic acid sequences for the production of the alignment are taken from the NCBI or EMBL databases.

The method according to the present invention may further involve a second primer pair being in a certain degree identical with a portion of a fiber gene of an adenovirus (AdV 40, fiber-2: Acc. No. L19443). An alignment of the fiber-2 genes of AdV subgenus F (AdV serotypes 40 and 41) is disclosed herein (SEQ ID No. 9).

The primers used in this method may have at least 80%, at least 85%, at least 90%, at least 95%, at least 98%, at least 99% or 100% identity with a portion of their corresponding nucleotide sequence strand. In fact the primers can also be completely identical to a portion of a hexon gene and/or fiber gene. If this is the case these primers may be used under optimised conditions to detect specifically AdV of a distinct serotype or AdV of at least one subgenus.

The selection of primers having at least 80% identity with the portion of a hexon or fiber gene of all known adenovirus serotypes allows not only the simple detection of one single adenovirus serotype but enables the person skilled in the art to simultaneously detect several adenoviruses of subgenera A, B, C, D, E and F. The specification of the consensus sequences of the hexon genes allows also the combined detection of adenoviruses of subgenera A to F in any possible combination. For instance if a person skilled in the art identifies specific primer pairs present in portions of the hexon gene of adenoviruses of subgenera A and E, the specific identification of members of these groups is possible. Optionally to improve the selectivity and sensitivity of the detection it is also possible to combine the primer pairs for the amplification of the hexon gene with primer pairs of other genes, for example of a fiber gene.

Typically primers deduced from a nucleic acid sequence (alignment sequence or single sequence) may be designed following general rules known to the person skilled in the art (e.g. Dieffenbach, C. W., et al., General Concepts for PCR Primer Design, in PCR Primer, A Laboratory Manual, Dieffenbach, C. W., and Dveksler, G. S., Ed., Cold Spring Harbor Laboratory Press, New York, 1995, 133-155; Innis, M. A., and Gelfand, D. H., Optimization of PCRs, in PCR protocols, A Guide to Methods and Applications, Innis, M. A., Gelfand, D. H., Sninsky, J. J., and White, T. J., Ed., CRC Press, London, 1994, 5-11; Sharrocks, A. D., The design of primers for PCR, in PCR Technology, Current Innovations, Griffin, H. G., and Griffin, A. M, Ed., CRC Press, London, 1994, 5-11; Suggs, S. V., et al., Using Purified Genes, in ICN-UCLA Symp. Developmental Biology, Vol. 23, Brown, D. D. Ed., Academic Press, New York, 1981, 683; Kwok, S., et al., Effects of primer-template mismatches on the polymerase chain reaction: Human Immunodeficiency Virus 1 model studies. Nucleic Acids Res. 18:999-1005, 1990). The primers employed in a method according to present invention may be partly degenerated or contain no degenerated nucleotide position. Therefore suited regions on the nucleic acid sequences may be selected to design primers with a low number of degenerated nucleotide positions, wherein suited primers comprise less than 80%, less than 70%, less than 60%, less than 50%, less than 40%, less than 30%, less than 20%, less than 15%, less than 10%, less than 5%, or no degenerated nucleotide positions. Instead of nucleotides in the degenerated nucleotide positions, inosine may be used. However, primers without any degenerated nucleotide position are preferably used.

Furthermore, the present invention relates to a method for the simultaneous detection of adenoviruses of subgenera A, C and F in a sample comprising the steps:

a) providing a sample containing a nucleic acid,

b) contacting said sample with at least one forward primer and at least one reverse primer having at least 80% identity with a portion of a nucleotide sequence of a hexon gene of an adenovirus of subgenera A, C and F and being derived from sequences SEQ ID No. 1 and/or SEQ ID No. 2 and/or SEQ ID No. 3 and/or SEQ ID No. 4 to 8 and preferably at least one forward primer and at least one reverse primer having at least 80% identity with a portion of a fiber gene of at least one adenovirus of subgenera A, C and/or F and being derived from sequences SEQ ID No. 9,

c) subjecting the sample contacted with said primers to a nucleic acid amplification technique, and

d) optionally determining the presence of adenoviruses in said sample by detecting a nucleic acid amplification product and to a method for the simultaneous detection of adenoviruses of subgenera B, D and E in a sample comprising the steps:

a) providing a sample containing a nucleic acid,

b) contacting said sample with at least one forward primer and at least one reverse primer having at least 80% identity with a portion of a nucleotide sequence of a hexon gene of an adenovirus of subgenera B, D and E and being derived from sequences SEQ ID No. 1 and/or SEQ ID No. 2 and/or SEQ ID No. 3 and/or SEQ ID No. 10 to 12 and optionally at least one forward primer and at least one reverse primer having at least 80% identity with a portion of a fiber gene of at least one adenovirus of subgenera B, D and/or E,

c) subjecting the sample contacted with said primers to a nucleic acid amplification technique, and

d) optionally determining the presence of adenoviruses in said sample by detecting a nucleic acid amplification product.

The simultaneous detection of the adenoviruses of subgenera A, C and F and B, D and E allows the detection of all known adenoviruses in one, in two, in three or in four single nucleic acid amplification steps.

According to the present invention the primers chosen may also be positioned separately on three highly homologous sequences such as e.g. SEQ ID No. 1 to 3. For instance, a forward primer may hybridise to the nucleotide sequence SEQ ID No.1, whereas the reverse primer may hybridise on the complementary strand of the nucleotide sequences SEQ ID No. 2 or 3. If nucleic acid amplifications are performed with such primer pairs the length of the fragments obtained may also be a hint which adenovirus is detected, because the distance of the non-homologous sequences in between e.g. SEQ ID No. 1 and 2 may vary depending of the adenovirus serotype. Of course a nucleic acid amplification, wherein the forward primer and the reverse primer hybridise in a close distance (e.g. minimum of 25, of 40, of 60, of 80, of 100, of 150, of 200, of 300, nucleotides) on the hexon genes has practical advantages because the time required for the amplification of the product will be reduced.

The nucleic acid sequences SEQ ID No. 4 to 6 and 7, 8 are alignments of conserved portions of known hexon genes of members of the AdV subgenus A and C, respectively, and nucleic acid sequence SEQ ID No. 9 of known fiber-2 genes of members of the AdV subgenus F. However, also fiber-2 genes of AdV of other subgenera may be used for alignment and consequently to design appropriate primers. The amino and nucleic acid sequences may be obtained from public databases (e.g. NCBI, EMBL).

According to a preferred embodiment the primers have at least 80% identity with a portion of the hexon gene which consists of nucleotides 1 to 392, 1 to 70, 30 to 100, 50 to 120, 70 to 140, 90 to 160, 110 to 180, 130 to 200, 150 to 220, 170 to 240, 190 to 260, 210 to 280, 230 to 300, 250 to 320, 270 to 340, 290 to 360, 310 to 380 or 322 to 392, of SEQ ID No. 1 and/or SEQ ID No. 4 and/or SEQ ID No. 7 and/or SEQ ID No. 10.

The portion of the hexon gene from which the primers are derived from preferably consists of nucleotides 1 to 278, 1 to 70, 30 to 100, 50 to 120, 70 to 140, 90 to 160, 110 to 180, 130 to 200, 150 to 220, 170 to 240, 190 to 260 or 208 to 278, of SEQ ID No. 2 and/or SEQ ID No. 5 and/or SEQ ID No. 11.

According to another preferred embodiment the portion of the hexon gene consists of nucleotides 1 to 1498, 1 to 70, 30 to 100, 50 to 120, 70 to 140, 90 to 160, 110 to 180, 130 to 200, 150 to 220, 170 to 240, 190 to 260, 210 to 280, 230 to 300, 250 to 320, 270 to 340, 290 to 360, 310 to 380, 330 to 400, 350 to 420, 370 to 440, 390 to 460, 410 to 480, 430 to 500, 450 to 520, 470 to 540, 490 to 560, 510 to 580, 530 to 600, 550 to 620, 570 to 640, 590 to 660, 610 to 680, 630 to 700, 650 to 720, 670 to 740, 690 to 760, 710 to 780, 730 to 800, 750 to 820, 770 to 840, 790 to 860, 810 to 880, 830 to 900, 850 to 920, 870 to 940, 890 to 960, 910 to 980, 930 to 1000, 950 to 1020, 970 to 1040, 990 to 1060, 1010 to 1080, 1030 to 1100, 1050 to 1120, 1070 to 1140, 1090 to 1160, 1110 to 1180, 1130 to 1200, 1150 to 1220, 1170 to 1240, 1190 to 1260, 1210 to 1280, 1230 to 1300, 1250 to 1320, 1270 to 1340, 1290 to 1360, 1310 to 1380, 1330 to 1400, 1350 to 1420, 1370 to 1440, 1390 to 1460, 1410 to 1480 or 1428 to 1498, of SEQ ID No. 3 and/or SEQ ID No. 6 and/or SEQ ID No. 8 and/or SEQ ID No. 12.

The use of primers which show a high degree of identity with the portions of SEQ ID No. 1 to 8 and SEQ ID No. 10 to 12 as disclosed above, turned out to be suited for the simultaneous detection of all adenoviruses or of defined adenovirus groups (e.g. subgenera A, C and F, subgenera B, D and E).

The portion of the fiber gene consists preferably of nucleotides 1000 to 1600 of SEQ ID No. 9 (AdV 40/AdV 41, fiber-2: Acc. No. L19443).

The nucleic acid sequence of a fiber gene can simply be taken from a public database.

According to another preferred embodiment said portion of the fiber gene consists of nucleotides 1200 to 1500.

The at least one forward primer and at least one reverse primer consist of 14 to 70, preferably of 16 to 50, especially of 18 to 40 nucleotides.

It is a crucial point of every nucleic acid amplification method that the length of the primers is chosen accurately. If for instance the primers are too long, there is a strong risk the primers form hair pins, loops or dimers. All these effects have mainly a negative impact on sensitivity of the nucleic acid amplification. Furthermore, too short primers do not contribute to an increase in sensitivity and need much lower annealing temperatures.

According to a preferred embodiment the at least one forward primer having at least 80% identity with a portion of a hexon gene is selected from the group consisting of 5′-ggkctggtgcaattcgcc-3′ (SEQ ID No. 13), 5′-acctgggccaaaaccttctc-3′ (SEQ ID No. 14), 5′-acatgcacatcgccgg-3′ (SEQ ID No. 15), 5′-tcgatgatgccgcagtg-3′ (SEQ ID No. 16) and combinations thereof, whereby the primers SEQ ID No. 13, 14 and 16 and SEQ ID No. 15 may be employed in a method for the detection of AdV serotypes A, C and/or F and serotypes B, D and/or E, respectively.

Preferably the at least one reverse primer having at least 80% identity with a portion of a hexon gene is selected from the group consisting of 5′-cacgggcacaaaacgca-3′ (SEQ ID No. 17), 5′-cgtccatgggatccacctc-3′ (SEQ ID No. 18), 5′-cggtcsgtggtcacatc-3′ (SEQ ID No. 19), 5′-gagggtgaagtaggtgtc-3′ (SEQ ID No. 20), 5′-caggctgaagtaggtatc-3′ (SEQ ID No. 21) and combinations thereof, wherein the primers SEQ ID No. 17, 18, 20 and 21 and SEQ ID No. 19 may be preferably employed in a method for the detection of AdV serotypes A, C and/or F and serotypes B, D and/or E, respectively.

According to a preferred embodiment the at least one forward primer having at least 80% identity with a portion of a fiber gene is selected from the group consisting of 5′-cccgtgtttgacaacgaagg-3′ (SEQ ID No. 22). This primer is derived from SEQ ID No. 9 (alignment fiber-2 gene of AdV serotypes 40 and 41).

Preferably the at least one reverse primer having at least 80% identity with a portion of a fiber gene is selected from the group consisting of 5′-ttagagctaggcataaattctacagca-3′ (SEQ ID No. 23).

The nucleic acid amplification technique is preferably a polymerase chain reaction technique which is selected from the group consisting of real-time PCR, preferably TaqMan PCR, quantitative PCR, nested PCR, asymmetric PCR, multiplex PCR, inverse PCR, rapid PCR and combinations thereof. Due to the specificity of these polymerase chain reaction techniques all of them are suited to detect the presence of at least one adenovirus in a sample when using appropriate primers as described above. However, in practice the use of e.g. real-time PCR leads to faster results.

Preferably the nucleic acid amplification technique is performed with at least one oligonucleotide probe hybridising in between the at least one forward primer and the at least one reverse primer within the hexon gene and/or fiber gene of an adenovirus. Also this olignucleotide shares at least 80%, at least 90%, at least 95%, at least 98%, at least 99% or 100% identity with a nucleic acid sequence SEQ ID No. 1 to 12.

“Hybridising” is the ability of primers consisting of nucleic acids to bind specifically to a nucleic acid sequence under stringent conditions and under conditions applied in the course of a polymerase chain reaction. Stringent conditions are sequence-dependent and will be different in different circumstances. Longer sequences hybridise specifically at higher temperatures. An extensive guide to the hybridisation of nucleic acids is found in Tijssen, Techniques in Biochemistry and Molecular Biology-Hybridisation with Nucleic Probes, “Overview of principles of hybridisation and the strategy of nucleic acid assays” (1993). Generally, stringent conditions are selected to be about 5 to 10° C. lower than the thermal melting point (Tm) for the specific sequence at a defined ionic strength and pH. The Tm is the temperature (under defined ionic strength, pH, and nucleic acid concentration) at which 50% of the probes complementary to the target hybridise to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides).

According to another preferred embodiment the at least one oligonucleotide probe is tagged with a dye, preferably a fluorescent dye and optionally with a quencher. An additional oligonucleotide, which hybridises between the at least one forward and at least one reverse primer within the hexon genes or its consensus sequence or the fiber gene, is tagged with a dye and a quencher (e.g. FAM/TAMRA). If added to the PCR reaction mixture, the nucleic acid fragment may be visualised already during the synthesis in a real-time PCR (quantitative PCR). Furthermore, the addition of another oligonucleotide enhances the specificity and yield of a PCR.

Preferably the at least one oligonucleotide probe hybridising to the hexon gene comprises the sequence 5′-ccacggacacctacttcaccctggg-3′ (SEQ ID No. 24), 5′-aactccgcccacgcgctaga-3′ (SEQ ID No. 25), 5′-cgggtctggtgcagt-3′ (SEQ ID No. 26), 5′-cgggtttggtgcagt-3′ (SEQ ID No. 27), 5′-AGGACGCCTCGGAGT-3′ (SEQ ID No. 28) and/or 5′-TCAGGATGCCTCGGA-3′ (SEQ ID No. 29) and the at least one oligonucleotide probe hybridising to the fiber gene comprises the sequence 5′-atcgacaaggacagtctgccaacactaacg-3′ (SEQ ID No. 30), wherein the oligonucleotides SEQ ID No. 24, 25, 28, 29 and 30 and SEQ ID No. 26 and 27 may be preferably employed in a method for the detection of AdV serotypes A, C and/or F and serotypes B, D and/or E, respectively.

According to a preferred embodiment of the present invention the amplified nucleic acid product is additionally tagged for detection by a technique selected from the group consisting of DNA-tagging by random-priming, DNA-tagging by nick-translation, DNA-tagging by polymerase chain reaction, oligonucleotide tailing, hybridisation, tagging by kinase activity, fill-in reaction applying Klenov fragment, photobiotinylation and combinations thereof. Due to a tagging of the PCR product, this product can easily be visualised by an additional further step involving for instance gel electrophoresis.

Preferably the amplified nucleic acid product is detected by gel electrophoresis, Southern-blot, photometry, chromatography, colorimetry, fluorography, chemiluminscence, autoradiography, detection by specific antibody and combinations thereof.

Another aspect of the present invention relates to a kit for the simultaneous detection of adenoviruses of subgenera A, B, C, D, E and F in a sample comprising at least one forward primer and at least one reverse primer having at least 80% identity with a portion of a nucleotide sequence of a hexon gene of an adenovirus of subgenera A, B, C, D, E and F and being derived from sequences SEQ ID No. 1 and/or SEQ ID No. 2 and/or SEQ ID No. 3 and/or SEQ ID No. 4 to 8 and/or SEQ ID No. 10 to 12 and preferably at least one forward primer and at least one reverse primer having at least 80% identity with a portion of a fiber gene of at least one adenovirus of subgenera A, B, C, D, E and/or F and a kit for the simultaneous detection of adenoviruses of subgenera A, C and F in a sample comprising at least one forward primer and at least one reverse primer having at least 80% identity with a portion of a nucleotide sequence of a hexon gene of an adenovirus of subgenera A, C and F and being derived from sequences SEQ ID No. 1 and/or SEQ ID No. 2 and/or SEQ ID No. 3 and/or SEQ ID No. 4 to 8 and preferably at least one forward primer and at least one reverse primer having at least 80% identity with a portion of a fiber gene of at least one adenovirus of subgenera A, C and/or F and being derived from sequences SEQ ID No. 9 and a kit for the simultaneous detection of adenoviruses of subgenera B, D and E in a sample comprising at least one forward primer and at least one reverse primer having at least 80% identity with a portion of a nucleotide sequence of a hexon gene of an adenovirus of subgenera B, D and E and being derived from sequences SEQ ID No. 1 and/or SEQ ID No. 2 and/or SEQ ID No. 3 and/or SEQ ID No. 10 to 12 and optionally at least one forward primer and at least one reverse primer having at least 80% identity with a portion of a fiber gene of at least one adenovirus of subgenera B, D and/or E.

According to further embodiments of the present invention the kit comprises all components which have been already disclosed for the method according to the present invention.

Another aspect for the present invention relates to the use of a kit according to the present invention in a method according to the present invention.

Another aspect of the present invention relates to a nucleic acid molecule encoding an antigen or a fragment there of comprising a nucleic acid sequence with at least 15 nucleotides having at least 80% sequence identity to at least a part of a nucleic acid molecule selected from SEQ ID No. 31 to 65.

The identification of new sequences facilitates the characterization of epitopes relevant for the recognition of individual adenovirus serotypes by the immune system, thus enabling the design of tailored treatment approaches, for example by cytotoxic T lymphocytes (29). Moreover, the comprehensive information on hexon gene sequences from all adenovirus serotypes can serve as a basis for gene therapy-related treatment approaches, such as the RNA interference or antisense RNA techniques (30-32).

A nucleic acid molecule coding for an antigen and cloned into a vector may be used to recombinantly express said antigen in a prokaryotic or eukaryotic host. Several methods, experimental and computational, are known to the person skilled in the art for the identification of said nucleic acid sequences.

According to a preferred embodiment the sequence identity is at least 90%, preferably at least 95%, especially 100%.

Preferably the nucleic acid is DNA, RNA or mRNA.

According to a preferred embodiment of the present invention the nucleic acid molecule is isolated from viral DNA, especially from a Adenovirus DNA, or synthesised chemically.

Depending on the length of the nucleic acid molecule an adequate method to produce said molecule has to be chosen by the person skilled in the art. Small molecules having a maximum of 100, 80, 60, 50 or 40 nucleotides may be synthesised chemically. Fragments may be obtained also directly from the viral adenovirus DNA by e.g. fragmenting the viral DNA by chemical, mechanical or enzymatic methods or by nucleic acid amplification (e.g. polymerase chain reaction).

The fragments obtained by the above mentioned methods may be cloned into a vector, which is capable to express when introduced in a eukaryotic or prokaryotic host, the antigen encoded by the nucleic acid fragment. Suitable vectors are known to the person skilled in the art and can be obtained commercially.

Another aspect of the present invention relates to an antigen comprising an amino acid sequence being encoded by a nucleic acid molecule according to the present invention and fragments thereof, wherein the amino acid sequence is elected from the group consisting of amino acid sequences SEQ ID No. 66 to 100.

Preferably the antigen comprises amino acids 1 to 36 of SEQ ID No. 66 to 100 (AdV serotypes 6, 8-10, 13-15, 18-20, 22-33, 36-39, 42-47, 49-51), amino acids 46 to 69 of SEQ ID No. 66 to 75 or 77 to 100 (AdV serotypes 6, 8-10, 13-15, 18-20, 23-33, 36-39, 42-47, 49-51), amino acids 81 to 92 of SEQ ID No. 66 to 75, 79, 81 to 94 or 98 to 100 (AdV serotypes 6, 8-10, 13-15, 18-20, 25, 27-33, 36-39, 42-44, 49-51), amino acids 102 to 225 of SEQ ID No. 66 to 68, 70 to 75, 79, 81 to 94 or 98 to 100 (AdV serotypes 6, 8, 9, 13-15, 18-20, 25, 27-33, 36-39, 42-44, 49-51), amino acids 235 to 286 of SEQ ID No. 66 to 75, 79, 81 to 94 or 98 to 100 (AdV serotypes 6, 8-10, 13-15, 18-20, 25, 27-33, 36-39, 42-44, 49-51), amino acids 327 to 368 of SEQ ID No. 66 to 68, 70 to 75, 79, 81 to 94 or 98 to 100 (AdV serotypes 6, 8, 9, 13-15, 18-20, 25, 27-33, 36-39, 42-44, 49-51), amino acids 378 to 392 of SEQ ID No. 66 to 68, 70 to 75, 79, 81 to 94 or 98 to 100 (AdV serotypes 6, 8, 9, 13-15, 18-20, 25, 27-33, 36-39, 42-44, 49-51), amino acids 414 to 472 of SEQ ID No. 66 to 68, 70 to 75, 79, 81 to 94 or 98 to 100 (AdV serotypes 6, 8, 9, 13-15, 18-20, 25, 27-33, 36-39, 42-44, 49-51), amino acids 488 to 520 of SEQ ID No. 66 to 68, 70 to 75, 79, 81 to 94 or 98 to 100 (AdV serotypes 6, 8, 9, 13-15, 18-20, 25, 27-33, 36-39, 42-44, 49-51), amino acids 536 to 572 of SEQ ID No. 66 to 76, 78 to 100 (AdV serotypes 6, 8, 9, 10, 13-15, 18-20, 22, 24-33, 36-39, 42-47, 49-51), amino acids 582 to 637 of SEQ ID No. 67, 69 to 81, 83 to 88 or 90 to 100 (AdV serotypes 8, 10, 13-15, 18-20, 22-27, 29-33, 36, 38, 39, 42-47, 49-51), amino acids 647 to 698 of SEQ ID No. 67, 69 to 72, 74 to 81, 83 to 88 or 90 to 100 (AdV serotypes 8, 10, 13-15, 19-20, 22-27, 29-33, 36, 38, 39, 42-47, 49-51), amino acids 708 to 885 of SEQ ID No. 69 to 72, 74 to 81, 83, 84, 87, 88, 90 to 98 or 100 (AdV serotypes 10, 13-15, 19-20, 22-27, 29-30, 32, 33, 36, 38, 39, 42-47, 49, 51), amino acids 895 to 903 of SEQ ID No. 69 to 72, 74 to 81, 83, 84, 87, 88, 90 to 98 or 100 (AdV serotypes 10, 13-15, 19-20, 22-27, 29-30, 32, 33, 36, 38, 39, 42-47, 49, 51).

Another aspect of the present invention relates to the use of an antigen or nucleic acid (e.g. siRNA, antisense RNA) according to the present invention for the manufacture of a medicament for the treatment and/or the prevention of a viral adenovirus infection.

The purified antigen may be used in an appropriate vaccine formulation for the active immunisation of an individual. To enhance the immune response to said antigens, these may be administered with adjuvants such as Alum. The use of antigens or antibodies directed to antigens of a distinct AdV serotype or subgenus for the immunisation or the treatment of a patient, preferably an immunosuppressed patient, allows the targeted therapy of said individual, because in general AdV infected individuals are seized with a low number of different AdV serotypes (maximum 10, maximum 8, maximum 6, maximum 4, maximum 2, maximum 1 AdV serotype). The AdV serotype or subgenera may be determined by a method according to the present invention.

Preferably siRNA (see e.g. Nature Reviews “RNAi collection” (2003), Y. Dorsett, T. Tuschl, Nature Reviews Drug Discovery (2004) 3:318-329), antigens and antibodies directed against said antigens are used for the manufacture of a medicament for the treatment of keratokonjunktivitis, which is caused by AdV 8 (subgenus D).

Another aspect of the present invention relates to the use of an antigen according to the present invention for the manufacture of an antibody directed towards these antigens.

The antigens may also be used to produce antibodies against said antigens. In order to produce polyclonal antibodies the antigens are administered to an animal (e.g. mouse, goat, sheep, horse) and the antibodies formed are isolated. Monoclonal antibodies may also be produced by methods known in the state of the art (e.g. Kohler G, Milstein C., Nature 1975, 256:495-497).

Another aspect of the present invention relates to the use of an antibody directed towards an inventive antigen for the manufacture of a medicament for the passive immunisation of an individual against adenovirus infections.

Another aspect of the present invention relates to the use of an antibody directed against an antigen according to the present invention for the detection of an adenovirus in a sample.

These antibodies may be employed in many immunological assays such as e.g. enzyme linked immunosorbent assays (ELISA), radio immuno assays (RIA), immuno staining.

Another aspect of the present invention relates to a method for the detection of a single adenovirus serotype or an adenovirus subgenera or an adenovirus in a sample comprising the steps:

a) providing a sample containing a nucleic acid,

b) contacting said sample with at least one forward primer and at least one reverse primer having at least 85% identity with a portion of a nucleotide sequence of a hexon gene derived from one of the sequences SEQ ID No. 31 to 65,

c) subjecting the sample contacted with said primers to a nucleic acid amplification technique, and

d) optionally determining the presence of adenoviruses in said sample by detecting a nucleic acid amplification product.

This method allows determining specifically an adenovirus serotype in a sample, also if a pool of different adenovirus serotypes is present in said sample. In such a case it is advantageous to select suitable primers in regions of the hexon genes identified by SEQ ID No. 31 to 65 and being assigned to adenovirus serotype 6 (subgenus C), 8 (D), 9 (D), 10 (D), 13 (D), 14 (B), 15 (D), 18 (A), 19 (D), 20 (D), 22 (D), 23 (D), 24 (D), 25 (D), 26 (D), 27 (D), 28 (D), 29 (D), 30 (D), 31 (A), 32 (D), 33 (D), 36 (D), 37 (D), 38 (D), 39 (D), 42 (D), 43 (D), 44 (D), 45 (D), 46 (D), 47 (D), 49 (D), 50 (B) and 51 (D), respectively, which are not homologous to each other. This means that at least one of the primers chosen should be specific for a hexon gene of a distinct adenovirus serotype. In order to identify such sequences and sequence regions an alignment of the above mentioned sequences (optionally with sequences publicly available in databases such as the NCBI or the EMBL database) is a suitable instrument. Preferred variable nucleic acid sequence regions are found within the regions of the consensus sequences of SEQ ID No. 1 to 3 and 10 to 12, particularly within nucleic acid 390 and 1100, 1300 and 1480 of the respective nucleic acid sequences.

In order to identify distinct subgenera of AdV, alignments of the hexon genes of the members of a subgenus may be used to identify appropriate primers. For instance, SEQ ID No. 4 to 6 and SEQ ID No. 7 and 8 may be used to construct primers for the identification of AdVs of subegenera A and C, respectively. In particular primers hybridizing at the 5′-end portions of SEQ ID No. 4 and 7 and/or at the 3′-end portions of SEQ ID No. 6 and 8 comprising 1 to 400, 50 to 350 nucleic acids may preferably be employed.

Preferably the nucleic acid amplification technique is performed with at least one oligonucleotide probe hybridising in between of the at least one forward primer and the at least one reverse primer within the hexon gene of an Adenovirus and being derived from one of the sequences SEQ ID No. 31 to 65.

According to another preferred embodiment of the present invention the at least one oligonucleotide probe is tagged with a dye, preferably a fluorescent dye, and optionally with a quencher.

### BRIEF DESCRIPTION OF THE DRAWINGS

The present invention is further illustrated by the following figures and example, without being restricted there to.

FIG. 1 shows amplification plots (A) and standard curves (B).

Serial dilution of adenoviral DNA (species A, serotype 2) ranging from 1×10² to 1×10⁷ virus particles per reaction. The analysis was done in duplicates using the ACF RQ-PCR system.

FIG. 2 shows amplification plots (A) and standard curves (B) and

FIG. 3 contains sequences disclosed herein.

### DESCRIPTION OF ILLUSTRATIVE EMBODIMENTS

Serial dilution of adenoviral DNA (species B, serotype 7) ranging from 1×10² to 1×10⁷ virus particles per reaction. The analysis was done in duplicates using the BDE system.

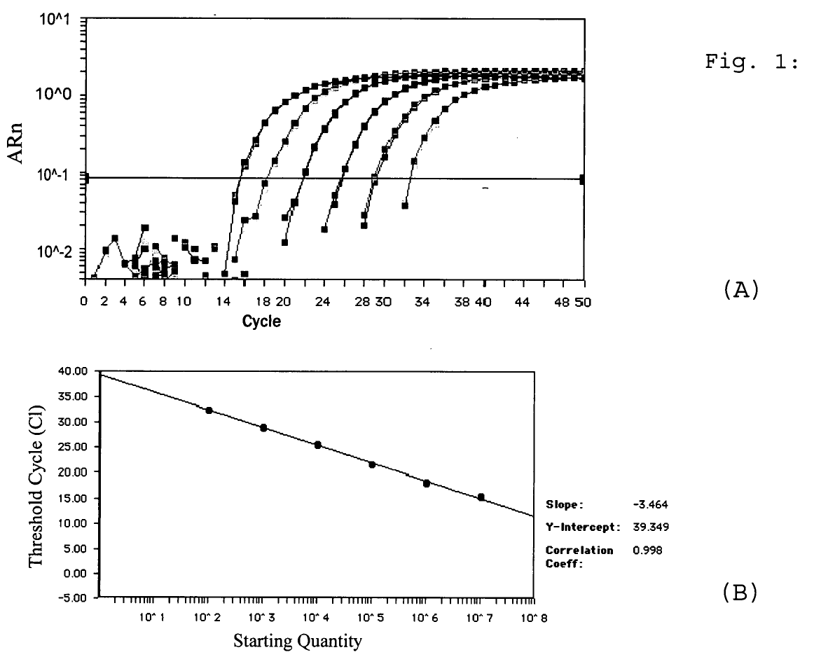

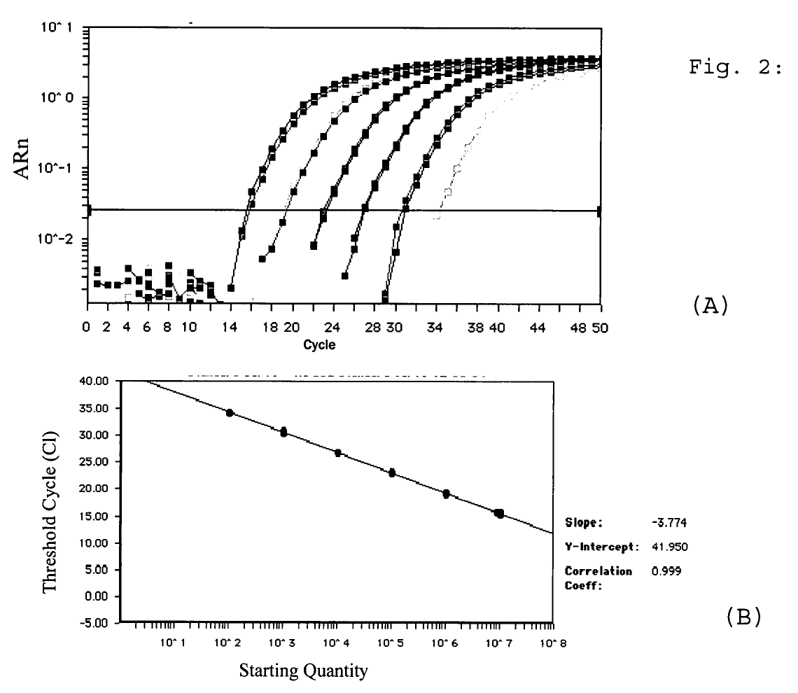

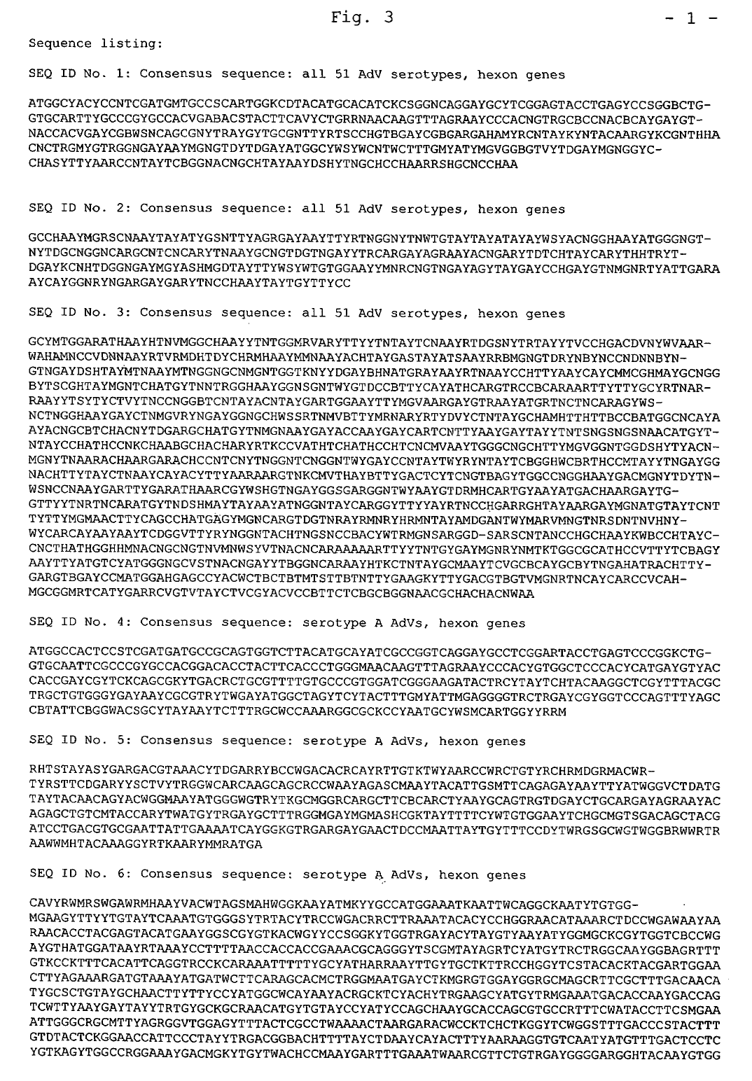

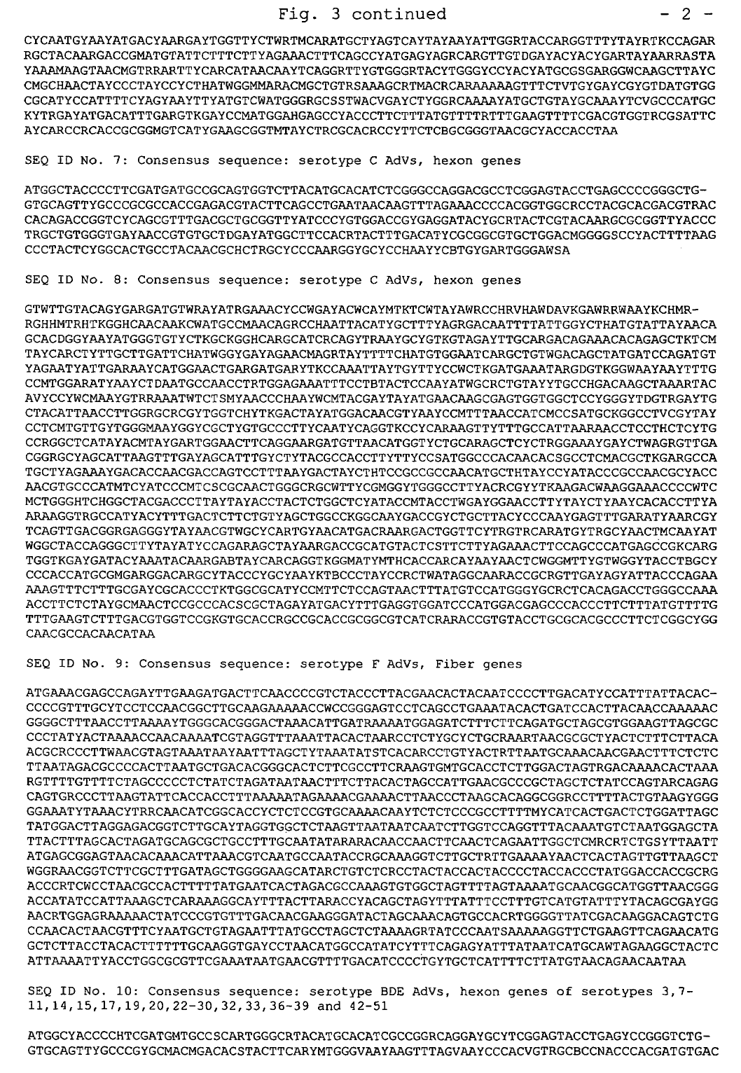

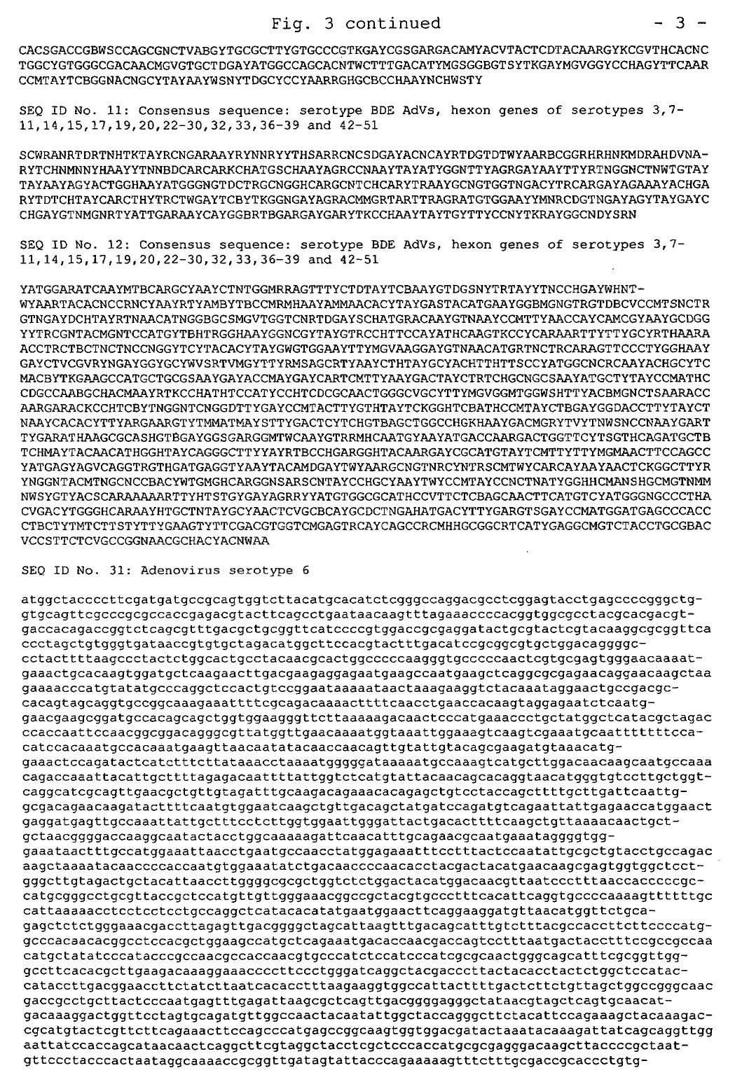

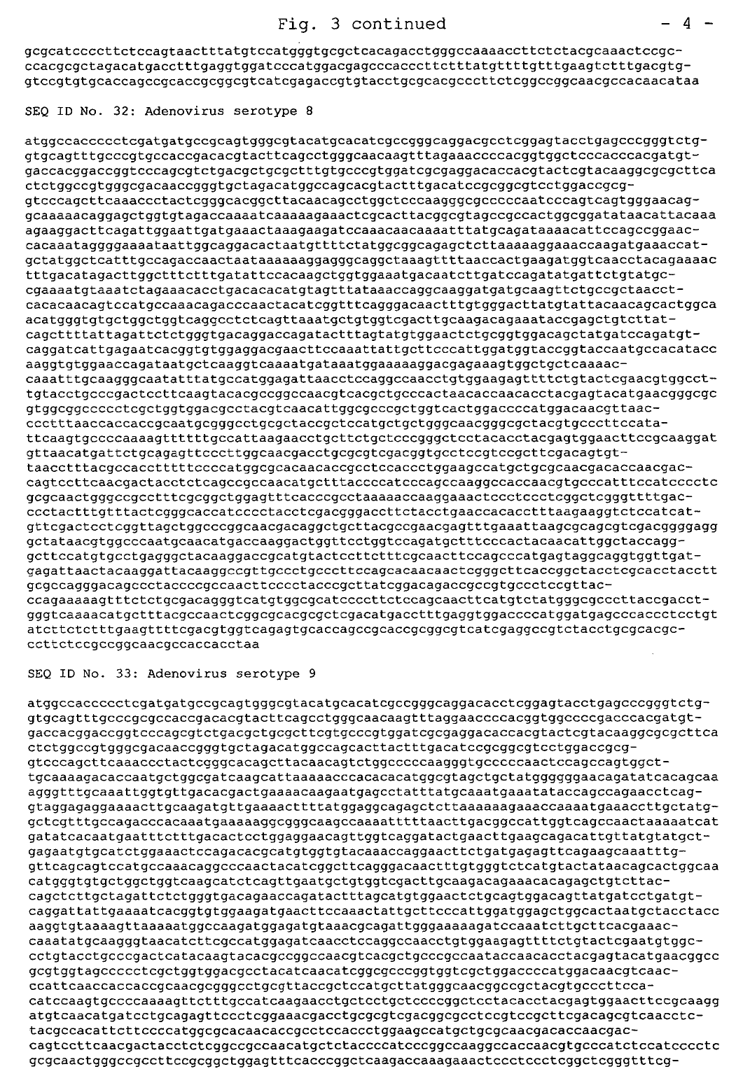

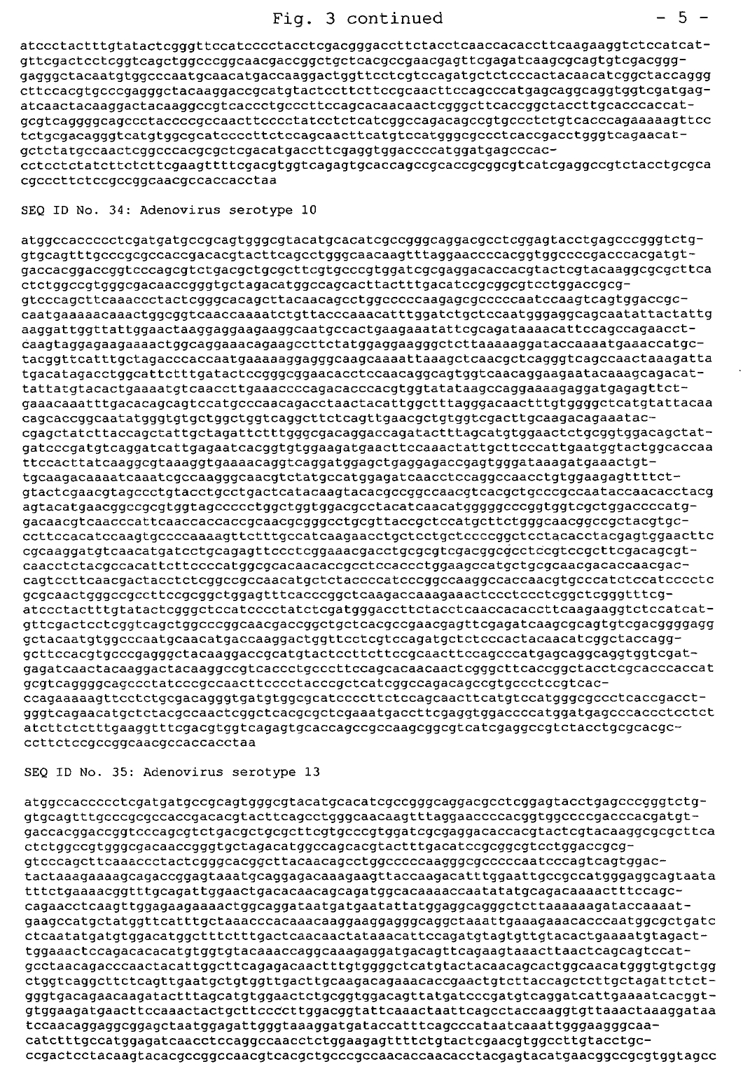

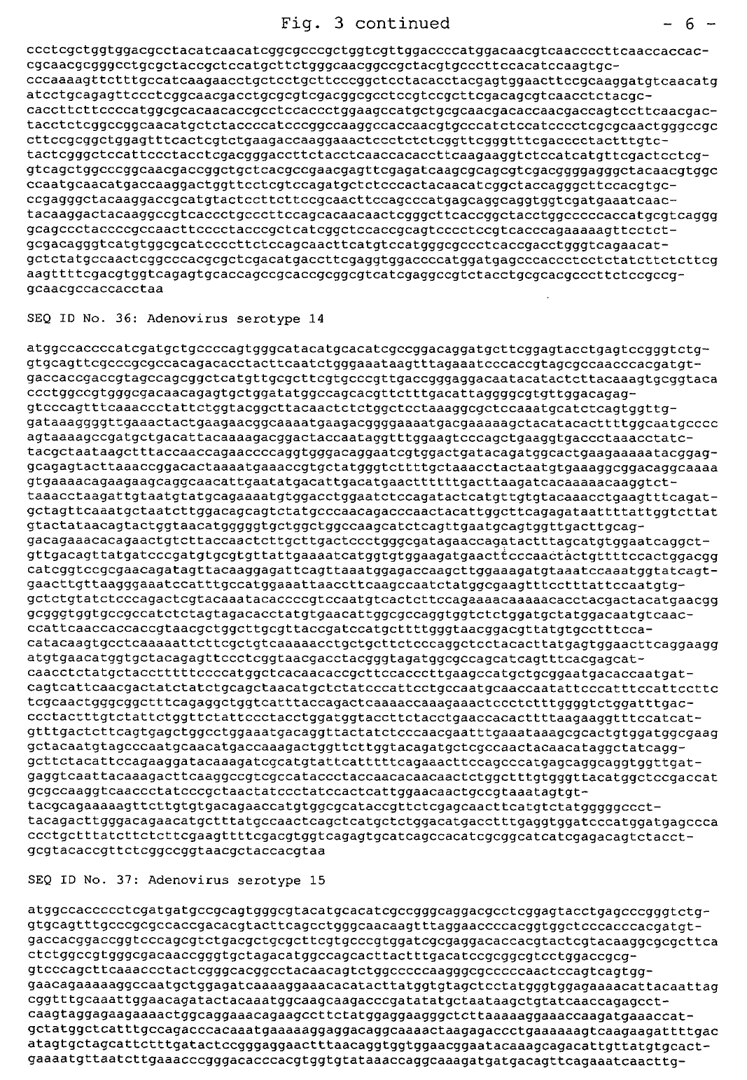

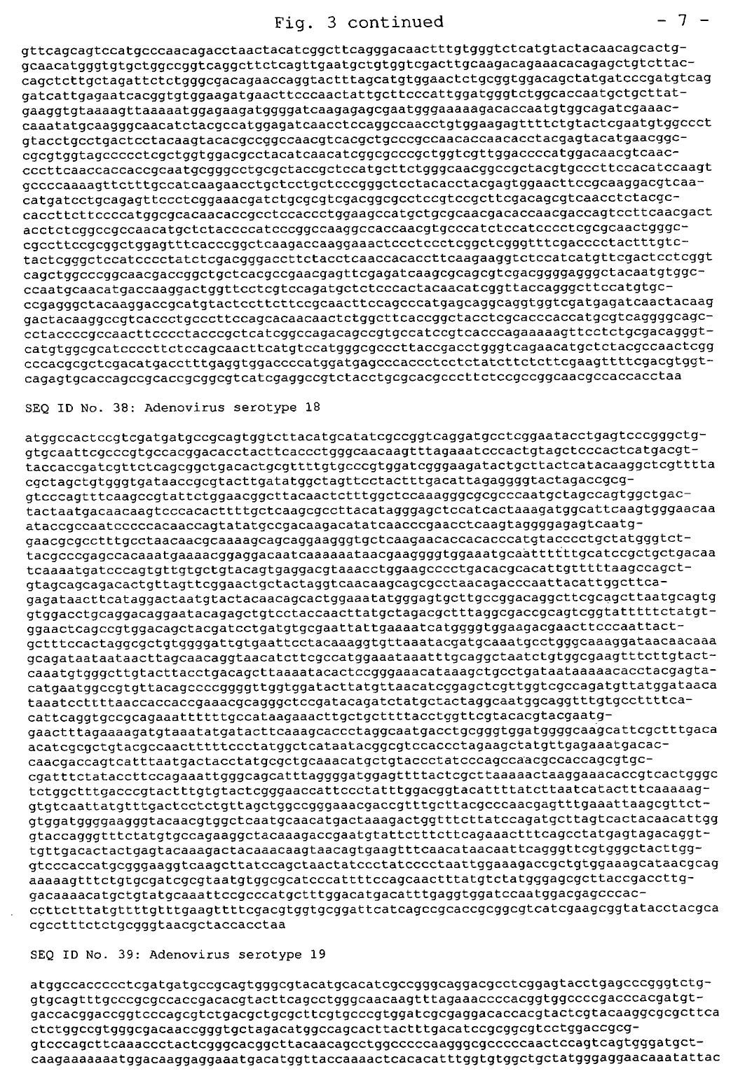

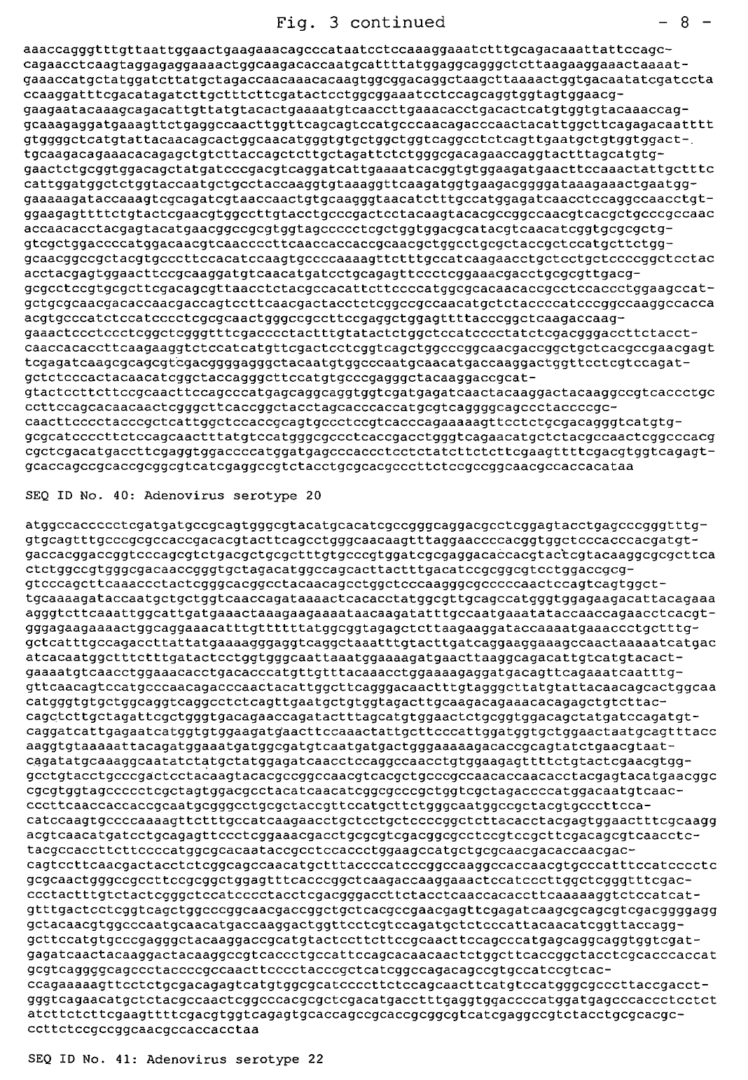

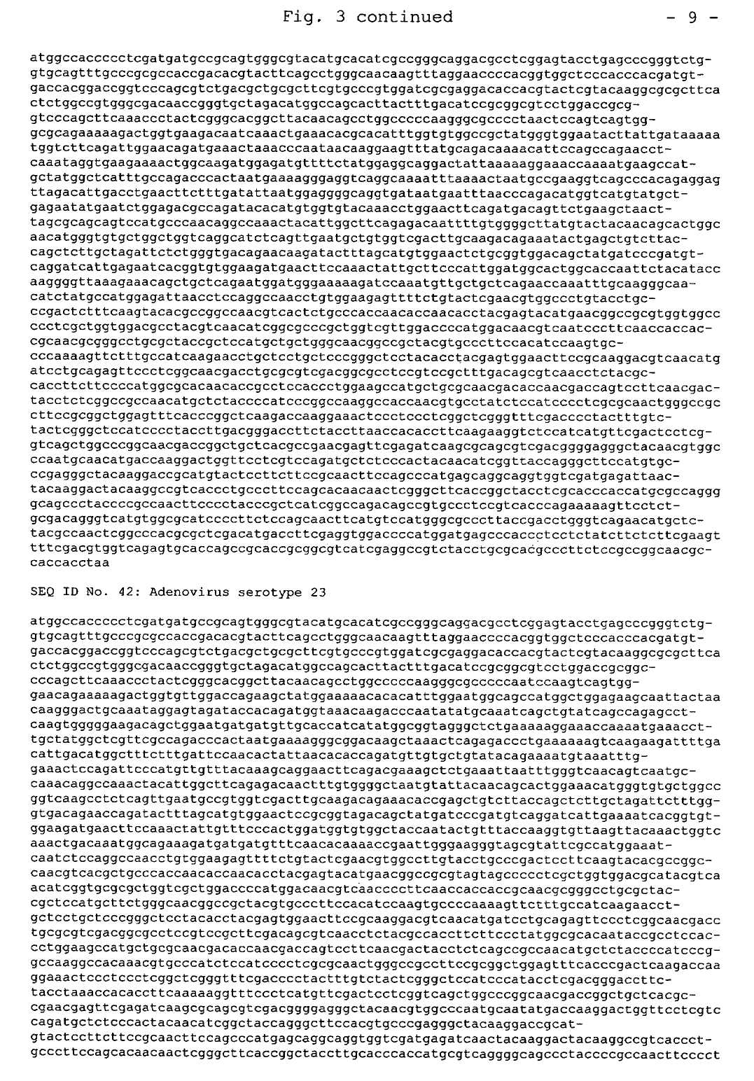

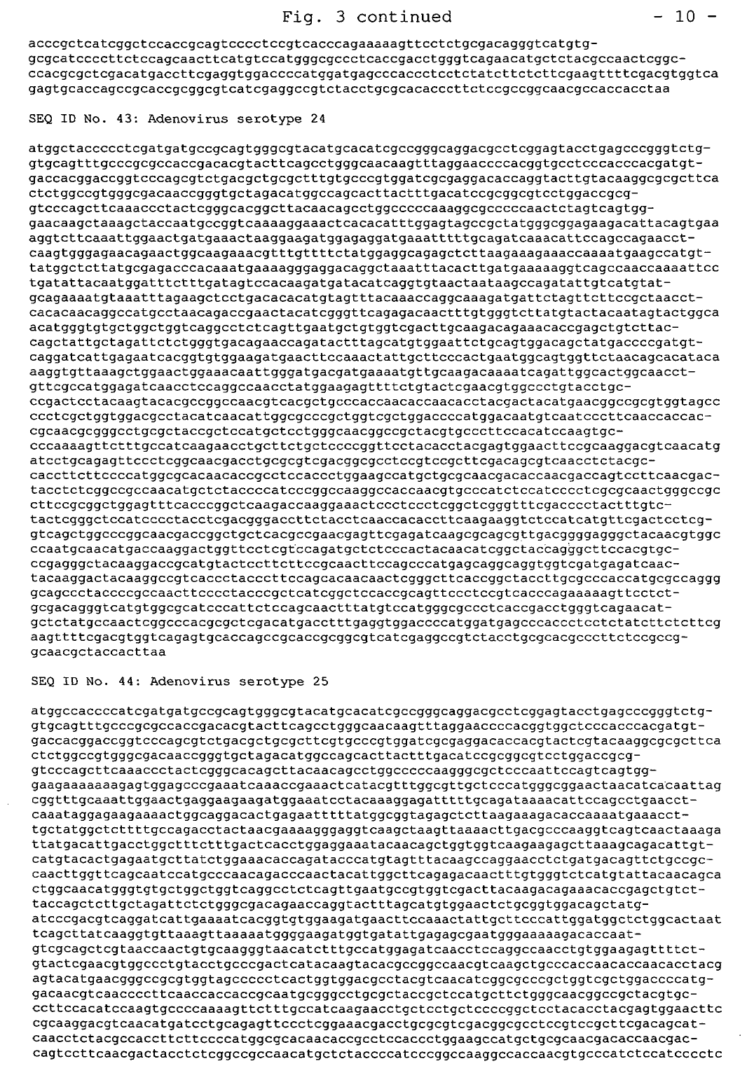

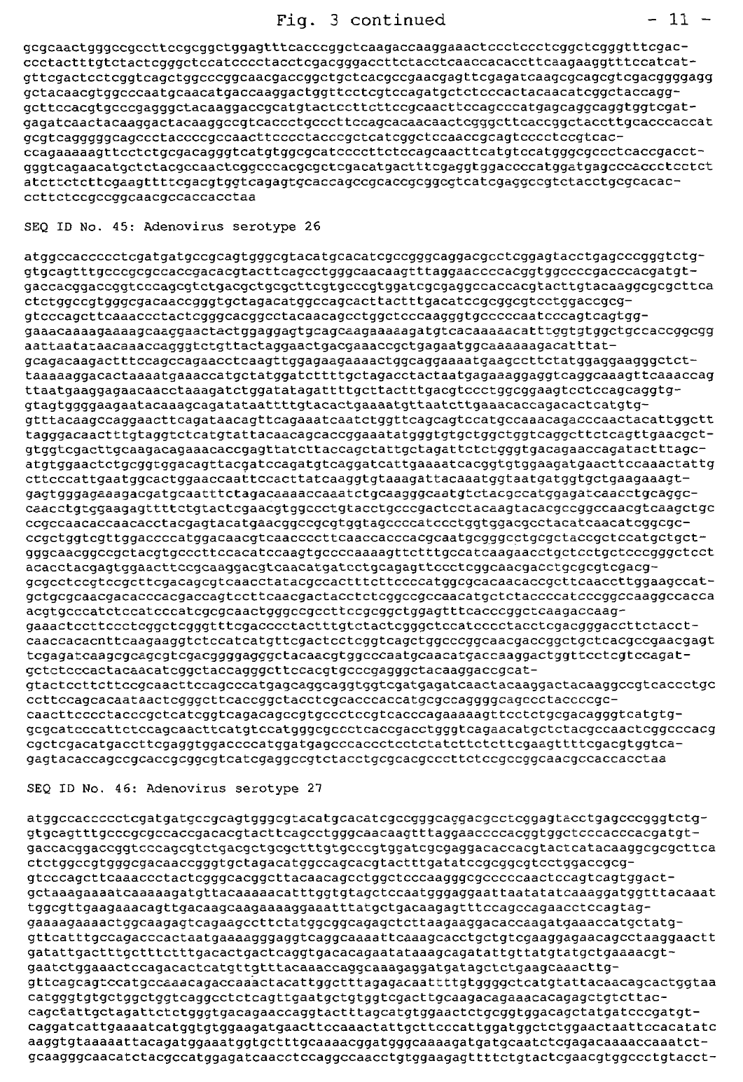

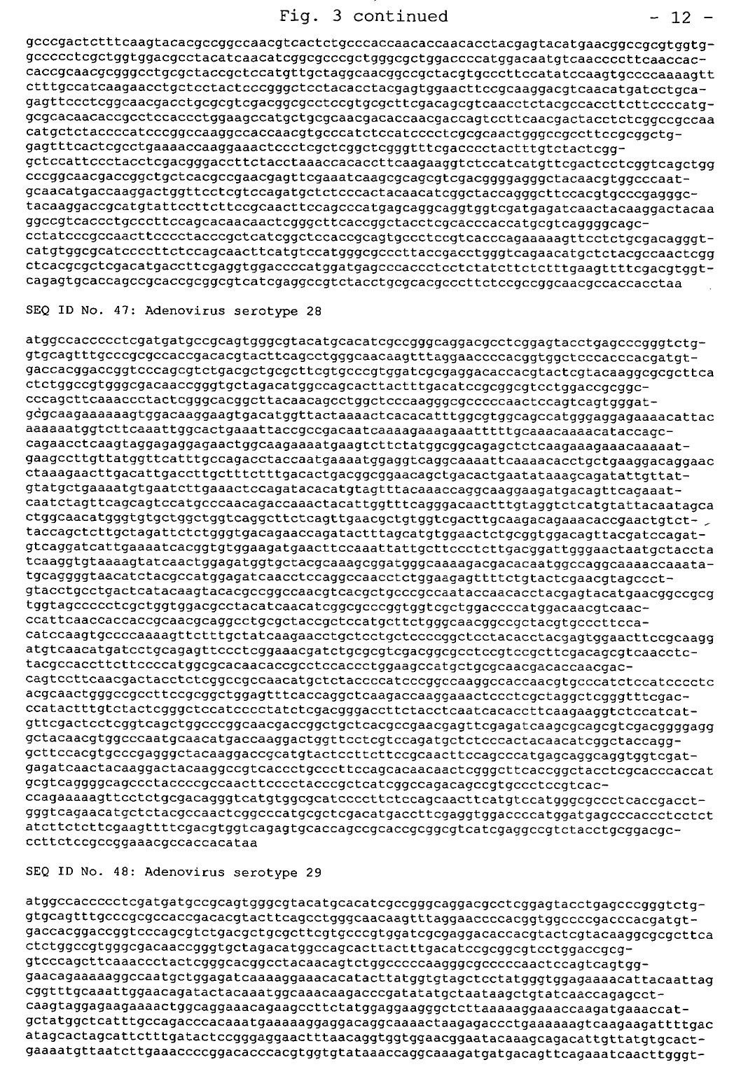

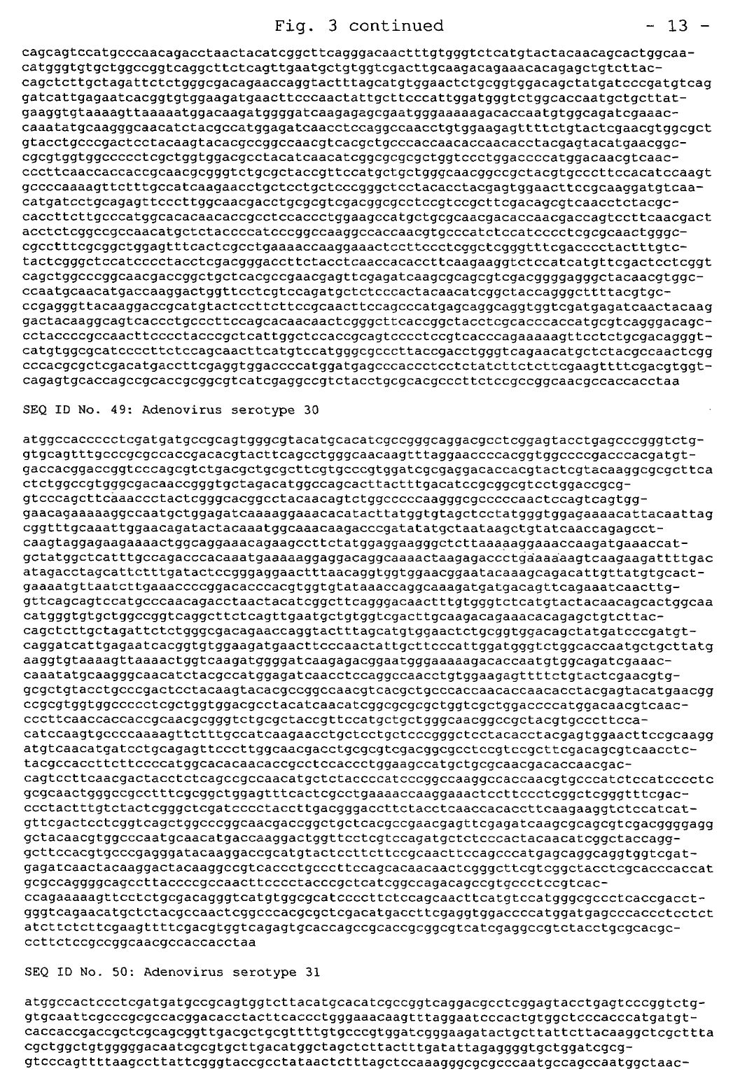

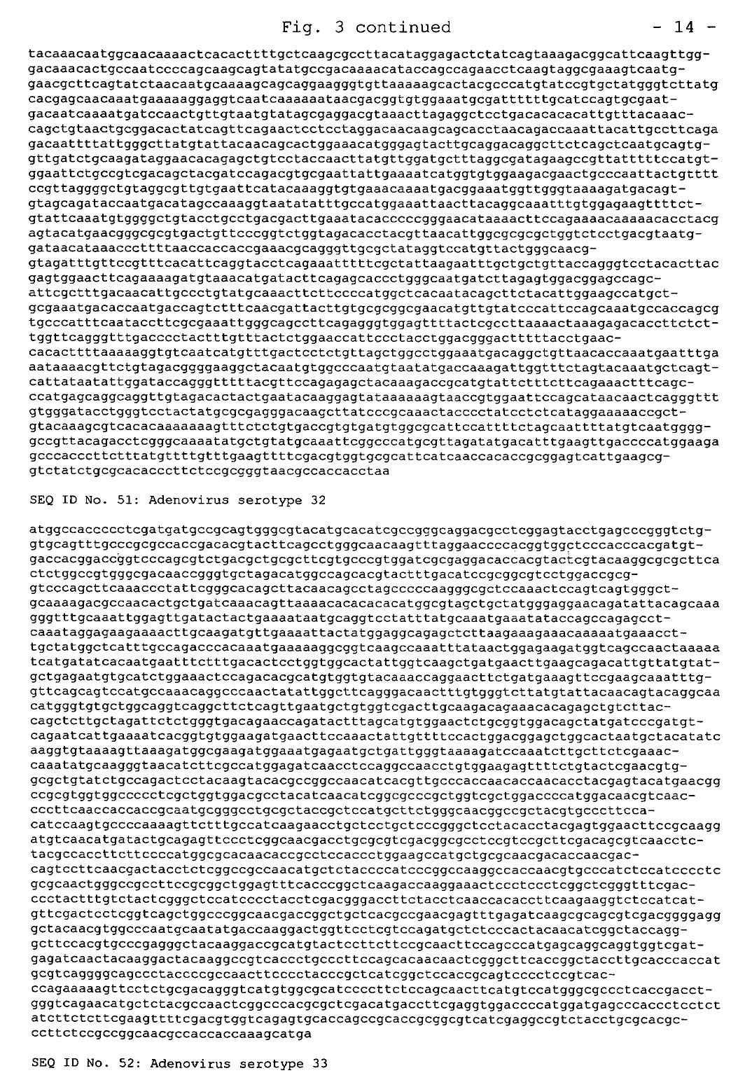

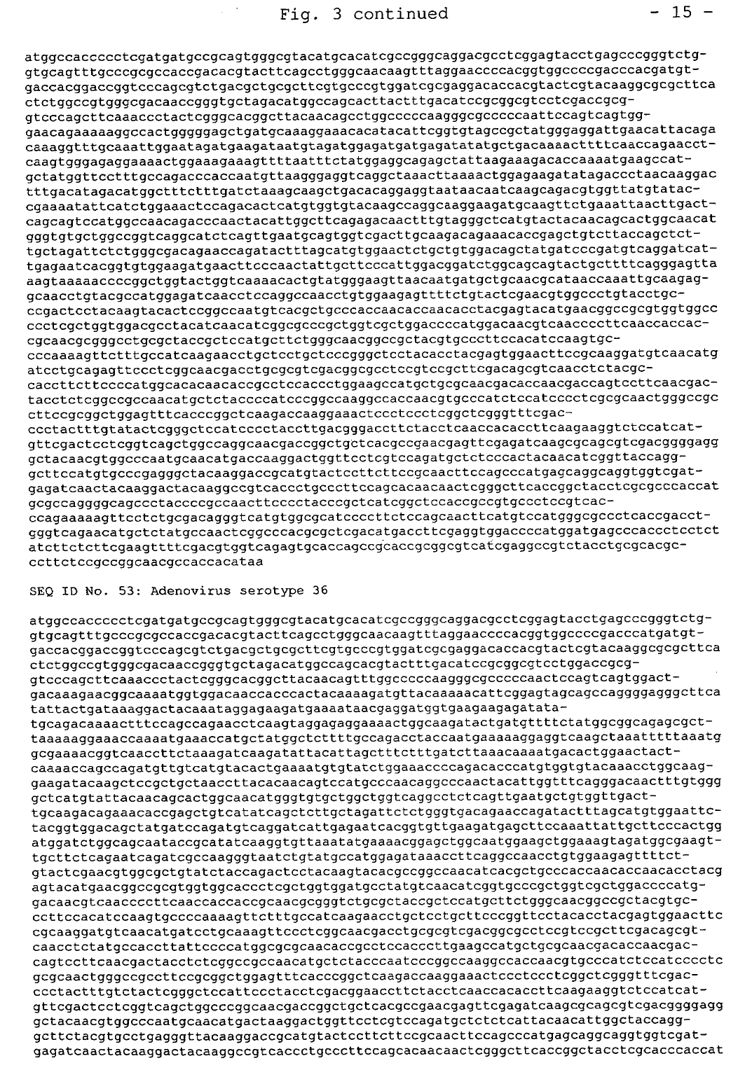

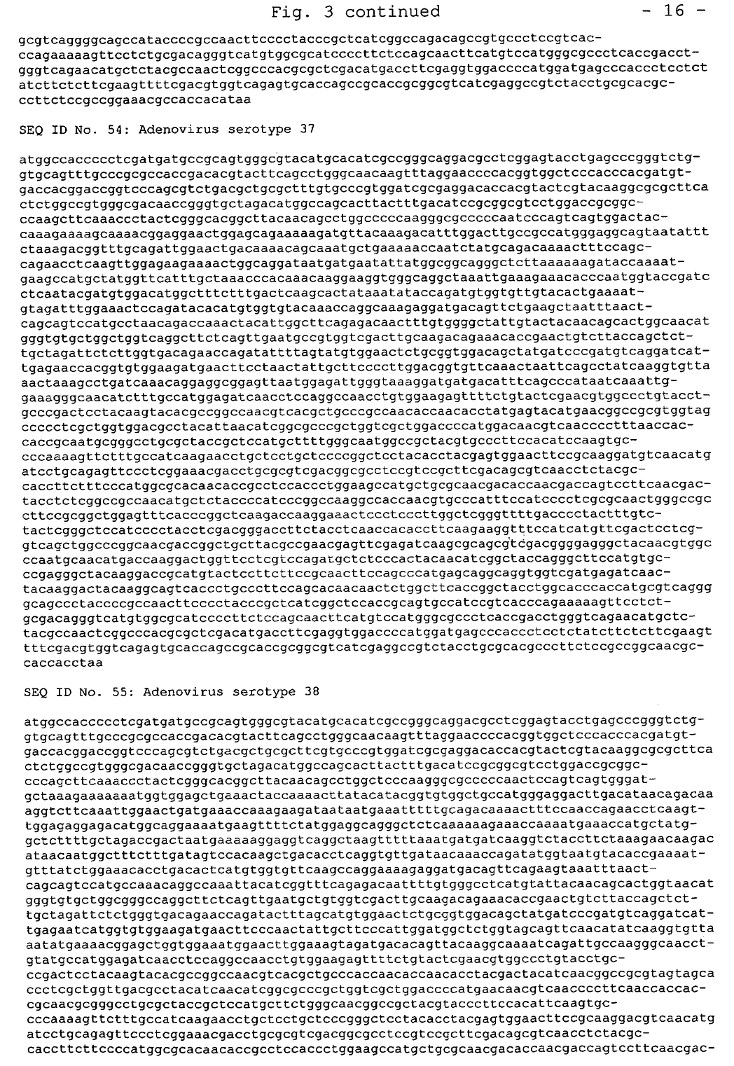

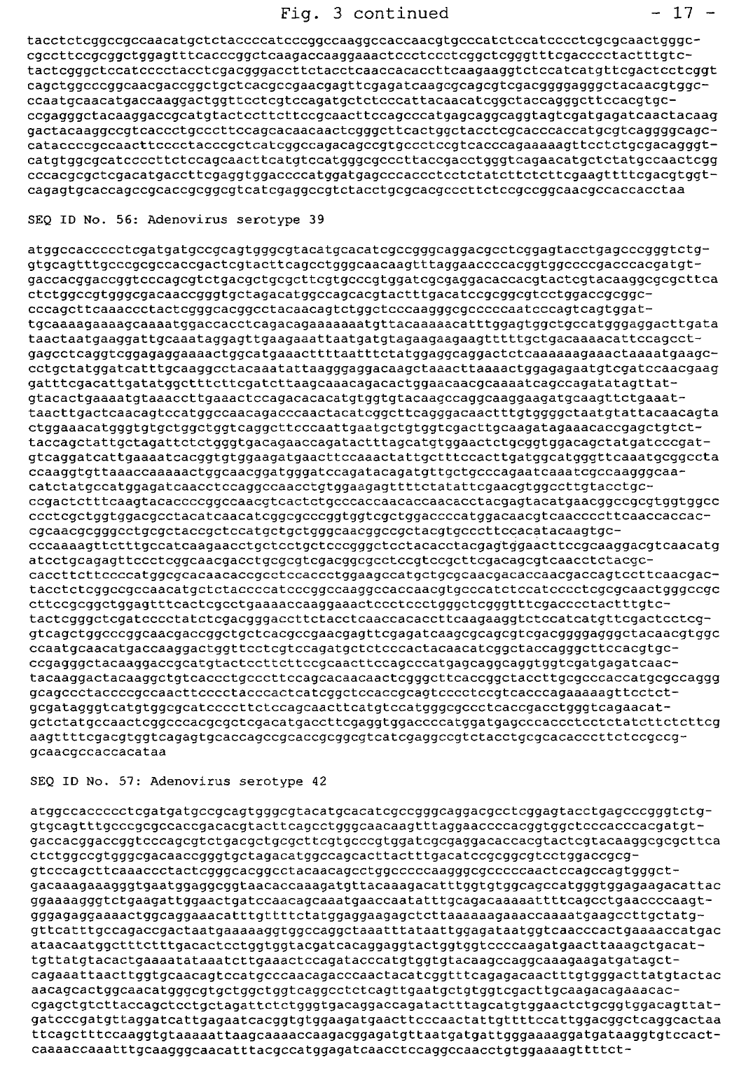

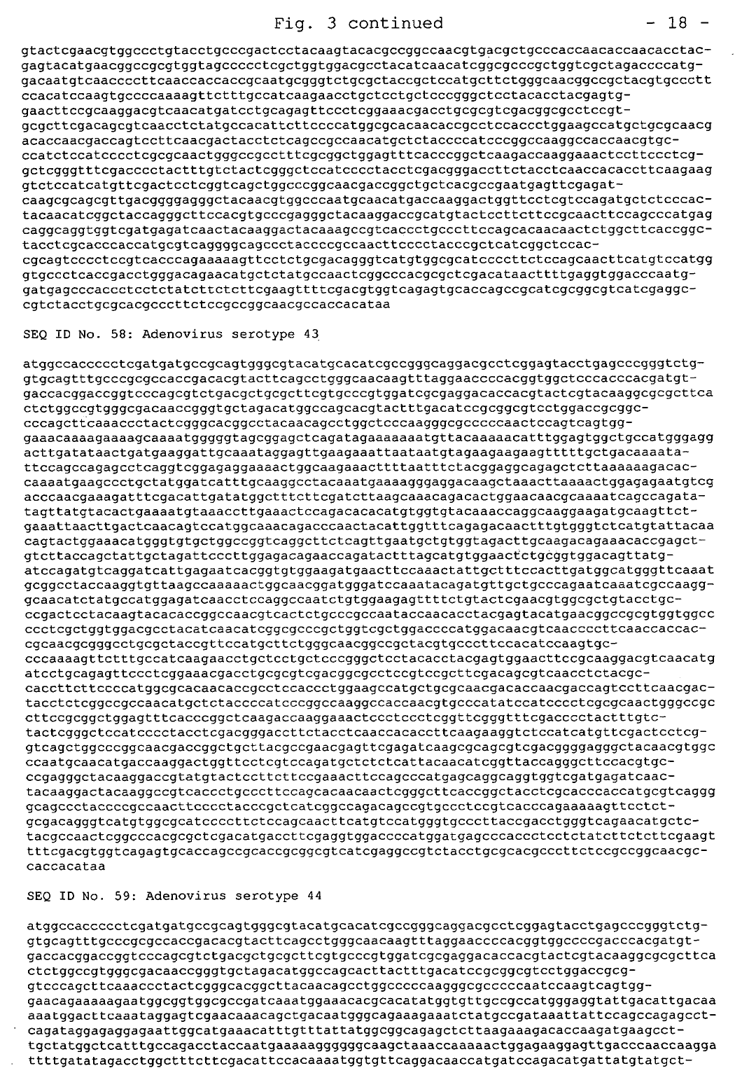

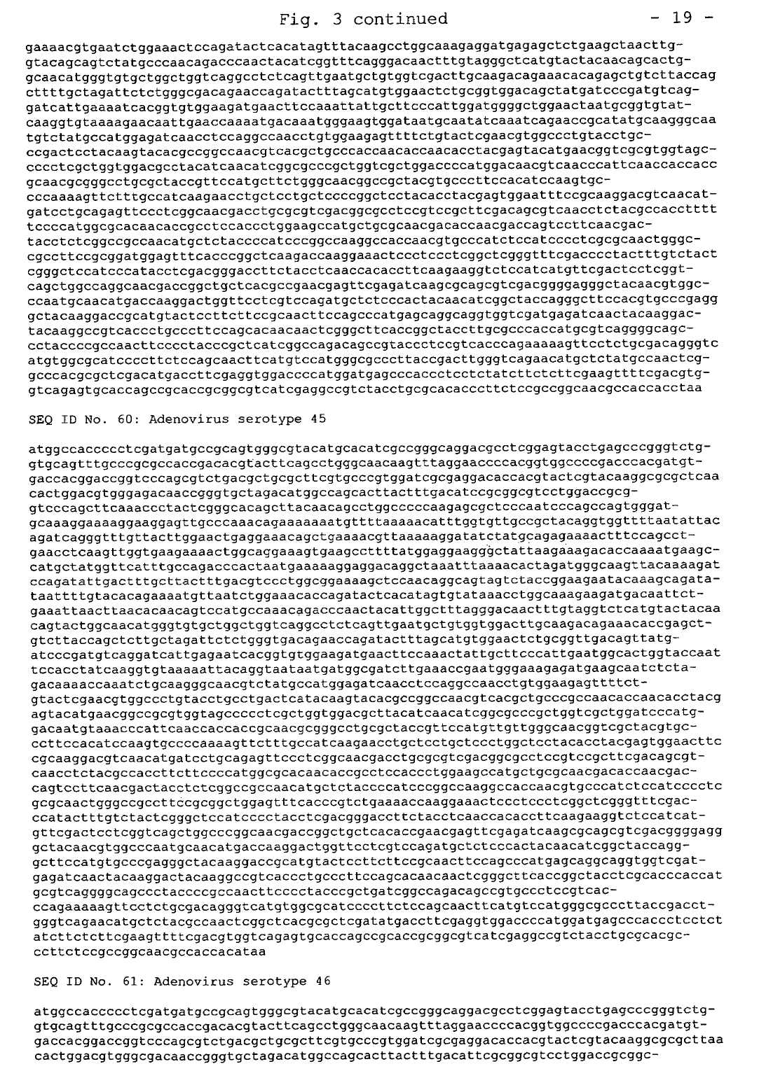

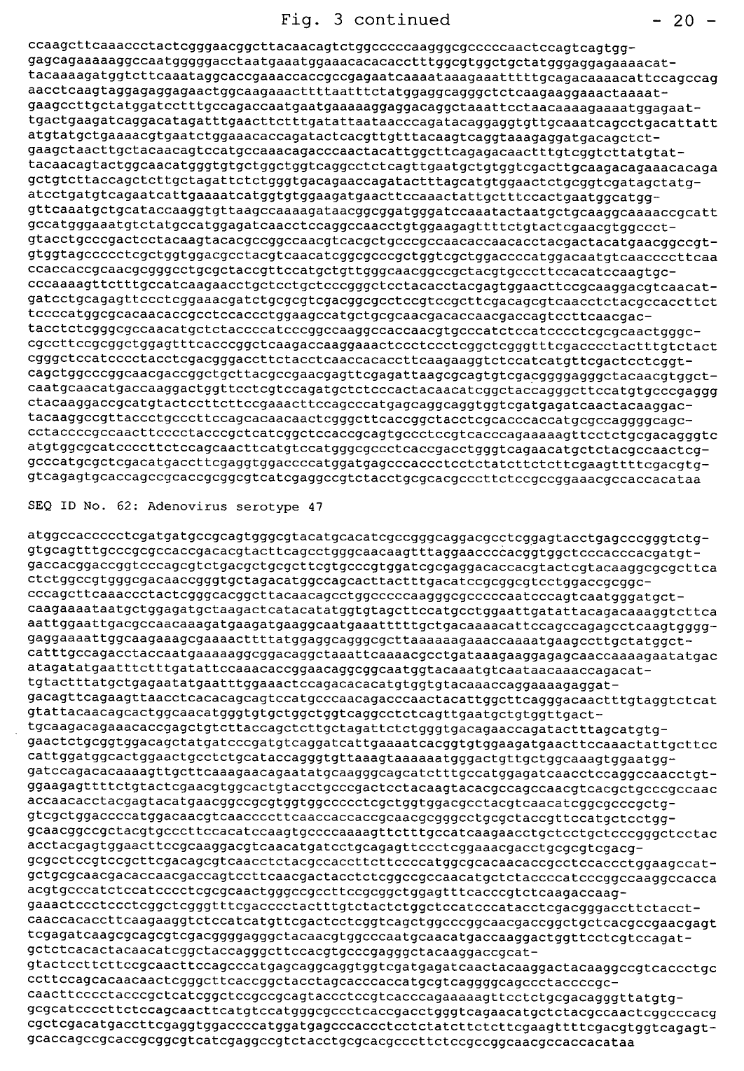

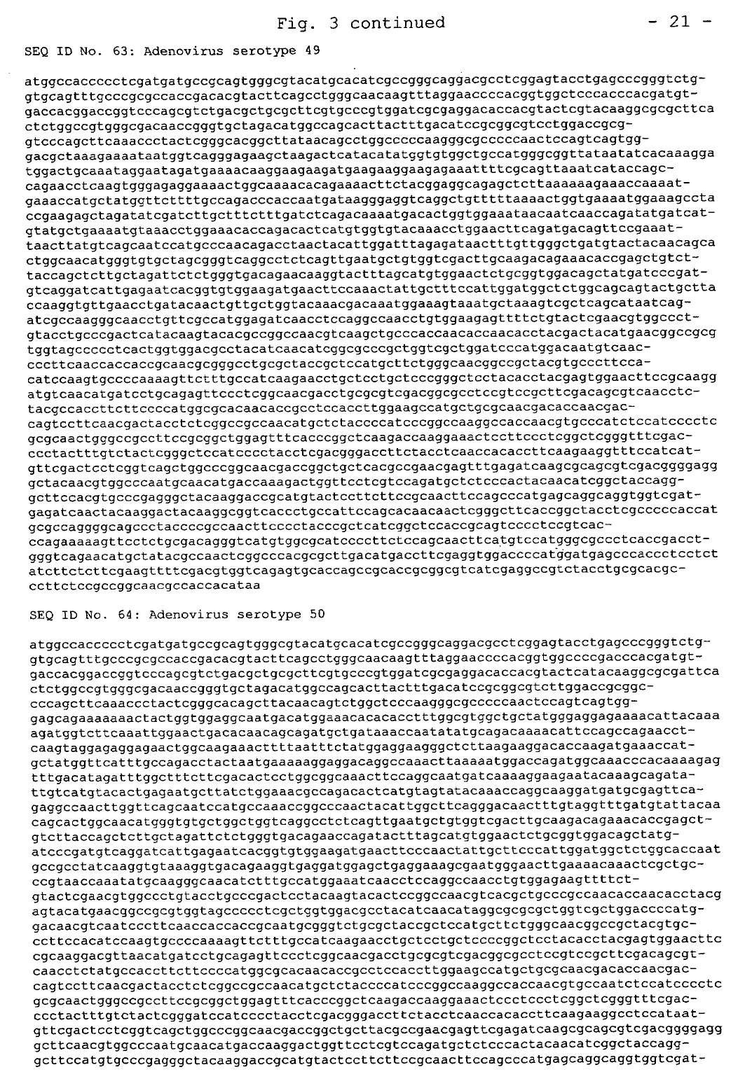

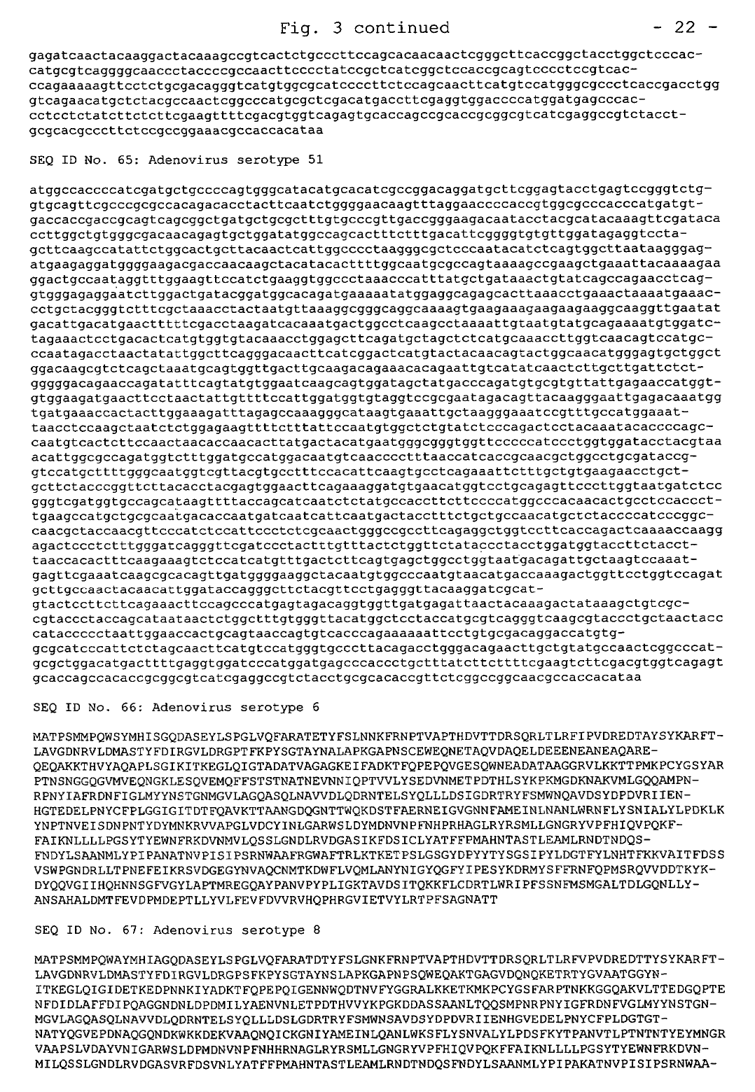

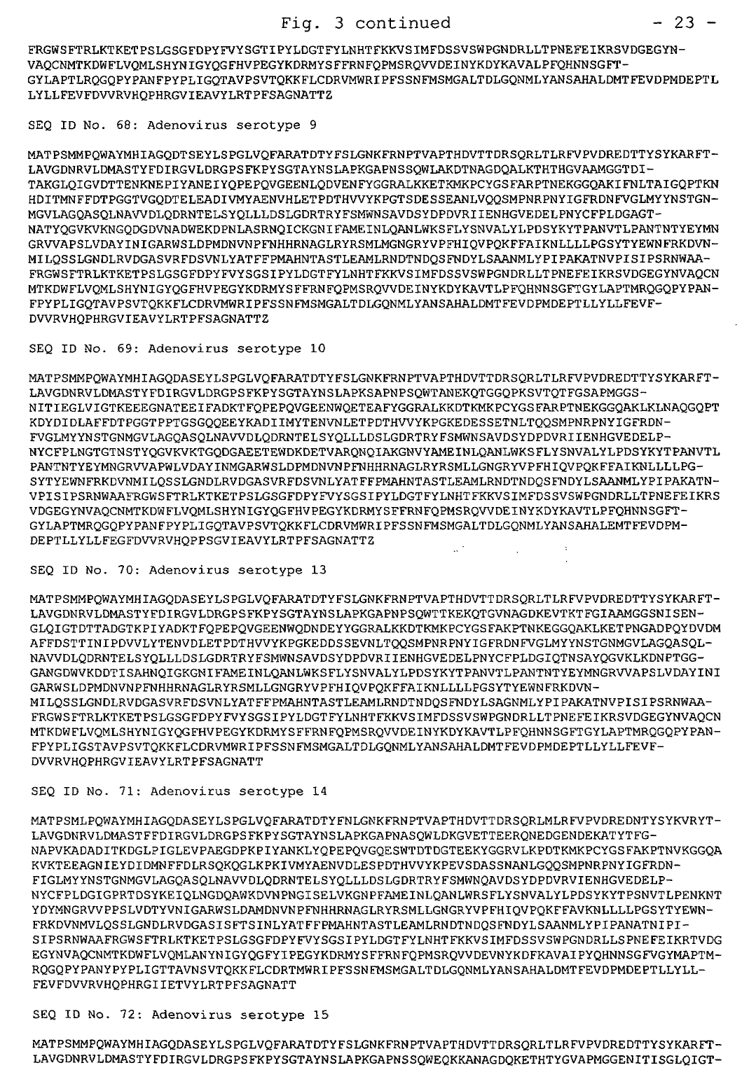

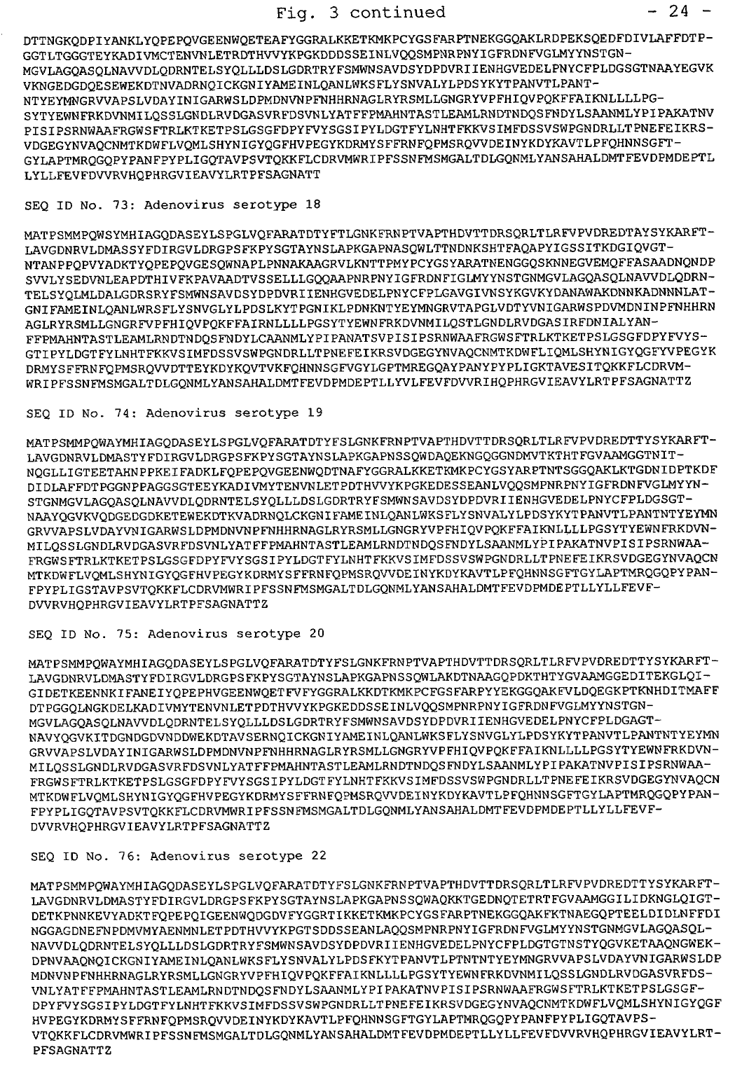

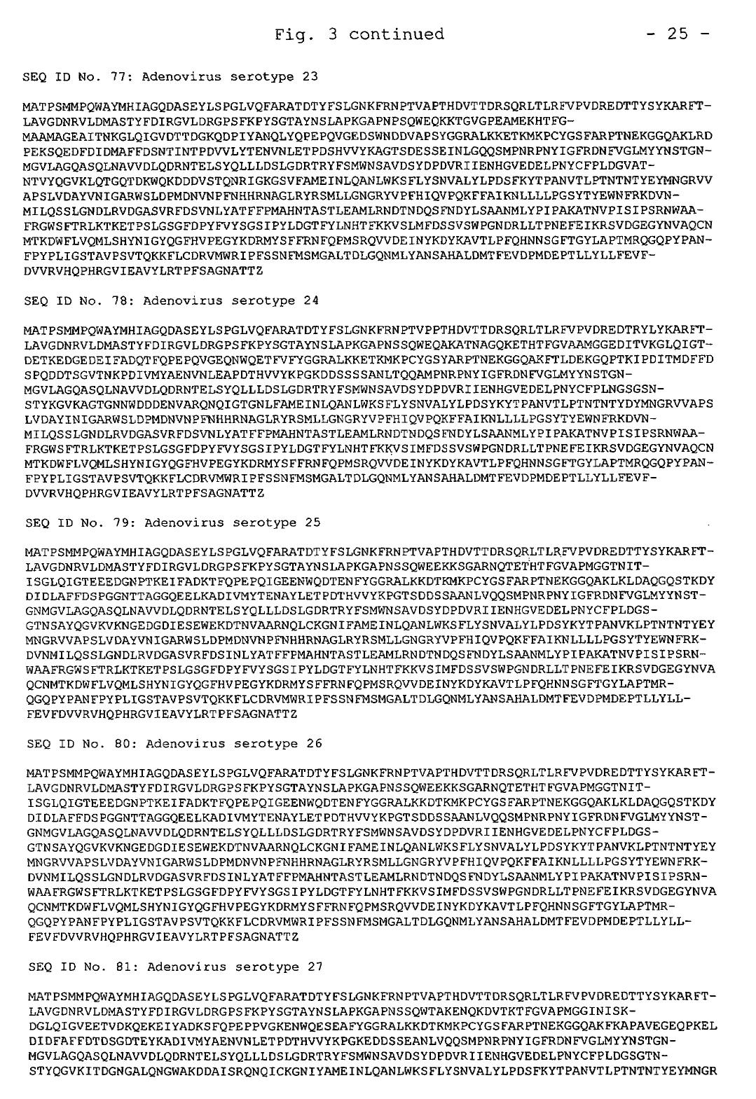

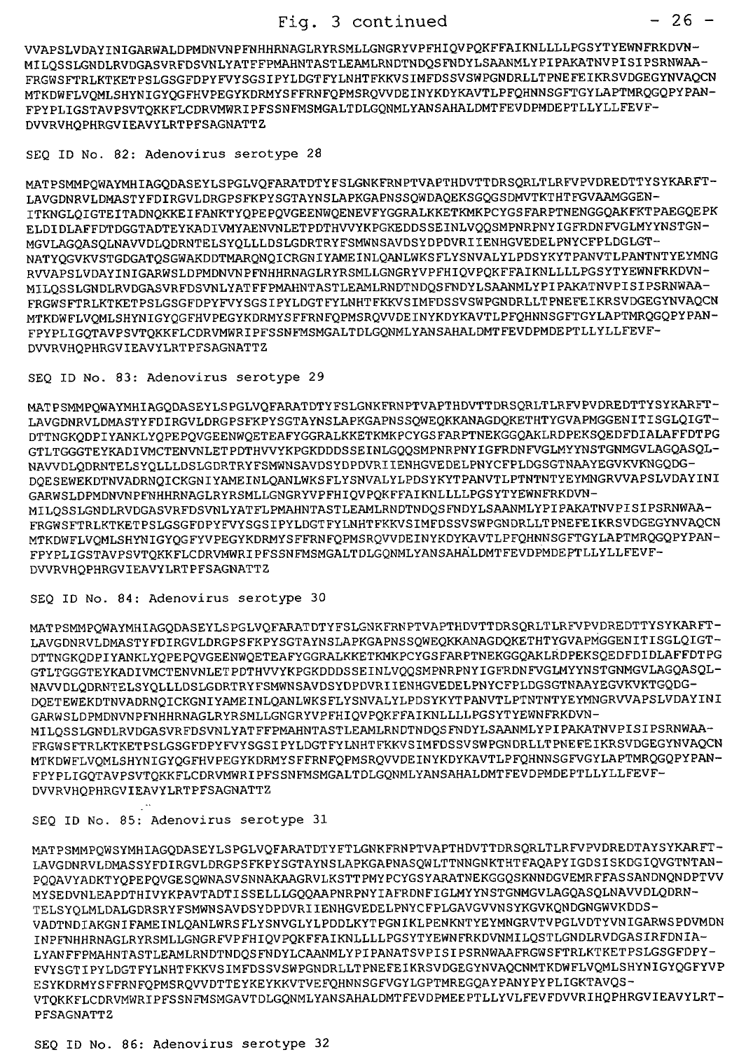

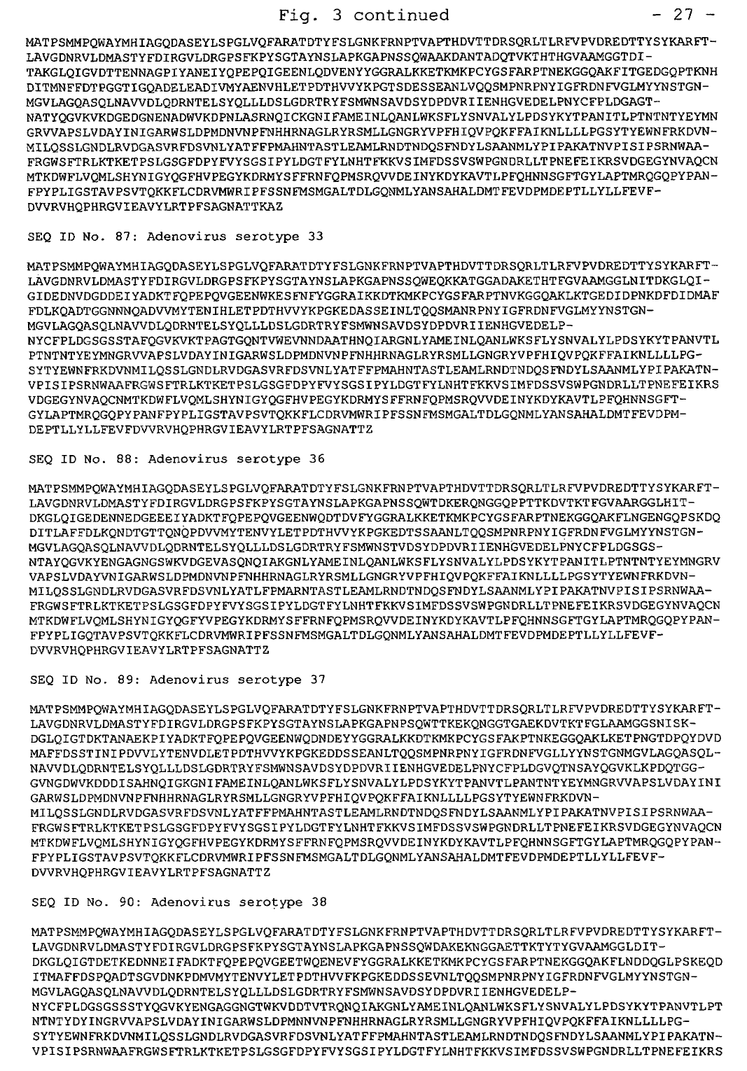

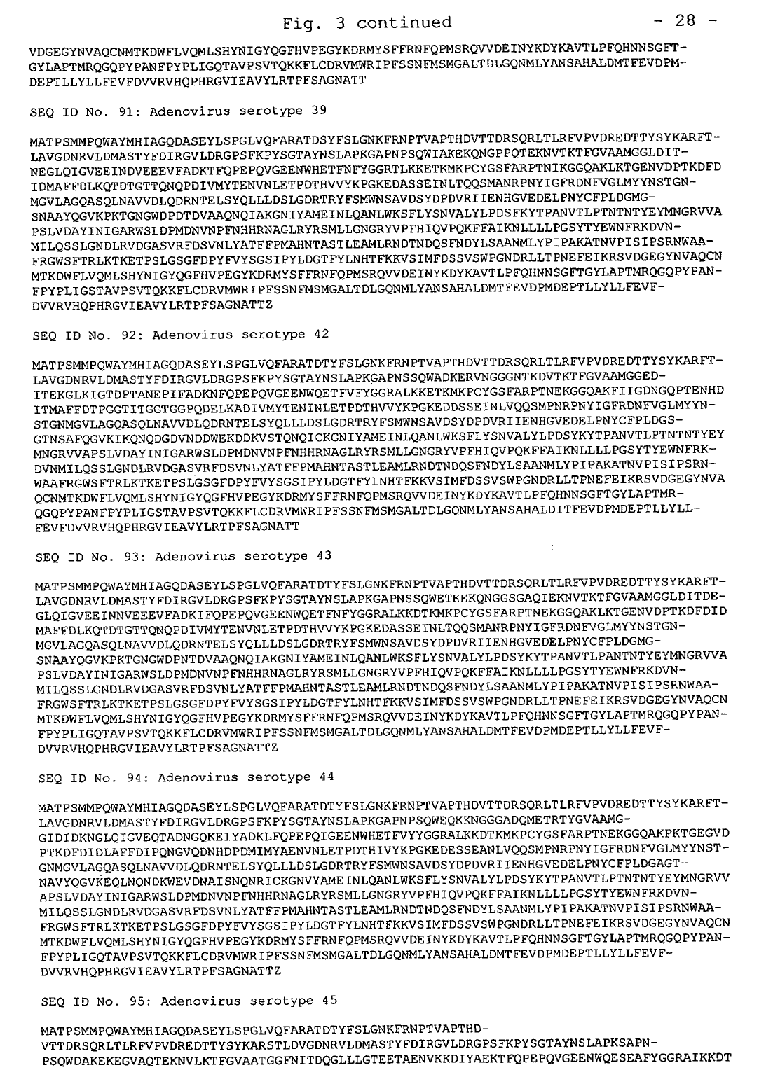

The amplification plots displayed in FIG. 1 and FIG. 2 are concordant with those established using serial dilutions of serotypes representative of other AdV species. Therefore, the amplification plots generated on the basis of the above serial dilutions may serve as external standard curves for the ACF and BDE RQ-PCR detection systems, respectively.

### EXAMPLE

### Materials and Methods:

Clinical samples

Forty five consecutive pediatric patients undergoing allogeneic stem cell transplantation at St. Anna Children's Hospital, Vienna, Austria, between May, 2003, and September, 2004, were screened for the presence of latent AdV infection in peripheral blood (PB) leukocytes prior to transplantation by the two-reaction PCR technique presented. Subsequently, prospective virus screening in PB and stool was performed at short intervals until immunologic reconstitution, as described (21). In AdV positive samples, virus load was determined by real-time PCR as outlined below. In total, more than 1.000 clinical samples were screened by the technique described.

Isolation of DNA

Cell culture supernatants derived from reference strains of all 51 serotypes (provided by the Department of Virology, University of Rotterdam, The Netherlands and A. Heim, Department of Virology, Medical University Hannover, Germany) were lysed and DNA was extracted using the QIAGEN DNA Mini Kit (QIAGEN, Hilden, Germany). DNA from peripheral blood was extracted using the same protocol. For isolation of DNA from stool samples, the QIAmp DNA Stool Mini Kit was used (QIAGEN, Hilden, Germany) according to the manufacturer's recommendations.

Real-Time Quantitative PCR

Primers and probes for the two-reaction pan-adenovirus detection system were placed within conserved regions of the hexon and the fiber genes, according to sequences available in part from a public database (National Center for Biotechnology Information—NCBI) and, for the most part, on the basis of new hexon gene sequence data provided in the present patent application: the primer and probe design was based on the alignment of previously published hexon and fiber gene sequences of the serotypes 1, 2, 3, 4, 5, 7, 11, 12, 16, 17, 21, 34, 35, 40, 41 and 48, and on the sequence data of the remaining 35 AdV serotypes determined in the laboratory.

Prior to experimental testing, the primer and probe sequences were controlled by standard nucleotide-nucleotide BLAST (NCBI, www.ncbi.nml.nih.gov/BLAST/) for the absence of homology with any other relevant organism. All primers and probes included in the two-reaction assay (see Table 1) were designed with the help of the Primer Express software (Applied Biosystems, Foster City, Calif.) to work under identical cycling conditions.

TABLE 1

| | | Nucleotide positions | |
|---|---|---|---|
| | | (Hexon gene H or | Conc. |
| Name | Sequences | Fiber gene) | (μM) |
| Ad ACF for I | ggk ctg gtg caa ttc gcc | 79-96 (H) | 0, 3 |
| | (SEQ ID No. 13) | (of SEQ ID No. 4) | |
| Ad ACF rev I | cac ggg cac aaa acg ca | 197-213 (H)* | 0, 3 |
| | (SEQ ID No. 17) | (of SEQ ID No. 4) | |
| Ad ACF for II | acc tgg gcc aaa acc ttc tc | 1706-1725 (H) | 0, 3 |
| | (SEQ ID No. 14) | (of SEQ ID No. 8) | |
| Ad ACF rev II | cgt cca tgg gat cca cct c | 1762-1780 (H)* | 0, 9 |
| | (SEQ ID No. 18) | (of SEQ ID No. 8) | |
| Ad ACF for III | ccc gtg ttt gac aac gaa gg | 1308-1328 (F) | 0, 3 |
| | (SEQ ID No. 22) | (of SEQ ID No. 9) | |
| | | 31277-31282 | |
| | | of Acc.No. L19443 | |
| Ad ACF rev III | tta gag cta ggc ata aat | 1395-1421 (F)* | 0, 3 |
| | tct aca gca | (of SEQ ID No. 9) | |
| | (SEQ ID No. 23) | 31363-31389 | |
| | | of Acc.No. L19443 | |
| Ad ACF probe I | cca cgg aca cct act tca | 101-125 (H) | 0, 2 |
| | ccc tgg g | (of SEQ ID No. 4) | |
| | (SEQ ID No. 24) | | |
| Ad ACF probe II | aac tcc gcc cac gcg cta ga | 1732-1751 (H) | 0, 2 |
| | (SEQ ID No. 25) | (of SEQ ID No. 9) | |
| Ad ACF probe III | atc gac aag gac agt ctg | 1357-1387 (F) | 0, 2 |
| | cca aca cta acg | (of SEQ ID No. 9) | |
| | (SEQ ID No. 30) | 31326-31355 | |
| | | of Acc.No. L19443 | |
| Ad BDE for | aca tgc aca tcg ccg g | 35-50 (H) | 0, 4 |
| | (SEQ ID No. 15) | (of SEQ ID No. 10) | |
| Ad BDE rev | cgg tcs gtg gtc aca tc | 163-179 (H)* | 0, 4 |
| | (SEQ ID No. 19) | (of SEQ ID No. 10) | |
| Ad BDE probe I | cgg gtc tgg tgc agt | 77-91 (H) | 0, 2 |
| | (SEQ ID No. 26) | (of SEQ ID No. 10) | |
| Ad BDE probe II | cgg gtt tgg tgc agt | 77-91 (H) | 0, 2 |
| | (SEQ ID No. 27) | (of SEQ ID No. 10) | |

* complementary strand

PCR reactions were set up in a total volume of 25 μl, including 12.5 μl TaqMan Universal Master Mix (Eurogentec, Belgium) and 6 pl template DNA. The primer and FAM (6-carboxyfluorescein)-labelled probe concentrations are listed in Table 1. Amplifications were carried out using the ABI Prism 7700 or 7900 (Applied Biosystems, Foster City, Calif.) for a total of 50 cycles. After an initial denaturation step for 10 minutes at 95° C., each cycle consisted of denaturation for 15 seconds at 95° C. and annealing and primer extension for 60 seconds at 60° C. Strict precautions were undertaken to prevent contamination of PCR reactions with exogenous products as described (20). A dUTP glycosylase step was performed prior to each PCR reaction. Each DNA sample was analyzed in duplicate, and multiple negative controls were included in each assay. DNA isolates derived from different AdV serotypes served as positive controls. An additional control for efficient DNA extraction and the presence of inhibitors of amplification was the seal herpes virus PHHV (provided by the Department of Virology, University of Rotterdam, The Netherlands), which was spiked at defined concentrations into clinical samples prior to isolation of DNA. Successful extraction of viral DNA and absence of inhibitory effects was documented by real-time PCR quantification of PHHV copy number in each clinical sample. Samples that tested AdV positive were controlled by the species-specific RQ-PCR described earlier (21). For the quantification of virus load, external standard curves were established using serial dilutions of quantified virus preparations derived from reference strains of serotypes 2, 4, 7, 18, 24 and 40, as representatives of all six AdV species. For assessment of virus copies per cell, a single copy gene (beta2 microglobulin) was quantified in parallel by real-time PCR (26). When investigating largely cell-free liquids, such as plasma, quantitative results were expressed as the number of virus copies per ml. In stool samples, the virus copies were calculated per gram material, and intracellular virus copies were indicated per 10⁶ cells.

Results and Discussion

Specificity of the Assay

The analysis of DNA sequences of the hexon gene in the entire panel of currently known human adenoviruses revealed a certain level of similarity between serotypes belonging to the AdV species A/C/F and B/D/E, respectively. The degree of homology within the two groups of AdV species permitted the establishment of a single RQ-PCR reaction covering all serotypes of the species A, C and F (reaction-1) and a second reaction facilitating the detection of all serotypes of the species B, D, and E (reaction-2). The disparity of DNA sequences between the two groups precluded the design of a single PCR reaction covering all 51 serotypes with optimal specificity. As outlined in Table 1, PCR reaction-i includes three primer sets and three probes, and reaction-2 comprises a single primer set and two probes.

The strategy of combining a mixture of primers and probes in a single reaction was based on earlier results indicating that the presence of mismatches with the targeted sequences may result in decreased sensitivity of detection for certain AdV serotypes. Conversely, lower stringency conditions, including mainly a low temperature of the annealing step, as performed by other investigators in attempts to cover a broad spectrum of AdV serotypes (12), may compromise the specificity of the assay. The RQ-PCR assay presented is carried out under high stringency conditions, with an annealing temperature of 60° C., thus ensuring high specificity.

Additionally, more than 200 patient specimens were tested in parallel by the two-reaction RQ-PCR presented and the species-specific five-reaction RQ-PCR published previously (21). There was complete concordance between the results obtained, both for the AdV negative and positive findings. Under the stringent assay conditions used, no false positive results were observed in more than 1.000 clinical specimens investigated by the two-reaction assay, the majority of which reproducibly tested negative, as documented by Ct values of 50.

Sensitivity of the Assay

The lower detection limit of the diagnostic test described was assessed by analyzing dilution series prepared from reference strains of all 51 serotypes. The assay permitted reliable detection of 10² virus particles per ml of the medium investigated (FIG. 1). For reproducible quantification of virus load, however, the presence of ≧10³ particles per ml was necessary. When analyzing cell material, sensitivity of the assays permitted detection and quantification of virus copies at a level of 10 particles per 10⁶ cells. These results are equivalent to the five-reaction adenovirus assay developed previously (21), and are in line with other real-time PCR virus detection assays established (27).

Quantification of Virus Load

Virus particles in clinical samples were quantified according to standard curves established with reference strains of AdV serotypes 2 and 7, representative of species ACF and BDE, respectively (FIG. 1 and FIG. 2), as outlined in the Methods section. The two-reaction assay presented permitted analysis of virus copy numbers across a range of more than seven logs. In a series of more than 1.000 peripheral blood and stool samples derived from 45 paediatric patients after allogeneic stem cell transplantation, AdV positivity was detected in 11 (25%) of the individuals tested. The AdV species identified included-the subgenera A (1 case), C (9 cases) and D (1 case). The majority of AdV positive patients had a localised intestinal infection, as revealed by repeatedly positive stool samples. The AdV levels in patients who displayed self-limiting intestinal infection were below 5×10⁵ virus copies per gram of stool, without showing increasing viral load in serial samples. Three of the patients, who had higher virus levels in stool samples and showed rapid proliferation kinetics, experienced an invasive infection, as documented by subsequent detection of the virus in peripheral blood samples. This observation preceded the onset of clinical symptoms of disseminated AdV disease by more than a week in one of the patients, in agreement with the findings described earlier in a cohort of paediatric transplant recipients (21). This patient died from disseminated adenovirus disease, despite pre-emptive antiviral treatment. Two other patients with invasive AdV infection responded to pre-emptive antiviral therapy, which resulted in clearance of the virus from peripheral blood and in a dramatic decrease of viral load in serial stool samples. All AdV positive samples were re-tested using the species-specific RQ-PCR method described previously (21) and quantitative analysis revealed results highly concordant with the two-reaction RQ-PCR technique.

Concluding Comments

The two-reaction pan-adenovirus RQ-PCR assay described facilitates highly sensitive and specific detection of all currently known human serotypes of the virus, and permits quantitative assessment of viral load in a variety of clinical specimens. The assay has proved to be very well suited for high-throughput clinical screening. The performance of this assay is comparable to the species-specific RQ-PCR test established previously in our laboratory (21). The latter test, however, was too cost- and labour-intensive to permit its wide application in the clinical setting. Hence, the main advantages of the two-reaction assay presented include substantially lower costs with regard to reagents and labour, while the broad specificity and sensitivity have been maintained. The test permits rapid quantitative detection of any adenovirus serotype, which is an important prerequisite for timely onset of appropriate treatment. Wide application of the assay in clinical virus screening may therefore contribute to an improvement of the outcome of adenovirus infections in immunocompromised patients.

### REFERENCES

1. Akalu, A., W. Seidel, H. Liebermann, U. Bauer, and L. Dohner. 1998. J. Virol. Methods 71:187-196.
2. Baldwin, A., H. Kingman, M. Darville, A. B. Foot, D. Grier, J. M. Cornish, N. Goulden, A. Oakhill, D. H. Pamphilon, C. G. Steward, and D. I. Marks. 2000. Bone Marrow Transplant. 26:1333-1338.
3. Benko, M, Harrach, B, and Russelll, W C. Family Adenoviridae. Mastadenovirus. In: Van Regenmortel M H V, Fauquet C M, Bishop D K L, et al, eds. Virus Taxonomy: Seventh Report of the International Committee on Taxonomy of Viruses. New York: Academic Press; 1999: 227-238
4. Blanke, C., C. Clark, E. R. Broun, G. Tricot, I. Cunningham, K. Cornetta, A. Hedderman, and R. Hromas. 1995. Am. J. Med. 99:326-328.
5. Bruno, B., R. A. Zager, M. J. Boeckh, T. A. Gooley, D. H. Myerson, M. L. Huang, and R. C. Hackman. 2004. Transplantation 77:1049-1057.

6. Carrigan, D. R. 1997. Am. J. Med. 102:71-74.
7. Chakrabarti, S., K. E. Collingham, C. D. Fegan, D. Pillay, and D. W. Milligan. 2000. Bone Marrow Transplant. 26:305-307.
8. De Jong, J. C., A. G. Wermenbol, M. W. Verweij-Uijterwaal, K. W. Slaterus, P. Wertheim-Van Dillen, G. J. Van Doornum, S. H. Khoo, and J. C. Hierholzer. 1999. J. Clin. Microbiol. 37:3940-3945.
9. Echavarria, M., Forman, M., van Tol, M. J., Vossen, J. M., Charache,P. and Kroes, A. C. 2001. Lancet, 358: 384-385.
10. Flomenberg, P., V. Piaskowski, J. Harb, A. Segura, and J. T. Casper. 1996. J. Med. Virol. 48:267-272.
11. Hale, G. A., H. E. Heslop, R. A. Krance, M. A. Brenner, D. Jayawardene, D. K. Srivastava, and C. C. Patrick. 1999. Bone Marrow Transplant. 23:277-282.
12. Heim, A., C. Ebnet, G. Harste, and P. Pring-Akerblom. 2003. J. Med. Virol. 70:228-239.
13. Hierholzer, J. C. 1992. Clin. Microbiol. Rev. 5:262-274.
14. Hoffman, J. A., A. J. Shah, L. A. Ross, and N. Kapoor. 2001. Biol. Blood Marrow Transplant. 7:388-394.
15. Howard, D. S., G. L. Phillips II, D. E. Reece, R. K. Munn, J. Henslee-Downey, M. Pittard, M. Barker, and C. Pomeroy. 1999. Clin. Infect. Dis. 29:1494-1501.
16. Kidd, A. H., M. Jonsson, D. Garwicz, A. E. Kajon, A. G. Wermenbol, M. W. Verweij, and J. C. De Jong. 1996. J. Clin. Microbiol. 34:622-627.
17. Lankester, A. C., van Tol, M. J., Claas, E. C., Vossen, J. M. and Kroes, A. C. 2002. Clin. Infect. Dis., 34: 864-867.
18. La Rosa, A. M., R. E. Champlin, N. Mirza, J. Gajewski, S. Giralt, K. V. Rolston, I. Raad, K. Jacobson, D. Kontoyiannis, L. Elting, and E. Whimbey. 2001. Clin. Infect. Dis. 32:871-876.
19. Legrand, F., D. Berrebi, N. Houhou, F. Freymuth, A. Faye, M. Duval, J. F. Mougenot, M. Peuchmaur, and E. Vilmer. 2001. Bone Marrow Transplant. 27:621-626.
20. Lion, T., A. Gaiger, T. Henn, E. Horth, 0. A. Haas, K. Geissler, and H. Gadner. 1995. Leukemia 9:1353-1360.
21. Lion, T., R. Baumgartinger, F. Watzinger, S. Matthes-Martin, M. Suda, S. Preuner, B. Futterknecht, A. Lawitschka, C. Peters, U. Potschger, and H. Gadner. 2003. Blood 102:1114-1120.
22. Ljungman, P. 1997. Am. J. Med. 102:44-47.
23. Morris, D. J., G. Corbitt, A. S. Bailey, M. Newbould, E. Smith, S. Picton, and R. F. Stevens. 1993. J. Infect. 26:181-184.
24. Pring-Akerblom, P., F. E. Trijssenaar, T. Adrian, and H. Hoyer. 1999. J. Med. Virol. 58:87-92.
25. Runde, V., S. Ross, R. Trenschel, E. Lagemann, 0. Basu, K. Renzing-Kohler, U. W. Schaefer, M. Roggendorf, and E. Holler. 2001. Bone Marrow Transplant. 28:51-57.
26. Watzinger, F. and T. Lion. 1998. Leukemia 12:1984-1986.
27. Watzinger, F., Suda, M., Preuner, S., Baumgartinger, R., Ebner, K, Baskova, L., Niesters, H. G. M., Lawitschka, A., and Lion, T. 2004. Journal of Clin. Microbiol. (In Press)
28. Xu, W., M. C. McDonough, and D. D. Erdman. 2000. J. Clin. Microbiol. 38:4114-4120.
29. Leen A M, Sili U, Vanin E F, Jewell A M, Xie W, Vignali D, Piedra P A, Brenner M K, Rooney C M. 2004. Blood. 104: 2432-2440
30. Barik, S. 2004. Ann Med. 36:540-551
31. Joost Haarnoot, PC., Cupac, D., Berkhout, B. 2003. J Biomed Sci. 10:607-616
32. Roehr, B. 1998. J Int Assoc Physicians AIDS Care. 4:14-16

1-49. (canceled)

50. A nucleic acid molecule encoding an antigen or a fragment thereof comprising a nucleic acid sequence with at least 15 nucleotides having at least 80% sequence identity to at least a part of a nucleic acid molecule of any of SEQ ID NOs: 31 to 65.

51. The nucleic acid of claim 50, wherein the sequence identity is at least 90%.

52. The nucleic acid of claim 50, wherein the sequence identity is at least 95%.

53. The nucleic acid of claim 50, wherein the sequence identity is 100%.

54. The nucleic acid of claim 50, wherein the nucleic acid is DNA.

55. The nucleic acid of claim 50, wherein the nucleic acid is RNA.

56. The nucleic acid of claim 50, wherein the nucleic acid molecule is isolated from viral DNA or synthesized chemically.

57. The nucleic acid of claim 56, wherein the nucleic acid molecule is Adenovirus DNA.

58. An antigen comprising an amino acid sequence of any of SEQ ID NOs: 66 to 100.

59. The antigen of claim 58, wherein the antigen comprises amino acids 1 to 36 of SEQ ID NO: 66 to 100 (AdV serotypes 6, 8-10, 13-15, 18-20, 22-33, 36-39, 42-47, 49-51), amino acids 46 to 69 of SEQ ID NO: 66 to 75 or 77 to 100 (AdV serotypes 6, 8-10, 13-15, 18-20, 23-33, 36-39, 42-47, 49-51), amino acids 81 to 92 of SEQ ID NO: 66 to 75, 79, 81 to 94 or 98 to 100 (AdV serotypes 6, 8-10, 13-15, 18-20, 25, 27-33, 36-39, 42-44, 49-51), amino acids 102 to 225 of SEQ ID NO: 66 to 68, 70 to 75, 79, 81 to 94 or 98 to 100 (AdV serotypes 6, 8, 9, 13-15, 18-20, 25, 27-33, 36-39, 42-44, 49-51), amino acids 235 to 286 of SEQ ID NO: 66 to 75, 79, 81 to 94 or 98 to 100 (AdV serotypes 6, 8-10, 13-15, 18-20, 25, 27-33, 36-39, 42-44, 49-51), amino acids 327 to 368 of SEQ ID NO: 66 to 68, 70 to 75, 79, 81 to 94 or 98 to 100 (AdV serotypes 6, 8, 9, 13-15, 18-20, 25, 27-33, 36-39, 42-44, 49-51), amino acids 378 to 392 of SEQ ID NO: 66 to 68, 70 to 75, 79, 81 to 94 or 98 to 100 (AdV serotypes 6, 8, 9, 13-15, 18-20, 25, 27-33, 36-39, 42-44, 49-51), amino acids 414 to 472 of SEQ ID NO: 66 to 68, 70 to 75, 79, 81 to 94 or 98 to 100 (AdV serotypes 6, 8, 9, 13-15, 18-20, 25, 27-33, 36-39, 42-44, 49-51), amino acids 488 to 520 of SEQ ID NO: 66 to 68, 70 to 75, 79, 81 to 94 or 98 to 100 (AdV serotypes 6, 8, 9, 13-15, 18-20, 25, 27-33, 36-39, 42-44, 49-51), amino acids 536 to 572 of SEQ ID NO: 66 to 76, 78 to 100 (AdV serotypes 6, 8, 9, 10, 13-15, 18-20, 22, 24-33, 36-39, 42-47, 49-51), amino acids 582 to 637 of SEQ ID NO: 67, 69 to 81, 83 to 88 or 90 to 100 (AdV serotypes 8, 10, 13-15, 18-20, 22-27, 29-33, 36, 38, 39, 42-47, 49-51), amino acids 647 to 698 of SEQ ID NO: 67, 69 to 72, 74 to 81, 83 to 88 or 90 to 100 (AdV serotypes 8, 10, 13-15, 19-20, 22-27, 29-33, 36, 38, 39, 42-47, 49-51), amino acids 708 to 885 of SEQ ID NO: 69 to 72, 74 to 81, 83, 84, 87, 88, 90 to 98 or 100 (AdV serotypes 10, 13-15, 19-20, 22-27, 29-30, 32, 33, 36, 38, 39, 42-47, 49, 51), amino acids 895 to 903 of SEQ ID NO: 69 to 72, 74 to 81, 83, 84, 87, 88, 90 to 98 or 100 (AdV serotypes 10, 13-15, 19-20, 22-27, 29-30, 32, 33, 36, 38, 39, 42-47, 49, 51).

60. The antigen of claim 58, further defined as comprised in a pharmaceutical composition.

61. A method for treatment and/or prevention of a viral adenovirus infection comprising obtaining an antigen of claim 58 and administering the antigen to a subject.

62. An antibody directed towards an antigen of claim 58.

63. A method of conferring passive resistance to adenoviral infections comprising obtaining an antibody of claim 62 and administering the antibody to a subject.

64. A method of detecting an adenovirus in a sample comprising contacting the sample with an antibody of claim 62.

65. A method of detection of a single adenovirus serotype, an adenovirus subgenera, or an adenovirus in a sample comprising:
a) providing a sample comprising a nucleic acid;
b) contacting the sample with at least one forward primer and at least one reverse primer having at least 80% identity with a portion of a nucleotide sequence of a hexon gene derived from one of the sequences SEQ ID NO: 31 to 65; and
c) subjecting the sample contacted with the primers to a nucleic acid amplification technique.

66. The method of claim 65, further comprising determining the presence of a single adenovirus serotype, an adenovirus subgenera, or an adenovirus in the sample by detecting a nucleic acid amplification product.

67. The method of claim 65, wherein the nucleic acid amplification technique is performed with at least one oligonucleotide probe hybridizing in between the at least one forward primer and the at least one reverse primer within the hexon gene of an Adenovirus and derived from one of the sequences SEQ ID NOs: 31 to 65.

68. The method of claim 65, wherein the at least one oligonucleotide probe is tagged with a dye.

69. The method of claim 68, wherein the dye is a fluorescent dye.

70. The method of claim 68, wherein the at least one oligonucleotide probe is tagged with a quencher.


SEQUENCE LISTING (100 sequences)

SEQ ID NO: 1 - DNA, 392 bp, Artificial
  modified_base (12)..(387): n = a, c, g or t/u
atggcyacyccntcgatgmtgccscartggkcdtacatgcacatckcsggncaggaygcytcggagtacctgagyccsggbctggtgcarttygcccgygccacvgabacstacttcavyctgrrnaacaagtttagraaycccacngtrgcbccnacbcaygaygtnaccacvgaycgbwsncagcgnytraygytgcgnttyrtscchgtbgaycgbgargahamyrcntaykyntacaargykcgnthhacnctrgmygtrggngayaaymgngtdytdgayatggcywsywcntwctttgmyatymgvggbgtvytdgaymgnggycchasyttyaarccntaytcbggnacngchtayaaydshytngchcchaarrshgcncchaa

SEQ ID NO: 2 - DNA, 291 bp, Artificial
  modified_base (13)..(274): n = a, c, g or t/u
gcchaaymgrscnaaytayatygsnttyagrgayaayttyrtnggnytnwtgtaytayatayaywsyacngghaayatgggngtnytdgcnggncargcntcncarytnaaygcngtdgtngayytrcargayagraayacngarytdtchtaycarythhtrytdgaykcnhtdggngaymgyashmgdtayttywsywtgtggaayymnrcngtngayagytaygaycchgaygtnmgnrtyattgaraaycayggnryngargaygarytncchaaytaytgyttycc

SEQ ID NO: 3 - DNA, 1485 bp, Artificial
  modified_base (18)..(1482): n = a, c, g or t/u
gcymtggarathaayhtnvmggchaayytntggmrvaryttyytntaytcnaayrtdgsnytrtayytvcchgacdvnywvaarwahamnccvdnnaayrtvrmdhtdychrmhaaymmnaayachtaygastayatsaayrrbmgngtdrynbynccndnnbyngtngaydshtaymtnaaymtnggngcnmgntggtknyydgaybhnatgrayaayrtnaaycchttyaaycaycmmcghmaygcnggbytscghtaymgntchatgytnntrgghaayggnsgntwygtdccbttycayathcargtrccbcaraarttyttygcyrtnarraayytsytyctvytnccnggbtcntayacntaygartggaayttymgvaargaygtraayatgrtnctncaragywsnctngghaaygayctnmgvryngayggngchwssrtnmvbttymrnaryrtydvyctntaygchamhtthttbccbatggcncayaayacngcbtchacnytdgargchatgytnmgnaaygayaccaaygaycartcnttyaaygaytayytntsngsngsnaacatgytntaycchathccnkchaabgchacharyrtkccvathtchathcchtcncmvaaytgggcngchttymgvggntggdshytyacnmgnytnaarachaargarachccntcnytnggntcnggntwygayccntaytwyryntaytcbgghwcbrthccmtayytngayggnachttytayctnaaycayacyttyaaraargtnkcmvthaybttygactcytcngtbagytggccngghaaygacmgnytdytnwsnccnaaygarttygarathaarcgywshgtngayggsgarggntwyaaygtdrmhcartgyaayatgachaargaytggttyytnrtncaratgytndshmaytayaayatnggntaycarggyttyyayrtncchgarrghtayaargaymgnatgtaytcnttyttymgmaacttycagcchatgagymgncargtdgtnrayrmnryhrmntayamdgantwymarvmngtnrsdntnvhnywycarcayaayaaytcdggvttyrynggntachtngsnccbacywtrmgnsarggdsarscntancchgchaaykwbcchtayccncthathgghhmnacngcngtnvmnwsyvtnacncaraaaaarttyytntgygaymgnrynmtktggcgcathccvttytcbagyaayttyatgtcyatgggngcvstnacngayytbggncaraayhtkctntaygcmaaytcvgcbcaygcbytngahatrachttygargtbgayccmatggahgagccyacwctbctbtmtsttbtnttygaagkyttygacgtbgtvmgnrtncaycarccvcahmgcggmrtcatygarrcvgtvtayctvcgyacvccbttctcbgcbggnaacgchachacnwaa

SEQ ID NO: 4 - DNA, 1159 bp, Artificial
  modified_base (18)..(750): n = a, c, g or t/u
gcymtggarathaayhtnvmggchaayytntggmrvaryttyytntaytcnaayrtdgsnytrtayytvcchgacdvnywvaarwahamnccvdnnaayrtvrmdhtdychrmhaaymmnaayachtaygastayatsaayrrbmgngtdrynbynccndnnbyngtngaydshtaymtnaaymtnggngcnmgntggtknyydgaybhnatgrayaayrtnaaycchttyaaycaycmmcghmaygcnggbytscghtaymgntchatgytnntrgghaayggnsgntwygtdccbttycayathcargtrccbcaraarttyttygcyrtnarraayytsytyctvytnccnggbtcntayacntaygartggaayttymgvaargaygtraayatgrtnctncaragywsnctngghaaygayctnmgvryngayggngchwssrtnmvbttymrnaryrtydvyctntaygchamhtthttbccbatggcncayaayacngcbtchacnytdgargchatgytnmgnaaygayaccaaygaycartcnttyaaygaytayytntsngsngsnaacatgytntaycchathccnkchaabgchacharyrtkccvathtchathcchtcncmvaaytgggcngchttymgvggntggdshytyacnmgnytnaarachaargarachccntcnytnggntcnggntwygayccntaytwyryntaytcbgghwcbrthccmtayytnatggccactccstcgatgatgccgcagtggtcttacatgcayatcgccggtcaggaygcctcggartacctgagtcccggkctggtgcaattcgcccgygccacggacacctacttcaccctgggmaacaagtttagraaycccacygtggctcccacycatgaygtyaccaccgaycgytckcagcgkytgacrctgcgttttgtgcccgtggatcgggaagatactrcytaytchtacaaggctcgytttacgctrgctgtgggygayaaycgcgtrytwgayatggctagytcytactttgmyattmgaggggtrctrgaycgyggtcccagtttyagccbtattcbggwacsgcytayaaytctttrgcwccaaarggcgckccyaatgcywsmcartggyyrrm

SEQ ID NO: 5 - DNA, 1200 bp, Artificial
  modified_base (18)..(750): n = a, c, g or t/u
gcymtggarathaayhtnvmggchaayytntggmrvaryttyytntaytcnaayrtdgsnytrtayytvcchgacdvnywvaarwahamnccvdnnaayrtvrmdhtdychrmhaaymmnaayachtaygastayatsaayrrbmgngtdrynbynccndnnbyngtngaydshtaymtnaaymtnggngcnmgntggtknyydgaybhnatgrayaayrtnaaycchttyaaycaycmmcghmaygcnggbytscghtaymgntchatgytnntrgghaayggnsgntwygtdccbttycayathcargtrccbcaraarttyttygcyrtnarraayytsytyctvytnccnggbtcntayacntaygartggaayttymgvaargaygtraayatgrtnctncaragywsnctngghaaygayctnmgvryngayggngchwssrtnmvbttymrnaryrtydvyctntaygchamhtthttbccbatggcncayaayacngcbtchacnytdgargchatgytnmgnaaygayaccaaygaycartcnttyaaygaytayytntsngsngsnaacatgytntaycchathccnkchaabgchacharyrtkccvathtchathcchtcncmvaaytgggcngchttymgvggntggdshytyacnmgnytnaarachaargarachccntcnytnggntcnggntwygayccntaytwyryntaytcbgghwcbrthccmtayytnrhtstayasygargacgtaaacytdgarrybccwgacacrcayrttgtktwyaarccwrctgtyrchrmdgrmacwrtyrsttcdgaryysctvytrggwcarcaagcagcrccwaayagascmaaytacattgsmttcagagayaayttyatwggvctdatgtaytacaacagyacwggmaayatgggwgtrytkgcmggrcargcttcbcarctyaaygcagtrgtdgayctgcargayagraayacagagctgtcmtaccarytwatgytrgaygctttrggmgaymgmashcgktayttttcywtgtggaaytchgcmgtsgacagctacgatcctgacgtgcgaattattgaaaatcayggkgtrgargaygaactdccmaattaytgytttccdytwrgsgcwgtwggbrwwrtraawwmhtacaaaggyrtkaarymmratga

SEQ ID NO: 6 - DNA, 2275 bp, Artificial
  modified_base (18)..(750): n = a, c, g or t/u
gcymtggarathaayhtnvmggchaayytntggmrvaryttyytntaytcnaayrtdgsnytrtayytvcchgacdvnywvaarwahamnccvdnnaayrtvrmdhtdychrmhaaymmnaayachtaygastayatsaayrrbmgngtdrynbynccndnnbyngtngaydshtaymtnaaymtnggngcnmgntggtknyydgaybhnatgrayaayrtnaaycchttyaaycaycmmcghmaygcnggbytscghtaymgntchatgytnntrgghaayggnsgntwygtdccbttycayathcargtrccbcaraarttyttygcyrtnarraayytsytyctvytnccnggbtcntayacntaygartggaayttymgvaargaygtraayatgrtnctncaragywsnctngghaaygayctnmgvryngayggngchwssrtnmvbttymrnaryrtydvyctntaygchamhtthttbccbatggcncayaayacngcbtchacnytdgargchatgytnmgnaaygayaccaaygaycartcnttyaaygaytayytntsngsngsnaacatgytntaycchathccnkchaabgchacharyrtkccvathtchathcchtcncmvaaytgggcngchttymgvggntggdshytyacnmgnytnaarachaargarachccntcnytnggntcnggntwygayccntaytwyryntaytcbgghwcbrthccmtayytncavyrwmrswgawrmhaayvacwtagsmahwggkaayatmkyygccatggaaatkaattwcaggckaatytgtggmgaagyttyytgtaytcaaatgtgggsytrtacytrccwgacrrcttraaatacacycchggraacataaarctdccwgawaayaaraacacctacgagtacatgaayggscgygtkacwgyyccsggkytggtrgayacytaygtyaayatyggmgckcgytggtcbccwgaygthatggataayrtaaayccttttaaccaccaccgaaacgcagggytscgmtayagrtcyatgytrctrggcaayggbagrtttgtkccktttcacattcaggtrcckcaraaatttttygcyatharraayttgytgctkttrcchggytcstacacktacgartggaacttyagaaargatgtaaayatgatwcttcaragcacmctrggmaatgayctkmgrgtggayggrgcmagcrttcgctttgacaacatygcsctgtaygchaacttyttyccyatggcwcayaayacrgcktcyachytrgaagcyatgytrmgaaatgacaccaaygaccagtcwttyaaygaytayytrtgygckgcraacatgytgtayccyatyccagchaaygcaccagcgtgccrtttcwataccttcsmgaaattgggcrgcmttyagrggvtggagytttactcgcctwaaaactaargaracwccktchctkggytcwggstttgacccstactttgtdtactckggaaccattccctayytrgacggbachttttayctdaaycayactttyaaraaggtgtcaatyatgtttgactcctcygtkagytggccrggaaaygacmgkytgytwachccmaaygartttgaaatwaarcgttctgtrgayggggargghtacaaygtggcycaatgyaayatgacyaargaytggttyctwrtmcaratgctyagtcaytayaayattggrtaccarggtttytayrtkccagarrgctacaargaccgmatgtattctttcttyagaaactttcagccyatgagyagrcargttgtdgayacyacygartayaarrastayaaamaagtaacmgtrrarttycarcataacaaytcaggrttygtgggrtacytgggyccyacyatgcgsgarggwcaagcttayccmgchaactayccctayccycthatwggmmaracmgctgtrsaaagcrtmacrcaraaaaagtttctvtgygaycgygtdatgtggcgcatyccattttcyagyaayttyatgtcwatgggrgcsstwacvgayctyggrcaaaayatgctgtaygcaaaytcvgcccatgckytrgayatgacatttgargtkgayccmatggahgagccyacccttctttatgttttrtttgaagttttcgacgtggtrcgsattcaycarccrcaccgcggmgtcatygaagcggtmtayctrcgcacrccyttctcbgcgggtaacgcyaccacctaa

SEQ ID NO: 7 - DNA, 410 bp, Artificial
  Consensus sequence of serotype C AdVs hexon genes
atggctaccccttcgatgatgccgcagtggtcttacatgcacatctcgggccaggacgcctcggagtacctgagccccgggctggtgcagttygcccgcgccaccgagacgtacttcagcctgaataacaagtttagaaaccccacggtggcrcctacgcacgacgtraccacagaccggtcycagcgtttgacgctgcggttyatcccygtggaccgygaggatacygcrtactcgtacaargcgcggttyaccctrgctgtgggtgayaaccgtgtgctdgayatggcttccacrtactttgacatycgcggcgtgctggacmggggsccyacttttaagccctactcyggcactgcctacaacgchctrgcycccaarggygcycchaayycbtgygartgggawsa

SEQ ID NO: 8 - DNA, 1905 bp, Artificial
  Consensus sequence of serotype C AdVs hexon genes
gtwttgtacagygargatgtwrayatrgaaacyccwgayacwcaymtktcwtayawrcchrvhawdavkgawrrwaaykchmrrghhmtrhtkgghcaacaakcwatgccmaacagrcchaattacatygctttyagrgacaattttattggycthatgtattayaacagcacdggyaayatgggtgtyctkgckgghcargcatcrcagytraaygcygtkgtagayttgcargacagaaacacagagctktcmtaycarctyttgcttgattchatwggygayagaacmagrtayttttchatgtggaatcargctgtwgacagctatgatccagatgtyagaatyattgaraaycatggaactgargatgarytkccaaattaytgyttyccwctkgatgaaatargdgtkggwaayaaytttgccmtggaratyaayctdaatgccaacctrtggagaaatttcctbtactccaayatwgcrctgtayytgcchgacaagctaaartacavyccywcmaaygtrraaatwtctsmyaaccchaaywcmtacgaytayatgaacaagcgagtggtggctccygggytdgtrgaytgctacattaaccttggrgcrcgytggtchytkgactayatggacaacgtyaayccmtttaaccatcmccsatgckggcctvcgytaycctcmtgttgytgggmaayggycgctygtgcccttycaatycaggtkccycaraagttytttgccattaaraacctccthctcytgccrggctcatayacmtaygartggaacttcaggaargatgttaacatggtyctgcaragctcyctrggaaaygayctwagrgttgacggrgcyagcattaagtttgayagcatttgyctytacgccaccttyttyccsatggcccacaacacsgcctcmacgctkgargccatgctyagaaaygacaccaacgaccagtcctttaaygactaycthtccgccgccaacatgcthtayccyatacccgccaacgcyaccaacgtgcccatmtcyatcccmtcscgcaactgggcrgcwttycgmggytgggccttyacrcgyytkaagacwaaggaaaccccwtcmctggghtchggctacgacccttaytayacctactctggctcyataccmtacctwgayggaaccttytayctyaaycacaccttyaaraaggtrgccatyacytttgactcttctgtyagctggcckggcaaygaccgyctgcttacycccaaygagtttgaratyaarcgytcagttgacggrgagggytayaacgtwgcycartgyaacatgacraargactggttcytrgtrcaratgytrgcyaactmcaayatwggctaccagggcttytayatyccagaragctayaargaccgcatgtactcsttcttyagaaacttccagcccatgagccgkcargtggtkgaygatacyaaatacaargabtaycarcaggtkggmatymthcaccarcayaayaactcwggmttygtwggytacctbgcycccaccatgcgmgarggacargcytacccygcyaayktbccctayccrctwataggcaaraccgcrgttgayagyattacccagaaaaagtttctttgcgaycgcaccctktggcgcatyccmttctccagtaactttatgtccatgggygcrctcacagacctgggccaaaaccttctctaygcmaactccgcccacscgctagayatgacytttgaggtggatcccatggacgagcccacccttctttatgttttgtttgaagtctttgacgtggtccgkgtgcaccrgccgcaccgcggcgtcatcraraccgtgtacctgcgcacgcccttctcggcyggcaacgccacaacataa

SEQ ID NO: 9 - DNA, 1629 bp, Artificial
  Consensus sequence of serotype F AdVs Fiber genes
atgaaacgagccagayttgaagatgacttcaaccccgtctacccttacgaacactacaatccccttgacatyccatttattacacccccgtttgcytcctccaacggcttgcaagaaaaaccwccgggagtcctcagcctgaaatacactgatccacttacaaccaaaaacggggctttaaccttaaaaytgggcacgggactaaacattgatraaaatggagatctttcttcagatgctagcgtggaagttagcgcccctatyactaaaaccaacaaaatcgtaggtttaaattacactaarcctctygcyctgcraartaacgcgctyactctttcttacaacgcrcccttwaacgtagtaaataayaatttagctytaaatatstcacarcctgtyactrttaatgcaaacaacgaactttctctcttaatagacgccccacttaatgctgacacgggcactcttcgccttcraagtgmtgcacctcttggactagtrgacaaaacactaaargttttgttttctagccccctctatctagataataactttcttacactagccattgaacgcccgctagctctatccagtarcagagcagtgrcccttaagtattcaccacctttaaaaatagaaaacgaaaacttaaccctaagcacaggcggrccttttactgtaagygggggaaatytaaacytrrcaacatcggcaccyctctccgtgcaaaacaaytctctcccgccttttmycatcactgactctggattagctatggacttaggagacggtcttgcaytaggtggctctaagttaataatcaatcttggtccaggtttacaaatgtctaatggagctattactttagcactagatgcagcgctgcctttgcaatatararacaaccaacttcaactcagaattggctcmrcrtctgsyttaattatgagcggagtaacacaaacattaaacgtcaatgccaataccrgcaaaggtcttgctrttgaaaayaactcactagttgttaagctwggraacggtcttcgctttgatagctggggaagcatarctgtctcrcctactaccactacccctaccaccctatggaccaccgcrgacccrtcwcctaacgccactttttatgaatcactagacgccaaagtgtggctagttttagtaaaatgcaacggcatggttaacgggaccatatccattaaagctcaraaaggcaytttacttaraccyacagctagytttatttcctttgtcatgtatttytacagcgayggaacrtggagraaaaactatcccgtgtttgacaacgaagggatactagcaaacagtgccacrtggggttatcgacaaggacagtctgccaacactaacgtttcyaatgctgtagaatttatgcctagctctaaaagrtatcccaatsaaaaaggttctgaagttcagaacatggctcttacctacacttttttgcaaggtgaycctaacatggccatatcytttcagagyatttataatcatgcawtagaaggctactcattaaaattyacctggcgcgttcgaaataatgaacgttttgacatcccctgytgctcattttcttatgtaacagaacaataa

SEQ ID NO: 10 - DNA, 400 bp, Artificial
  modified_base (156)..(394): n = a, c, g or t/u
atggcyacccchtcgatgmtgccscartgggcrtacatgcacatcgccggrcaggaygcytcggagtacctgagyccgggtctggtgcagttygcccgygcmacmgacacstacttcarymtgggvaayaagtttagvaaycccacvgtrgcbccnacccacgatgtgaccacsgaccgbwsccagcgnctvabgytgcgcttygtgcccgtkgaycgsgargacamyacvtactcdtacaargykcgvthcacnctggcygtgggcgacaacmgvgtgctdgayatggccagcacntwctttgacatymgsggbgtsytkgaymgvggycchagyttcaarccmtaytcbggnacngcytayaaywsnytdgcyccyaarrghgcbcchaaynchwsty

SEQ ID NO: 11 - DNA, 420 bp, Artificial
  modified_base (6)..(420): n = a, c, g or t/u
scwranrtdrtnhtktayrcngaraayrynnryythsarrcncsdgayacncayrtdgtdtwyaarbcggrhrhnkmdrahdvnarytchnmnnyhaayytnnbdcarcarkchatgschaayagrccnaaytayatyggnttyagrgayaayttyrtnggnctnwtgtaytayaayagyactgghaayatgggngtdctrgcngghcargcntchcarytraaygcngtggtngacytrcargayagaaayachgarytdtchtaycarcthytrctwgaytcbytkggngayagracmmgrtarttragratgtggaayymnrcdgtngayagytaygaycchgaygtnmgnrtyattgaraaycayggbrtbgargaygarytkcchaaytaytgyttyccnytkrayggcndysrn

SEQ ID NO: 12 - DNA, 1484 bp, Artificial
  modified_base (28)..(1481): n = a, c, g or t/u
yatggaratcaaymtbcargcyaayctntggmrragtttyctdtaytcbaaygtdgsnytrtayytncchgaywhntwyaartacacnccrncyaayrtyambytbccmrmhaayammaacacytaygastacatgaayggbmgngtrgtdbcvccmtsnctrgtngaydchtayrtnaacatnggbgcsmgvtggtcnrtdgayschatgracaaygtnaayccmttyaaccaycamcgyaaygcdggyytrcgntacmgntccatgytbhtrgghaayggncgytaygtrcchttccayathcaagtkccycaraarttyttygcyrthaaraacctrctbctnctnccnggytcytacacytaygwgtggaayttymgvaaggaygtnaacatgrtnctrcaragttccctygghaaygayctvcgvryngayggygcywvsrtvmgyttyrmsagcrtyaaycthtaygcyachtthttsccyatggcncrcaayachgcytcmacbytkgaagccatgctgcgsaaygayaccmaygaycartcmttyaaygactayctrtchgcngcsaayatgctytayccmathccdgccaabgchacmaayrtkcchathtccatycchtcdcgcaactgggcvgcyttymgvggmtggwshttyacbmgnctsaaraccaargarackcchtcbytnggntcnggdttygayccmtacttygthtaytckgghtcbathccmtayctbgayggdaccttytayctnaaycacacyttyargaargtytmmatmaysttygactcytchgtbagctggcchgkhaaygacmgrytvytnwsnccnaaygarttygarathaagcgcashgtbgayggsgarggmtwcaaygtrrmhcaatgyaayatgaccaargactggttcytsgthcagatgctbtchmaytacaacathgghtaycagggcttyyayrtbcchgargghtacaargaycgcatgtaytcmttyttymgmaacttccagccyatgagyagvcaggtrgthgatgaggtyaaytacamdgaytwyaargcngtnrcyntrscmtwycarcayaayaactckggcttyrynggntacmtngcnccbacywtgmghcarggnsarscntaycchgcyaaytwyccmtayccnctnatygghhcmanshgcmgtnmmnwsygtyacscaraaaaarttyhtstgygayagrryyatgtggcgcathccvttctcbagcaacttcatgtcyatgggngcccthacvgacytggghcaraayhtgctntaygcyaactcvgcbcaygcdctngahatgacyttygargtsgayccmatggatgagcccaccctbctytmtcttstyttygaagtyttcgacgtggtcmgagtrcaycagccrcmhhgcggcrtcatygaggcmgtctacctgcgbacvccsttctcvgccggnaacgchacyacnwaa

SEQ ID NO: 13 - DNA, 18 bp, Artificial
  Primer
ggkctggtgcaattcgcc

SEQ ID NO: 14 - DNA, 20 bp, Artificial
  Primer
acctgggccaaaaccttctc

SEQ ID NO: 15 - DNA, 16 bp, Artificial
  Primer
acatgcacatcgccgg

SEQ ID NO: 16 - DNA, 17 bp, Artificial
  Primer
tcgatgatgccgcagtg

SEQ ID NO: 17 - DNA, 17 bp, Artificial
  Primer
cacgggcacaaaacgca

SEQ ID NO: 18 - DNA, 19 bp, Artificial
  Primer
cgtccatgggatccacctc

SEQ ID NO: 19 - DNA, 17 bp, Artificial
  Primer
cggtcsgtggtcacatc

SEQ ID NO: 20 - DNA, 18 bp, Artificial
  Primer
gagggtgaagtaggtgtc

SEQ ID NO: 21 - DNA, 18 bp, Artificial
  Primer
caggctgaagtaggtatc

SEQ ID NO: 22 - DNA, 20 bp, Artificial
  Primer
cccgtgtttgacaacgaagg

SEQ ID NO: 23 - DNA, 27 bp, Artificial
  Primer
ttagagctaggcataaattctacagca

SEQ ID NO: 24 - DNA, 25 bp, Artificial
  Probe
ccacggacacctacttcaccctggg

SEQ ID NO: 25 - DNA, 20 bp, Artificial
  Probe
aactccgcccacgcgctaga

SEQ ID NO: 26 - DNA, 15 bp, Artificial
  Primer
cgggtctggtgcagt

SEQ ID NO: 27 - DNA, 15 bp, Artificial
  Probe
cgggtttggtgcagt

SEQ ID NO: 28 - DNA, 15 bp, Artificial
  Probe
aggacgcctcggagt

SEQ ID NO: 29 - DNA, 15 bp, Artificial
  Probe
tcaggatgcctcgga

SEQ ID NO: 30 - DNA, 30 bp, Artificial
  Probe
atcgacaaggacagtctgccaacactaacg

SEQ ID NO: 31 - DNA, 2892 bp, Adenovirus serotype 6 Hexon gene
atggctaccccttcgatgatgccgcagtggtcttacatgcacatctcgggccaggacgcctcggagtacctgagccccgggctggtgcagttcgcccgcgccaccgagacgtacttcagcctgaataacaagtttagaaaccccacggtggcgcctacgcacgacgtgaccacagaccggtctcagcgtttgacgctgcggttcatccccgtggaccgcgaggatactgcgtactcgtacaaggcgcggttcaccctagctgtgggtgataaccgtgtgctagacatggcttccacgtactttgacatccgcggcgtgctggacaggggccctacttttaagccctactctggcactgcctacaacgcactggcccccaagggtgcccccaactcgtgcgagtgggaacaaaatgaaactgcacaagtggatgctcaagaacttgacgaagaggagaatgaagccaatgaagctcaggcgcgagaacaggaacaagctaagaaaacccatgtatatgcccaggctccactgtccggaataaaaataactaaagaaggtctacaaataggaactgccgacgccacagtagcaggtgccggcaaagaaattttcgcagacaaaacttttcaacctgaaccacaagtaggagaatctcaatggaacgaagcggatgccacagcagctggtggaagggttcttaaaaagacaactcccatgaaaccctgctatggctcatacgctagacccaccaattccaacggcggacagggcgttatggttgaacaaaatggtaaattggaaagtcaagtcgaaatgcaatttttttccacatccacaaatgccacaaatgaagttaacaatatacaaccaacagttgtattgtacagcgaagatgtaaacatggaaactccagatactcatctttcttataaacctaaaatgggggataaaaatgccaaagtcatgcttggacaacaagcaatgccaaacagaccaaattacattgcttttagagacaattttattggtctcatgtattacaacagcacaggtaacatgggtgtccttgctggtcaggcatcgcagttgaacgctgttgtagatttgcaagacagaaacacagagctgtcctaccagcttttgcttgattcaattggcgacagaacaagatacttttcaatgtggaatcaagctgttgacagctatgatccagatgtcagaattattgagaaccatggaactgaggatgagttgccaaattattgctttcctcttggtggaattgggattactgacacttttcaagctgttaaaacaactgctgctaacggggaccaaggcaatactacctggcaaaaagattcaacatttgcagaacgcaatgaaataggggtgggaaataactttgccatggaaattaacctgaatgccaacctatggagaaatttcctttactccaatattgcgctgtacctgccagacaagctaaaatacaaccccaccaatgtggaaatatctgacaaccccaacacctacgactacatgaacaagcgagtggtggctcctgggcttgtagactgctacattaaccttggggcgcgctggtctctggactacatggacaacgttaatccctttaaccacccccgccatgcgggcctgcgttaccgctccatgttgttgggaaacggccgctacgtgccctttcacattcaggtgccccaaaagttttttgccattaaaaacctcctcctcctgccaggctcatacacatatgaatggaacttcaggaaggatgttaacatggttctgcagagctctctgggaaacgaccttagagttgacggggctagcattaagtttgacagcatttgtctttacgccaccttcttccccatggcccacaacacggcctccacgctggaagccatgctcagaaatgacaccaacgaccagtcctttaatgactacctttccgccgccaacatgctatatcccatacccgccaacgccaccaacgtgcccatctccatcccatcgcgcaactgggcagcatttcgcggttgggccttcacacgcttgaagacaaaggaaaccccttccctgggatcaggctacgacccttactacacctactctggctccataccataccttgacggaaccttctatcttaatcacacctttaagaaggtggccattacttttgactcttctgttagctggccgggcaacgaccgcctgcttactcccaatgagtttgagattaagcgctcagttgacggggagggctataacgtagctcagtgcaacatgacaaaggactggttcctagtgcagatgttggccaactacaatattggctaccagggcttctacattccagaaagctacaaagaccgcatgtactcgttcttcagaaacttccagcccatgagccggcaagtggtggacgatactaaatacaaagattatcagcaggttggaattatccaccagcataacaactcaggcttcgtaggctacctcgctcccaccatgcgcgagggacaagcttaccccgctaatgttccctacccactaataggcaaaaccgcggttgatagtattacccagaaaaagtttctttgcgaccgcaccctgtggcgcatccccttctccagtaactttatgtccatgggtgcgctcacagacctgggccaaaaccttctctacgcaaactccgcccacgcgctagacatgacctttgaggtggatcccatggacgagcccacccttctttatgttttgtttgaagtctttgacgtggtccgtgtgcaccagccgcaccgcggcgtcatcgagaccgtgtacctgcgcacgcccttctcggccggcaacgccacaacataa

SEQ ID NO: 32 - DNA, 2829 bp, Adenovirus serotype 8 Hexon gene
atggccaccccctcgatgatgccgcagtgggcgtacatgcacatcgccgggcaggacgcctcggagtacctgagcccgggtctggtgcagtttgcccgtgccaccgacacgtacttcagcctgggcaacaagtttagaaaccccacggtggctcccacccacgatgtgaccacggaccggtcccagcgtctgacgctgcgctttgtgcccgtggatcgcgaggacaccacgtactcgtacaaggcgcgcttcactctggccgtgggcgacaaccgggtgctagacatggccagcacgtactttgacatccgcggcgtcctggaccgcggtcccagcttcaaaccctactcgggcacggcttacaacagcctggctcccaagggcgcccccaatcccagtcagtgggaacaggcaaaaacaggagctggtgtagaccaaaatcaaaaagaaactcgcacttacggcgtagccgccactggcggatataacattacaaaagaaggacttcagattggaattgatgaaactaaagaagatccaaacaacaaaatttatgcagataaaacattccagccggaaccacaaataggggaaaataattggcaggacactaatgttttctatggcggcagagctcttaaaaaggaaaccaagatgaaaccatgctatggctcatttgccagaccaactaataaaaaaggagggcaggctaaagttttaaccactgaagatggtcaacctacagaaaactttgacatagacttggctttctttgatattccacaagctggtggaaatgacaatcttgatccagatatgattctgtatgccgaaaatgtaaatctagaaacacctgacacacatgtagtttataaaccaggcaaggatgatgcaagttctgccgctaacctcacacaacagtccatgccaaacagacccaactacatcggtttcagggacaactttgtgggacttatgtattacaacagcactggcaacatgggtgtgctggctggtcaggcctctcagttaaatgctgtggtcgacttgcaagacagaaataccgagctgtcttatcagcttttattagattctctgggtgacaggaccagatactttagtatgtggaactctgcggtggacagctatgatccagatgtcaggatcattgagaatcacggtgtggaggacgaacttccaaattattgcttcccattggatggtaccggtaccaatgccacataccaaggtgtggaaccagataatgctcaaggtcaaaatgataaatggaaaaaggacgagaaagtggctgctcaaaaccaaatttgcaagggcaatatttatgccatggagattaacctccaggccaacctgtggaagagttttctgtactcgaacgtggccttgtacctgcccgactccttcaagtacacgccggccaacgtcacgctgcccactaacaccaacacctacgagtacatgaacgggcgcgtggcggccccctcgctggtggacgcctacgtcaacattggcgcccgctggtcactggaccccatggacaacgttaacccctttaaccaccaccgcaatgcgggcctgcgctaccgctccatgctgctgggcaacgggcgctacgtgcccttccatattcaagtgccccaaaagttttttgccattaagaacctgcttctgctcccgggctcctacacctacgagtggaacttccgcaaggatgttaacatgattctgcagagttcccttggcaacgacctgcgcgtcgacggtgcctccgtccgcttcgacagtgttaacctttacgccacctttttccccatggcgcacaacaccgcctccaccctggaagccatgctgcgcaacgacaccaacgaccagtccttcaacgactacctctcagccgccaacatgctttaccccatcccagccaaggccaccaacgtgcccatttccatcccctcgcgcaactgggccgcctttcgcggctggagtttcacccgcctaaaaaccaaggaaactccctccctcggctcgggttttgacccctactttgtttactcgggcaccatcccctacctcgacgggaccttctacctgaaccacacctttaagaaggtctccatcatgttcgactcctcggttagctggcccggcaacgacaggctgcttacgccgaacgagtttgaaattaagcgcagcgtcgacggggagggctataacgtggcccaatgcaacatgaccaaggactggttcctggtccagatgctttcccactacaacattggctaccagggcttccatgtgcctgagggctacaaggaccgcatgtactccttctttcgcaacttccagcccatgagtaggcaggtggttgatgagattaactacaaggattacaaggccgttgccctgcccttccagcacaacaactcgggcttcaccggctacctcgcacctaccttgcgccagggacagccctaccccgccaacttcccctacccgcttatcggacagaccgccgtgccctccgttacccagaaaaagtttctctgcgacagggtcatgtggcgcatccccttctccagcaacttcatgtctatgggcgcccttaccgacctgggtcaaaacatgctttacgccaactcggcgcacgcgctcgacatgacctttgaggtggaccccatggatgagcccaccctcctgtatcttctctttgaagttttcgacgtggtcagagtgcaccagccgcaccgcggcgtcatcgaggccgtctacctgcgcacgcccttctccgccggcaacgccaccacctaa

SEQ ID NO: 33 - DNA, 2835 bp, Adenovirus serotype 9  Hexon gene
atggccaccccctcgatgatgccgcagtgggcgtacatgcacatcgccgggcaggacacctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacagcttacaacagtctggcccccaagggtgcccccaactccagccagtggcttgcaaaagacaccaatgctggcgatcaagcattaaaaacccacacacatggcgtagctgctatggggggaacagatatcacagcaaagggtttgcaaattggtgttgacacgactgaaaacaagaatgagcctatttatgcaaatgaaatataccagccagaacctcaggtaggagaggaaaacttgcaagatgttgaaaacttttatggaggcagagctcttaaaaaagaaaccaaaatgaaaccttgctatggctcgtttgccagacccacaaatgaaaaaggcgggcaagccaaaatttttaacttgacggccattggtcagccaactaaaaatcatgatatcacaatgaatttctttgacactcctggaggaacagttggtcaggatactgaacttgaagcagacattgttatgtatgctgagaatgtgcatctggaaactccagacacgcatgtggtgtacaaaccaggaacttctgatgagagttcagaagcaaatttggttcagcagtccatgccaaacaggcccaactacatcggcttcagggacaactttgtgggtctcatgtactataacagcactggcaacatgggtgtgctggctggtcaagcatctcagttgaatgctgtggtcgacttgcaagacagaaacacagagctgtcttaccagctcttgctagattctctgggtgacagaaccagatactttagcatgtggaactctgcagtggacagttatgatcctgatgtcaggattattgaaaatcacggtgtggaagatgaacttccaaactattgcttcccattggatggagctggcactaatgctacctaccaaggtgtaaaagttaaaaatggccaagatggagatgtaaacgcagattgggaaaaagatccaaatcttgcttcacgaaaccaaatatgcaagggtaacatcttcgccatggagatcaacctccaggccaacctgtggaagagttttctgtactcgaatgtggccctgtacctgcccgactcatacaagtacacgccggccaacgtcacgctgcccgccaataccaacacctacgagtacatgaacggccgcgtggtagccccctcgctggtggacgcctacatcaacatcggcgcccggtggtcgctggaccccatggacaacgtcaacccattcaaccaccaccgcaacgcgggcctgcgttaccgctccatgcttatgggcaacggccgctacgtgcccttccacatccaagtgccccaaaagttctttgccatcaagaacctgctcctgctccccggctcctacacctacgagtggaacttccgcaaggatgtcaacatgatcctgcagagttccctcggaaacgacctgcgcgtcgacggcgcctccgtccgcttcgacagcgtcaacctctacgccacattcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcacccggctcaagaccaaagaaactccctccctcggctcgggtttcgatccctactttgtatactcgggttccatcccctacctcgacgggaccttctacctcaaccacaccttcaagaaggtctccatcatgttcgactcctcggtcagctggcccggcaacgaccggctgctcacgccgaacgagttcgagatcaagcgcagtgtcgacggggagggctacaatgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaacaactcgggcttcaccggctaccttgcacccaccatgcgtcaggggcagccctaccccgccaacttcccctatcctctcatcggccagacagccgtgccctctgtcacccagaaaaagttcctctgcgacagggtcatgtggcgcatccccttctccagcaacttcatgtccatgggcgccctcaccgacctgggtcagaacatgctctatgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacctaa

SEQ ID NO: 34 - DNA, 2850 bp, Adenovirus serotype 10 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacagcttacaacagcctggcccccaagagcgcccccaatccaagtcagtggaccgccaatgaaaaacaaactggcggtcaaccaaaatctgttacccaaacatttggatctgctccaatgggaggcagcaatattactattgaaggattggttattggaactaaggaggaagaaggcaatgccactgaagaaatattcgcagataaaacattccagccagaacctcaagtaggagaagaaaactggcaggaaacagaagccttctatggaggaagggctcttaaaaaggataccaaaatgaaaccatgctacggttcatttgctagacccaccaatgaaaaaggagggcaagcaaaattaaagctcaacgctcagggtcagccaactaaagattatgacatagacctggcattctttgatactccgggcggaacacctccaacaggcagtggtcaacaggaagaatacaaagcagacattattatgtacactgaaaatgtcaaccttgaaaccccagacacccacgtggtatataagccaggaaaagaggatgagagttctgaaacaaatttgacacagcagtccatgcccaacagacctaactacattggctttagggacaactttgtggggctcatgtattacaacagcaccggcaatatgggtgtgctggctggtcaggcttctcagttgaacgctgtggtcgacttgcaagacagaaataccgagctatcttaccagctattgctagattctttgggcgacaggaccagatactttagcatgtggaactctgcggtggacagctatgatcccgatgtcaggatcattgagaatcacggtgtggaagatgaacttccaaactattgcttcccattgaatggtactggcaccaattccacttatcaaggcgtaaaggtgaaaacaggtcaggatggagctgaggagaccgagtgggataaagatgaaactgttgcaagacaaaatcaaatcgccaagggcaacgtctatgccatggagatcaacctccaggccaacctgtggaagagttttctgtactcgaacgtagccctgtacctgcctgactcatacaagtacacgccggccaacgtcacgctgcccgccaataccaacacctacgagtacatgaacggccgcgtggtagccccctggctggtggacgcctacatcaacatgggggcccggtggtcgctggaccccatggacaacgtcaacccattcaaccaccaccgcaacgcgggcctgcgttaccgctccatgcttctgggcaacggccgctacgtgcccttccacatccaagtgccccaaaagttctttgccatcaagaacctgctcctgctccccggctcctacacctacgagtggaacttccgcaaggatgtcaacatgatcctgcagagttccctcggaaacgacctgcgcgtcgacggcgcctccgtccgcttcgacagcgtcaacctctacgccacattcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcacccggctcaagaccaaagaaactccctccctcggctcgggtttcgatccctactttgtatactcgggctccatcccctatctcgatgggaccttctacctcaaccacaccttcaagaaggtctccatcatgttcgactcctcggtcagctggcccggcaacgaccggctgctcacgccgaacgagttcgagatcaagcgcagtgtcgacggggagggctacaatgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaacaactcgggcttcaccggctacctcgcacccaccatgcgtcaggggcagccctatcccgccaacttcccctacccgctcatcggccagacagccgtgccctccgtcacccagaaaaagttcctctgcgacagggtgatgtggcgcatccccttctccagcaacttcatgtccatgggcgccctcaccgacctgggtcagaacatgctctacgccaactcggctcacgcgctcgaaatgaccttcgaggtggaccccatggatgagcccaccctcctctatcttctctttgaaggtttcgacgtggtcagagtgcaccagccgccaagcggcgtcatcgaggccgtctacctgcgcacgcccttctccgccggcaacgccaccacctaa

SEQ ID NO: 35 - DNA, 2820 bp, Adenovirus serotype 13 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacgtactttgacatccgcggcgtcctggaccgcggtcccagcttcaaaccctactcgggcacggcttacaacagcctggcccccaagggcgcccccaatcccagtcagtggactactaaagaaaagcagaccggagtaaatgcaggagacaaagaagttaccaagacatttggaattgccgccatgggaggcagtaatatttctgaaaacggtttgcagattggaactgacacaacagcagatggcacaaaaccaatatatgcagacaaaactttccagccagaacctcaagttggagaagaaaactggcaggataatgatgaatattatggaggcagggctcttaaaaaagataccaaaatgaagccatgctatggttcatttgctaaacccacaaacaaggaaggagggcaggctaaattgaaagaaacacccaatggcgctgatcctcaatatgatgtggacatggctttctttgactcaacaactataaacattccagatgtagtgttgtacactgaaaatgtagacttggaaactccagacacacatgtggtgtacaaaccaggcaaagaggatgacagttcagaagtaaacttaactcagcagtccatgcctaacagacccaactacattggcttcagagacaactttgtggggctcatgtactacaacagcactggcaacatgggtgtgctggctggtcaggcttctcagttgaatgctgtggttgacttgcaagacagaaacaccgaactgtcttaccagctcttgctagattctctgggtgacagaacaagatactttagcatgtggaactctgcggtggacagttatgatcccgatgtcaggatcattgaaaatcacggtgtggaagatgaacttccaaactactgcttccccttggacggtattcaaactaattcagcctaccaaggtgttaaactaaaggataatccaacaggaggcggagctaatggagattgggtaaaggatgataccatttcagcccataatcaaattgggaagggcaacatctttgccatggagatcaacctccaggccaacctctggaagagttttctgtactcgaacgtggccttgtacctgcccgactcctacaagtacacgccggccaacgtcacgctgcccgccaacaccaacacctacgagtacatgaacggccgcgtggtagccccctcgctggtggacgcctacatcaacatcggcgcccgctggtcgttggaccccatggacaacgtcaaccccttcaaccaccaccgcaacgcgggcctgcgctaccgctccatgcttctgggcaacggccgctacgtgcccttccacatccaagtgccccaaaagttctttgccatcaagaacctgctcctgcttcccggctcctacacctacgagtggaacttccgcaaggatgtcaacatgatcctgcagagttccctcggcaacgacctgcgcgtcgacggcgcctccgtccgcttcgacagcgtcaacctctacgccaccttcttccccatggcgcacaacaccgcctccaccctggaagccatgctgcgcaacgacaccaacgaccagtccttcaacgactacctctcggccggcaacatgctctaccccatcccggccaaggccaccaacgtgcccatctccatcccctcgcgcaactgggccgccttccgcggctggagtttcactcgtctgaagaccaaggaaactccctctctcggttcgggtttcgacccctactttgtctactcgggctccattccctacctcgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgaaatcaactacaaggactacaaggccgtcaccctgcccttccagcacaacaactcgggcttcaccggctacctggcccccaccatgcgtcaggggcagccctaccccgccaacttcccctacccgctcatcggctccaccgcagtcccctccgtcacccagaaaaagttcctctgcgacagggtcatgtggcgcatccccttctccagcaacttcatgtccatgggcgccctcaccgacctgggtcagaacatgctctatgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacctaa

SEQ ID NO: 36 - DNA, 2838 bp, Adenovirus serotype 14 Hexon gene
atggccaccccatcgatgctgccccagtgggcatacatgcacatcgccggacaggatgcttcggagtacctgagtccgggtctggtgcagttcgcccgcgccacagacacctacttcaatctgggaaataagtttagaaatcccaccgtagcgccaacccacgatgtgaccaccgaccgtagccagcggctcatgttgcgcttcgtgcccgttgaccgggaggacaatacatactcttacaaagtgcggtacaccctggccgtgggcgacaacagagtgctggatatggccagcacgttctttgacattaggggcgtgttggacagaggtcccagtttcaaaccctattctggtacggcttacaactctctggctcctaaaggcgctccaaatgcatctcagtggttggataaaggggttgaaactactgaagaacggcaaaatgaagacggggaaaatgacgaaaaagctacatacacttttggcaatgccccagtaaaagccgatgctgacattacaaaagacggactaccaataggtttggaagtcccagctgaaggtgaccctaaacctatctacgctaataagctttaccaaccagaaccccaggtgggacaggaatcgtggactgatacagatggcactgaagaaaaatacggaggcagagtacttaaaccggacactaaaatgaaaccgtgctatgggtcttttgctaaacctactaatgtgaaaggcggacaggcaaaagtgaaaacagaagaagcaggcaacattgaatatgacattgacatgaacttttttgacttaagatcacaaaaacaaggtcttaaacctaagattgtaatgtatgcagaaaatgtggacctggaatctccagatactcatgttgtgtacaaacctgaagtttcagatgctagttcaaatgctaatcttggacagcagtctatgcccaacagacccaactacattggcttcagagataattttattggtcttatgtactataacagtactggtaacatgggggtgctggctggccaagcatctcagttgaatgcagtggttgacttgcaggacagaaacacagaactgtcttaccaactcttgcttgactccctgggcgatagaaccagatactttagcatgtggaatcaggctgttgacagttatgatcccgatgtgcgtgttattgaaaatcatggtgtggaagatgaacttcccaactactgttttccactggacggcatcggtccgcgaacagatagttacaaggagattcagttaaatggagaccaagcttggaaagatgtaaatccaaatggtatcagtgaacttgttaagggaaatccatttgccatggaaattaaccttcaagccaatctatggcgaagtttcctttattccaatgtggctctgtatctcccagactcgtacaaatacaccccgtccaatgtcactcttccagaaaacaaaaacacctacgactacatgaacgggcgggtggtgccgccatctctagtagacacctatgtgaacattggcgccaggtggtctctggatgctatggacaatgtcaacccattcaaccaccaccgtaacgctggcttgcgttaccgatccatgcttttgggtaacggacgttatgtgcctttccacatacaagtgcctcaaaaattcttcgctgtcaaaaacctgctgcttctcccaggctcctacacttatgagtggaacttcaggaaggatgtgaacatggtgctacagagttccctcggtaacgacctacgggtagatggcgccagcatcagtttcacgagcatcaacctctatgctacctttttccccatggctcacaacaccgcttccacccttgaagccatgctgcggaatgacaccaatgatcagtcattcaacgactatctatctgcagctaacatgctctatcccattcctgccaatgcaaccaatattcccatttccattccttctcgcaactgggcggctttcagaggctggtcatttaccagactcaaaaccaaagaaactccctctttggggtctggatttgacccctactttgtctattctggttctattccctacctggatggtaccttctacctgaaccacacttttaagaaggtttccatcatgtttgactcttcagtgagctggcctggaaatgacaggttactatctcccaacgaatttgaaataaagcgcactgtggatggcgaaggctacaatgtagcccaatgcaacatgaccaaagactggttcttggtacagatgctcgccaactacaacataggctatcagggcttctacattccagaaggatacaaagatcgcatgtattcatttttcagaaacttccagcccatgagcaggcaggtggttgatgaggtcaattacaaagacttcaaggccgtcgccataccctaccaacacaacaactctggctttgtgggttacatggctccgaccatgcgccaaggtcaaccctatcccgctaactatccctatccactcattggaacaactgccgtaaatagtgttacgcagaaaaagttcttgtgtgacagaaccatgtggcgcataccgttctcgagcaacttcatgtctatgggggcccttacagacttgggacagaacatgctttatgccaactcagctcatgctctggacatgacctttgaggtggatcccatggatgagcccaccctgctttatcttctcttcgaagttttcgacgtggtcagagtgcatcagccacatcgcggcatcatcgagacagtctacctgcgtacaccgttctcggccggtaacgctaccacgtaa

SEQ ID NO: 37 - DNA, 2826 bp, Adenovirus serotype 15 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcctacaacagtctggcccccaagggcgcccccaactccagtcagtgggaacagaaaaaggccaatgctggagatcaaaaggaaacacatacttatggtgtagctcctatgggtggagaaaacattacaattagcggtttgcaaattggaacagatactacaaatggcaagcaagacccgatatatgctaataagctgtatcaaccagagcctcaagtaggagaagaaaactggcaggaaacagaagccttctatggaggaagggctcttaaaaaggaaaccaagatgaaaccatgctatggctcatttgccagacccacaaatgaaaaaggaggacaggcaaaactaagagaccctgaaaaaagtcaagaagattttgacatagtgctagcattctttgatactccgggaggaactttaacaggtggtggaacggaatacaaagcagacattgttatgtgcactgaaaatgttaatcttgaaacccgggacacccacgtggtgtataaaccaggcaaagatgatgacagttcagaaatcaacttggttcagcagtccatgcccaacagacctaactacatcggcttcagggacaactttgtgggtctcatgtactacaacagcactggcaacatgggtgtgctggccggtcaggcttctcagttgaatgctgtggtcgacttgcaagacagaaacacagagctgtcttaccagctcttgctagattctctgggcgacagaaccaggtactttagcatgtggaactctgcggtggacagctatgatcccgatgtcaggatcattgagaatcacggtgtggaagatgaacttcccaactattgcttcccattggatgggtctggcaccaatgctgcttatgaaggtgtaaaagttaaaaatggagaagatggggatcaagagagcgaatgggaaaaagacaccaatgtggcagatcgaaaccaaatatgcaagggcaacatctacgccatggagatcaacctccaggccaacctgtggaagagttttctgtactcgaatgtggccctgtacctgcctgactcctacaagtacacgccggccaacgtcacgctgcccgccaacaccaacacctacgagtacatgaacggccgcgtggtagccccctcgctggtggacgcctacatcaacatcggcgcccgctggtcgttggaccccatggacaacgtcaaccccttcaaccaccaccgcaatgcgggcctgcgctaccgctccatgcttctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggaaacgatctgcgcgtcgacggcgcctccgtccgcttcgacagcgtcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcacccggctcaagaccaaggaaactccctccctcggctcgggtttcgacccctactttgtctactcgggctccatcccctatctcgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaatgtggcccaatgcaacatgaccaaggactggttcctcgtccagatgctctcccactacaacatcggttaccagggcttccatgtgcccgagggctacaaggaccgcatgtactccttcttccgcaacttccagcccatgagcaggcaggtggtcgatgagatcaactacaaggactacaaggccgtcaccctgcccttccagcacaacaactctggcttcaccggctacctcgcacccaccatgcgtcaggggcagccctaccccgccaacttcccctacccgctcatcggccagacagccgtgccatccgtcacccagaaaaagttcctctgcgacagggtcatgtggcgcatccccttctccagcaacttcatgtccatgggcgcccttaccgacctgggtcagaacatgctctacgccaactcggcccacgcgctcgacatgacctttgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacctaa

SEQ ID NO: 38 - DNA, 2760 bp, Adenovirus serotype 18 Hexon gene
atggccactccgtcgatgatgccgcagtggtcttacatgcatatcgccggtcaggatgcctcggaatacctgagtcccgggctggtgcaattcgcccgtgccacggacacctacttcaccctgggcaacaagtttagaaatcccactgtagctcccactcatgacgttaccaccgatcgttctcagcggctgacactgcgttttgtgcccgtggatcgggaagatactgcttactcatacaaggctcgttttacgctagctgtgggtgataaccgcgtacttgatatggctagttcctactttgacattagaggggtactagaccgcggtcccagtttcaagccgtattctggaacggcttacaactctttggctccaaagggcgcgcccaatgctagccagtggctgactactaatgacaacaagtcccacacttttgctcaagcgccttacatagggagctccatcactaaagatggcattcaagtgggaacaaataccgccaatcccccacaaccagtatatgccgacaagacatatcaacccgaacctcaagtaggggagagtcaatggaacgcgcctttgcctaacaacgcaaaagcagcaggaagggtgctcaagaacaccacacccatgtacccctgctatgggtcttacgcccgagccacaaatgaaaacggaggacaatcaaaaaataacgaaggggtggaaatgcaattttttgcatccgctgctgacaatcaaaatgatcccagtgttgtgctgtacagtgaggacgtaaacctggaagcccctgacacgcacattgtttttaagccagctgtagcagcagacactgttagttcggaactgctactaggtcaacaagcagcgcctaacagacccaattacattggcttcagagataacttcataggactaatgtactacaacagcactggaaatatgggagtgcttgccggacaggcttcgcagcttaatgcagtggtggacctgcaggacaggaatacagagctgtcctaccaacttatgctagacgctttaggcgaccgcagtcggtatttttctatgtggaactcagccgtggacagctacgatcctgatgtgcgaattattgaaaatcatggggtggaagacgaacttcccaattactgctttccactaggcgctgtggggattgtgaattcctacaaaggtgttaaatacgatgcaaatgcctgggcaaaggataacaacaaagcagataataataacttagcaacaggtaacatcttcgccatggaaataaatttgcaggctaatctgtggcgaagtttcttgtactcaaatgtgggcttgtacttacctgacagcttaaaatacactccgggaaacataaagctgcctgataataaaaacacctacgagtacatgaatggccgtgttacagccccggggttggtggatacttatgttaacatcggagctcgttggtcgccagatgttatggataacataaatccttttaaccaccaccgaaacgcagggctccgatacagatctatgctactaggcaatggcaggtttgtgccttttcacattcaggtgccgcagaaattttttgccataagaaacttgctgcttttacctggttcgtacacgtacgaatggaactttagaaaagatgtaaatatgatacttcaaagcaccctaggcaatgacctgcgggtggatggggcaagcattcgctttgacaacatcgcgctgtacgccaactttttccctatggctcataatacggcgtccaccctagaagctatgttgagaaatgacaccaacgaccagtcatttaatgactacctatgcgctgcaaacatgctgtaccctatcccagccaacgccaccagcgtgccgatttctataccttccagaaattgggcagcatttaggggatggagttttactcgcttaaaaactaaggaaacaccgtcactgggctctggctttgacccgtactttgtgtactcgggaaccattccctatttggacggtacattttatcttaatcatactttcaaaaaggtgtcaattatgtttgactcctctgttagctggccgggaaacgaccgtttgcttacgcccaacgagtttgaaattaagcgttctgtggatggggaagggtacaacgtggctcaatgcaacatgactaaagactggtttcttatccagatgcttagtcactacaacattgggtaccagggtttctatgtgccagaaggctacaaagaccgaatgtattctttcttcagaaactttcagcctatgagtagacaggttgttgacactactgagtacaaagactacaaacaagtaacagtgaagtttcaacataacaattcagggttcgtgggctacttgggtcccaccatgcgggaaggtcaagcttatccagctaactatccctatcccctaattggaaagaccgctgtggaaagcataacgcagaaaaagtttctgtgcgatcgcgtaatgtggcgcatcccattttccagcaactttatgtctatgggagcgcttaccgaccttggacaaaacatgctgtatgcaaattccgcccatgctttggacatgacatttgaggtggatccaatggacgagcccacccttctttatgttttgtttgaagttttcgacgtggtgcggattcatcagccgcaccgcggcgtcatcgaagcggtatacctacgcacgcctttctctgcgggtaacgctaccacctaa

SEQ ID NO: 39 - DNA, 2856 bp, Adenovirus serotype 19 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttagaaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcttacaacagcctggcccccaagggcgcccccaactccagtcagtgggatgctcaagaaaaaaatggacaaggaggaaatgacatggttaccaaaactcacacatttggtgtggctgctatgggaggaacaaatattacaaaccagggtttgttaattggaactgaagaaacagcccataatcctccaaaggaaatctttgcagacaaattattccagccagaacctcaagtaggagaggaaaactggcaagacaccaatgcattttatggaggcagggctcttaagaaggaaactaaaatgaaaccatgctatggatcttatgctagaccaacaaacacaagtggcggacaggctaagcttaaaactggtgacaatatcgatcctaccaaggatttcgacatagatcttgctttcttcgatactcctggcggaaatcctccagcaggtggtagtggaacggaagaatacaaagcagacattgttatgtacactgaaaatgtcaaccttgaaacacctgacactcatgtggtgtacaaaccaggcaaagaggatgaaagttctgaggccaacttggttcagcagtccatgcccaacagacccaactacattggcttcagagacaattttgtggggctcatgtattacaacagcactggcaacatgggtgtgctggctggtcaggcctctcagttgaatgctgtggtggacttgcaagacagaaacacagagctgtcttaccagctcttgctagattctctgggcgacagaaccaggtactttagcatgtggaactctgcggtggacagctatgatcccgacgtcaggatcattgaaaatcacggtgtggaagatgaacttccaaactattgctttccattggatggctctggtaccaatgctgcctaccaaggtgtaaaggttcaagatggtgaagacggggataaagaaactgaatgggaaaaagataccaaagtcgcagatcgtaaccaactgtgcaagggtaacatctttgccatggagatcaacctccaggccaacctgtggaagagttttctgtactcgaacgtggccttgtacctgcccgactcctacaagtacacgccggccaacgtcacgctgcccgccaacaccaacacctacgagtacatgaacggccgcgtggtagccccctcgctggtggacgcatacgtcaacatcggtgcgcgctggtcgctggaccccatggacaacgtcaaccccttcaaccaccaccgcaacgctggcctgcgctaccgctccatgcttctgggcaacggccgctacgtgcccttccacatccaagtgccccaaaagttctttgccatcaagaacctgctcctgctccccggctcctacacctacgagtggaacttccgcaaggatgtcaacatgatcctgcagagttccctcggaaacgacctgcgcgttgacggcgcctccgtgcgcttcgacagcgttaacctctacgccacattcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgaggctggagttttacccggctcaagaccaaggaaactccctccctcggctcgggtttcgacccctactttgtatactctggctccatcccctatctcgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaatgtggcccaatgcaacatgaccaaggactggttcctcgtccagatgctctcccactacaacatcggctaccagggcttccatgtgcccgagggctacaaggaccgcatgtactccttcttccgcaacttccagcccatgagcaggcaggtggtcgatgagatcaactacaaggactacaaggccgtcaccctgcccttccagcacaacaactcgggcttcaccggctacctagcacccaccatgcgtcaggggcagccctaccccgccaacttcccctacccgctcattggctccaccgcagtgccctccgtcacccagaaaaagttcctctgcgacagggtcatgtggcgcatccccttctccagcaactttatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacataa

SEQ ID NO: 40 - DNA, 2832 bp, Adenovirus serotype 20 Hexon gene
atggccaccccctcgatgatgccgcagtgggcgtacatgcacatcgccgggcaggacgcctcggagtacctgagcccgggtttggtgcagtttgcccgcgccaccgacacgtacttcagcctgggcaacaagtttaggaaccccacggtggctcccacccacgatgtgaccacggaccggtcccagcgtctgacgctgcgctttgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcctacaacagcctggctcccaagggcgcccccaactccagtcagtggcttgcaaaagataccaatgctgctggtcaaccagataaaactcacacctatggcgttgcagccatgggtggagaagacattacagaaaagggtcttcaaattggcattgatgaaactaaagaagaaaataacaagatatttgccaatgaaatataccaaccagaacctcacgtgggagaagaaaactggcaggaaacatttgttttttatggcggtagagctcttaagaaggataccaaaatgaaaccctgctttggctcatttgccagaccttattatgaaaagggaggtcaggctaaatttgtacttgatcaggaaggaaagccaactaaaaatcatgacatcacaatggctttctttgatactcctggtgggcaattaaatggaaaagatgaacttaaggcagacattgtcatgtacactgaaaatgtcaacctggaaacacctgacacccatgttgtttacaaacctggaaaagaggatgacagttcagaaatcaatttggttcaacagtccatgcccaacagacccaactacattggcttcagggacaactttgtagggcttatgtattacaacagcactggcaacatgggtgtgctggcaggtcaggcctctcagttgaatgctgtggtagacttgcaagacagaaacacagagctgtcttaccagctcttgctagattcgctgggtgacagaaccagatactttagcatgtggaactctgcggtggacagctatgatccagatgtcaggatcattgagaatcatggtgtggaagatgaacttccaaactattgcttcccattggatggtgctggaactaatgcagtttaccaaggtgtaaaaattacagatggaaatgatggcgatgtcaatgatgactgggaaaaagacaccgcagtatctgaacgtaatcagatatgcaaaggcaatatctatgctatggagatcaacctccaggccaacctgtggaagagttttctgtactcgaacgtgggcctgtacctgcccgactcctacaagtacacgccggccaacgtcacgctgcccgccaacaccaacacctacgagtacatgaacggccgcgtggtagccccctcgctagtggacgcctacatcaacatcggcgcccgctggtcgctagaccccatggacaatgtcaaccccttcaaccaccaccgcaatgcgggcctgcgctaccgttccatgcttctgggcaatggccgctacgtgcccttccacatccaagtgccccaaaagttctttgccatcaagaacctgctcctgctccccggctcttacacctacgagtggaactttcgcaaggacgtcaacatgatcctgcagagttccctcggaaacgacctgcgcgtcgacggcgcctccgtccgcttcgacagcgtcaacctctacgccaccttcttccccatggcgcacaataccgcctccaccctggaagccatgctgcgcaacgacaccaacgaccagtccttcaacgactacctctcggcagccaacatgctttaccccatcccggccaaggccaccaacgtgcccatttccatcccctcgcgcaactgggccgccttccgcggctggagtttcacccggctcaagaccaaggaaactccatcccttggctcgggtttcgacccctactttgtctactcgggctccatcccctacctcgacgggaccttctacctcaaccacaccttcaaaaaggtctccatcatgtttgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccattacaacatcggttaccagggcttccatgtgcccgagggctacaaggaccgcatgtactccttcttccgcaacttccagcccatgagcaggcaggtggtcgatgagatcaactacaaggactacaaggccgtcaccctgccattccagcacaacaactctggcttcaccggctacctcgcacccaccatgcgtcaggggcagccctaccccgccaacttcccctacccgctcatcggccagacagccgtgccatccgtcacccagaaaaagttcctctgcgacagagtcatgtggcgcatccccttctccagcaacttcatgtccatgggcgcccttaccgacctgggtcagaacatgctctacgccaactcggcccacgcgctcgacatgacctttgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacctaa

SEQ ID NO: 41 - DNA, 2808 bp, Adenovirus serotype 22 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcttacaacagcctggcccccaagggcgcccctaactccagtcagtgggcgcagaaaaagactggtgaagacaatcaaactgaaacacgcacatttggtgtggccgctatgggtggaatacttattgataaaaatggtcttcagattggaacagatgaaactaaacccaataacaaggaagtttatgcagacaaaacattccagccagaacctcaaataggtgaagaaaactggcaagatggagatgttttctatggaggcaggactattaaaaaggaaaccaaaatgaagccatgctatggctcatttgccagacccactaatgaaaagggaggtcaggcaaaatttaaaactaatgccgaaggtcagcccacagaggagttagacattgacctgaacttctttgatattaatggaggggcaggtgataatgaatttaacccagacatggtcatgtatgctgagaatatgaatctggagacgccagatacacatgtggtgtacaaacctggaacttcagatgacagttctgaagctaacttagcgcagcagtccatgcccaacaggccaaactacattggcttcagagacaattttgtggggcttatgtactacaacagcactggcaacatgggtgtgctggctggtcaggcatctcagttgaatgctgtggtcgacttgcaagacagaaatactgagctgtcttaccagctcttgctagattctctgggtgacagaacaagatactttagcatgtggaactctgcggtggacagctatgatcccgatgtcaggatcattgagaatcacggtgtggaagatgaacttccaaactattgcttcccattggatggcactggcaccaattctacataccaaggggttaaagaaacagctgctcagaatggatgggaaaaagatccaaatgttgctgctcagaaccaaatttgcaagggcaacatctatgccatggagattaacctccaggccaacctgtggaagagttttctgtactcgaacgtggccctgtacctgcccgactctttcaagtacacgccggccaacgtcactctgcccaccaacaccaacacctacgagtacatgaacggccgcgtggtggccccctcgctggtggacgcctacgtcaacatcggcgcccgctggtcgttggaccccatggacaacgtcaatcccttcaaccaccaccgcaacgcgggcctgcgctaccgctccatgctgctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggcaacgacctgcgcgtcgacggcgcctccgtccgctttgacagcgtcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcctatctccatcccctcgcgcaactgggccgccttccgcggctggagtttcacccggctcaagaccaaggaaactccctccctcggctcgggtttcgacccctactttgtctactcgggctccatcccctaccttgacgggaccttctaccttaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggttaccagggcttccatgtgcccgagggctacaaggaccgcatgtactccttcttccgcaacttccagcccatgagcaggcaggtggtcgatgagattaactacaaggactacaaggccgtcaccctgcccttccagcacaacaactcgggcttcaccggctacctcgcacccaccatgcgccaggggcagccctaccccgccaacttcccctacccgctcatcggccagacagccgtgccctccgtcacccagaaaaagttcctctgcgacagggtcatgtggcgcatccccttctccagcaacttcatgtccatgggcgcccttaccgacctgggtcagaacatgctctacgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacctaa

SEQ ID NO: 42 - DNA, 2796 bp, Adenovirus serotype 23 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggccccagcttcaaaccctactcgggcacggcttacaacagcctggcccccaagggcgcccccaatccaagtcagtgggaacagaaaaagactggtgttggaccagaagctatggaaaaacacacatttggaatggcagccatggctggagaagcaattactaacaagggactgcaaataggagtagataccacagatggtaaacaagacccaatatatgcaaatcagctgtatcagccagagcctcaagtgggggaagacagctggaatgatgatgttgcaccatcatatggcggtagggctctgaaaaaggaaaccaaaatgaaaccttgctatggctcgttcgccagacccactaatgaaaagggcggacaagctaaactcagagaccctgaaaaaagtcaagaagattttgacattgacatggctttctttgattccaacactattaacacaccagatgttgtgctgtatacagaaaatgtaaatttggaaactccagattcccatgttgtttacaaagcaggaacttcagacgaaagctctgaaattaatttgggtcaacagtcaatgccaaacaggccaaactacattggcttcagagacaactttgtggggctaatgtattacaacagcactggaaacatgggtgtgctggccggtcaagcctctcagttgaatgccgtggtcgacttgcaagacagaaacaccgagctgtcttaccagctcttgctagattctttgggtgacagaaccagatactttagcatgtggaactccgcggtagacagctatgatcccgatgtcaggatcattgaaaatcacggtgtggaagatgaacttccaaactattgtttcccactggatggtgtggctaccaatactgtttaccaaggtgttaagttacaaactggtcaaactgacaaatggcagaaagatgatgatgtttcaacacaaaaccgaattgggaagggtagcgtattcgccatggaaatcaatctccaggccaacctgtggaagagttttctgtactcgaacgtggccttgtacctgcccgactccttcaagtacacgccggccaacgtcacgctgcccaccaacaccaacacctacgagtacatgaacggccgcgtagtagccccctcgctggtggacgcatacgtcaacatcggtgcgcgctggtcgctggaccccatggacaacgtcaaccccttcaaccaccaccgcaacgcgggcctgcgctaccgctccatgcttctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggcaacgacctgcgcgtcgacggcgcctccgtccgcttcgacagcgtcaacctctacgccaccttcttccctatggcgcacaataccgcctccaccctggaagccatgctgcgcaacgacaccaacgaccagtccttcaacgactacctctcagccgccaacatgctctaccccatcccggccaaggccacaaacgtgcccatctccatcccctcgcgcaactgggccgccttccgcggctggagtttcacccgactcaagaccaaggaaactccctccctcggctcgggtttcgacccctactttgtctactcgggctccatcccatacctcgacgggaccttctacctaaaccacaccttcaaaaaggtttccctcatgttcgactcctcggtcagctggcccggcaacgaccggctgctcacgccgaacgagttcgagatcaagcgcagcgtcgacggggagggctacaacgtggcccaatgcaatatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaacaactcgggcttcaccggctaccttgcacccaccatgcgtcaggggcagccctaccccgccaacttcccctacccgctcatcggctccaccgcagtcccctccgtcacccagaaaaagttcctctgcgacagggtcatgtggcgcatccccttctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacacccttctccgccggcaacgccaccacctaa

SEQ ID NO: 43 - DNA, 2811 bp, Adenovirus serotype 24 Hexon gene
atggctaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtgcctcccacccacgatgtgaccacggaccggtcccagcgtctgacgctgcgctttgtgcccgtggatcgcgaggacaccaggtacttgtacaaggcgcgcttcactctggccgtgggcgacaaccgggtgctagacatggccagcacttactttgacatccgcggcgtcctggaccgcggtcccagcttcaaaccctactcgggcacggcttacaacagcctggcccccaaaggcgcccccaactctagtcagtgggaacaagctaaagctaccaatgccggtcaaaaggaaactcacacatttggagtagccgctatgggcggagaagacattacagtgaaaggtcttcaaattggaactgatgaaactaaggaagatggagaggatgaaatttttgcagatcaaacattccagccagaacctcaagtgggagaacagaactggcaagaaacgtttgttttctatggaggcagagctcttaagaaagaaaccaaaatgaagccatgttatggctcttatgcgagacccacaaatgaaaagggaggacaggctaaatttacacttgatgaaaaaggtcagccaaccaaaattcctgatattacaatggatttctttgatagtccacaagatgatacatcaggtgtaactaataagccagatattgtcatgtatgcagaaaatgtaaatttagaagctcctgacacacatgtagtttacaaaccaggcaaagatgattctagttcttccgctaacctcacacaacaggccatgcctaacagaccgaactacatcgggttcagagacaactttgtgggtcttatgtactacaatagtactggcaacatgggtgtgctggctggtcaggcctctcagttgaatgctgtggtcgacttgcaagacagaaacaccgagctgtcttaccagctattgctagattctctgggtgacagaaccagatactttagcatgtggaattctgcagtggacagctatgaccccgatgtcaggatcattgagaatcacggtgtggaagatgaacttccaaactattgcttcccactgaatggcagtggttctaacagcacatacaaaggtgttaaagctggaactggaaacaattgggatgacgatgaaaatgttgcaagacaaaatcagattggcactggcaacctgttcgccatggagatcaacctccaggccaacctatggaagagttttctgtactcgaacgtggccctgtacctgcccgactcctacaagtacacgccggccaacgtcacgctgcccaccaacaccaacacctacgactacatgaacggccgcgtggtagccccctcgctggtggacgcctacatcaacattggcgcccgctggtcgctggaccccatggacaatgtcaatcccttcaaccaccaccgcaacgcgggcctgcgctaccgctccatgctcctgggcaacggccgctacgtgcccttccacatccaagtgccccaaaagttctttgccatcaagaacctgcttctgctccccggttcctacacctacgagtggaacttccgcaaggacgtcaacatgatcctgcagagttccctcggcaacgacctgcgcgtcgacggcgcctccgtccgcttcgacagcgtcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcacccggctcaagaccaaggaaactccctccctcggctcgggtttcgacccctactttgtctactcgggctccatcccctacctcgacgggaccttctacctcaaccacaccttcaagaaggtctccatcatgttcgactcctcggtcagctggcccggcaacgaccggctgctcacgccgaacgagttcgagatcaagcgcagcgttgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctacccttccagcacaacaactcgggcttcaccggctaccttgcgcccaccatgcgccaggggcagccctaccccgccaacttcccctacccgctcatcggctccaccgcagttccctccgtcacccagaaaaagttcctctgcgacagggtcatgtggcgcatcccattctccagcaactttatgtccatgggcgccctcaccgacctgggtcagaacatgctctatgccaactcggcccacgcgctcgacatgacctttgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgctaccacttaa

SEQ ID NO: 44 - DNA, 2841 bp, Adenovirus serotype 25 Hexon gene
atggccaccccatcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacagcttacaacagcctggcccccaagggcgctcccaattccagtcagtgggaagaaaaaaagagtggagcccgaaatcaaaccgaaactcatacgtttggcgttgctcccatgggcggaactaacatcacaattagcggtttgcaaattggaactgaggaagaagatggaaatcctacaaaggagatttttgcagataaaacattccagcctgaacctcaaataggagaagaaaactggcaggacactgagaatttttatggcggtagagctcttaagaaagacaccaaaatgaaaccttgctatggctcttttgccagacctactaacgaaaagggaggtcaagctaagttaaaacttgacgcccaaggtcagtcaactaaagattatgacattgacctggctttctttgactcacctggaggaaatacaacagctggtggtcaagaagagcttaaagcagacattgtcatgtacactgagaatgcttatctggaaacaccagatacccatgtagtttacaagccaggaacctctgatgacagttctgccgccaacttggttcagcaatccatgcccaacagacccaactacattggcttcagagacaactttgtgggtctcatgtattacaacagcactggcaacatgggtgtgctggctggtcaggcctctcagttgaatgccgtggtcgacttacaagacagaaacaccgagctgtcttaccagctcttgctagattctctgggcgacagaaccaggtactttagcatgtggaactctgcggtggacagctatgatcccgacgtcaggatcattgaaaatcacggtgtggaagatgaacttccaaactattgcttcccattggatggctctggcactaattcagcttatcaaggtgttaaagttaaaaatggggaagatggtgatattgagagcgaatgggaaaaagacaccaatgtcgcagctcgtaaccaactgtgcaagggtaacatctttgccatggagatcaacctccaggccaacctgtggaagagttttctgtactcgaacgtggccctgtacctgcccgactcatacaagtacacgccggccaacgtcaagctgcccaccaacaccaacacctacgagtacatgaacgggccgcgtggtagccccctcactggtggacgcctacgtcaacatcggcgcccgctggtcgctggaccccatggacaacgtcaaccccttcaaccaccaccgcaatgcgggcctgcgctaccgctccatgcttctgggcaacggccgctacgtgcccttccacatccaagtgccccaaaagttctttgccatcaagaacctgctcctgctccccggctcctacacctacgagtggaacttccgcaaggacgtcaacatgatcctgcagagttccctcggaaacgacctgcgcgtcgacggcgcctccgtccgcttcgacagcatcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcacccggctcaagaccaaggaaactccctccctcggctcgggtttcgacccctactttgtctactcgggctccatcccctacctcgacgggaccttctacctcaaccacaccttcaagaaggtttccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaacaactcgggcttcaccggctaccttgcacccaccatgcgtcagggggcagccctaccccgccaacttcccctacccgctcatcggctccaaccgcagtcccctccgtcacccagaaaaagttcctctgcgacagggtcatgtggcgcatccccttctccagcaacttcatgtccatgggcgccctcaccgacctgggtcagaacatgctctacgccaactcggcccacgcgctcgacatgactttcgaggtggaccccatggatgagcccaccctcctctatcttctcttcgaagttttcgacgtggtcagagtgcaccagccgcaccgcggcgtcatcgaggccgtctacctgcgcacacccttctccgccggcaacgccaccacctaa

SEQ ID NO: 45 - DNA, 2859 bp, Adenovirus serotype 26 Hexon gene
  modified_base (2151): n = a, c, g or t/u
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggccaccacgtacttgtacaaggcgcgcttcactctggccgtgggcgacaaccgggtgctagacatggccagcacttactttgacatccgcggcgtcctggaccgcggtcccagcttcaaaccctactcgggcacggcctacaacagcctggctcccaagggtgcccccaatcccagtcagtgggaaacaaaagaaaagcaaggaactactggaggagtgcagcaagaaaaagatgtcacaaaaacatttggtgtggctgccaccggcggaattaatataacaaaccagggtctgttactaggaactgacgaaaccgctgagaatggcaaaaaagacatttatgcagacaagactttccagccagaacctcaagttggagaagaaaactggcaggaaaatgaagccttctatggaggaagggctcttaaaaaggacactaaaatgaaaccatgctatggatcttttgctagacctactaatgagaaaggaggtcaggcaaagttcaaaccagttaatgaaggagaacaacctaaagatctggatatagattttgcttactttgacgtccctggcggaagtcctccagcaggtggtagtggggaagaatacaaagcagatataattttgtacactgaaaatgttaatcttgaaacaccagacactcatgtggtttacaagccaggaacttcagataacagttcagaaatcaatctggttcagcagtccatgccaaacagacccaactacattggctttagggacaactttgtaggtctcatgtattacaacagcaccggaaatatgggtgtgctggctggtcaggcttctcagttgaacgctgtggtcgacttgcaagacagaaacaccgagttatcttaccagctattgctagattctctgggtgacagaaccagatactttagcatgtggaactctgcggtggacagttacgatccagatgtcaggatcattgaaaatcacggtgtggaagatgaacttccaaactattgcttcccattgaatggcactggaaccaattccacttatcaaggtgtaaagattacaaatggtaatgatggtgctgaagaaagtgagtgggagaaagacgatgcaatttctagacaaaaccaaatctgcaagggcaatgtctacgccatggagatcaacctgcaggccaacctgtggaagagttttctgtactcgaacgtggccctgtacctgcccgactcctacaagtacacgccggccaacgtcaagctgcccgccaacaccaacacctacgagtacatgaacggccgcgtggtagccccatccctggtggacgcctacatcaacatcggcgcccgctggtcgttggaccccatggacaacgtcaaccccttcaaccacccacgcaatgcgggcctgcgctaccgctccatgctgctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggcaacgacctgcgcgtcgacggcgcctccgtccgcttcgacagcgtcaacctatacgccactttcttccccatggcgcacaacaccgcttcaaccttggaagccatgctgcgcaacgacacccacgaccagtccttcaacgactacctctcggccgccaacatgctctaccccatcccggccaaggccaccaacgtgcccatctccatcccatcgcgcaactgggccgccttccgcggctggagtttcacccggctcaagaccaaggaaactccttccctcggctcgggtttcgacccctactttgtctactcgggctccatcccctacctcgacgggaccttctacctcaaccacacnttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaataactcgggcttcaccggctacctcgcacccaccatgcgccaggggcagccctaccccgccaacttcccctacccgctcatcggtcagacagccgtgccctccgtcacccagaaaaagttcctctgcgacagggtcatgtggcgcatcccattctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtacaccagccgcaccgcggcgtcatcgaggccgtctacctgcgcacgcccttctccgccggcaacgccaccacctaa

SEQ ID NO: 46 - DNA, 2811 bp, Adenovirus serotype 27 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgctttgtgcccgtggatcgcgaggacaccacgtactcatacaaggcgcgcttcactctggccgtgggcgacaaccgggtgctagacatggccagcacgtactttgatatccgcggcgtcctggaccgcggtcccagcttcaaaccctactcgggcacggcttacaacagcctggctcccaagggcgcccccaactccagtcagtggactgctaaagaaaatcaaaaagatgttacaaaaacatttggtgtagctccaatgggaggaattaatatatcaaaggatggtttacaaattggcgttgaagaaacagttgacaagcaagaaaaggaaatttatgctgacaagagtttccagccagaacctccagtaggaaaagaaaactggcaagagtcagaagccttctatggcggcagagctcttaagaaggacaccaagatgaaaccatgctatggttcatttgccagacccactaatgaaaagggaggtcaggcaaaattcaaagcacctgctgtcgaaggagaacagcctaaggaacttgatattgactttgctttctttgacactgactcaggtgacacagaatataaagcagatattgttatgtatgctgaaaacgtgaatctggaaactccagacactcatgttgtttacaaaccaggcaaagaggatgatagctctgaagcaaacttggttcagcagtccatgccaaacagaccaaactacattggctttagagacaattttgtggggctcatgtattacaacagcactggtaacatgggtgtgctggctggtcaggcctctcagttgaatgctgtggtcgacttgcaagacagaaacacagagctgtcttaccagctattgctagattctctgggtgacagaaccaggtactttagcatgtggaactctgcggtggacagctatgatcccgatgtcaggatcattgaaaatcatggtgtggaagatgaacttccaaactattgcttcccattggatggctctggaactaattccacatatcaaggtgtaaaaattacagatggaaatggtgctttgcaaaacggatgggcaaaagatgatgcaatctcgagacaaaaccaaatctgcaagggcaacatctacgccatggagatcaacctccaggccaacctgtggaagagttttctgtactcgaacgtggccctgtacctgcccgactctttcaagtacacgccggccaacgtcactctgcccaccaacaccaacacctacgagtacatgaacggccgcgtggtggccccctcgctggtggacgcctacatcaacatcggcgcccgctgggcgctggaccccatggacaatgtcaaccccttcaaccaccaccgcaacgcgggcctgcgctaccgctccatgttgctaggcaacggccgctacgtgcccttccatatccaagtgccccaaaagttctttgccatcaagaacctgctcctactcccgggctcctacacctacgagtggaacttccgcaaggacgtcaacatgatcctgcagagttccctcggcaacgacctgcgcgtcgacggcgcctccgtgcgcttcgacagcgtcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcactcgcctgaaaaccaaggaaactccctcgctcggctcgggtttcgacccctactttgtctactcgggctccattccctacctcgacgggaccttctacctaaaccacaccttcaagaaggtctccatcatgttcgactcctcggtcagctggcccggcaacgaccggctgctcacgccgaacgagttcgaaatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtattccttcttccgcaacttccagcccatgagcaggcaggtggtcgatgagatcaactacaaggactacaaggccgtcaccctgcccttccagcacaacaactcgggcttcaccggctacctcgcacccaccatgcgtcaggggcagccctatcccgccaacttcccctacccgctcatcggctccaccgcagtgccctccgtcacccagaaaaagttcctctgcgacagggtcatgtggcgcatccccttctccagcaacttcatgtccatgggcgcccttaccgacctgggtcagaacatgctctacgccaactcggctcacgcgctcgacatgaccttcgaggtggaccccatggatgagcccaccctcctctatcttctctttgaagttttcgacgtggtcagagtgcaccagccgcaccgcggcgtcatcgaggccgtctacctgcgcacgcccttctccgccggcaacgccaccacctaa

SEQ ID NO: 47 - DNA, 2832 bp, Adenovirus serotype 28 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggccccagcttcaaaccctactcgggcacggcttacaacagcctggctcccaagggcgcccccaactccagtcagtgggatgcgcaagaaaaaagtggacaaggaagtgacatggttactaaaactcacacatttggcgtggcagccatgggaggagaaaacattacaaaaaatggtcttcaaattggcactgaaattaccgccgacaatcaaaagaaagaaatttttgcaaacaaaacataccagccagaacctcaagtaggagaggagaactggcaagaaaatgaagtcttctatggcggcagagctctcaagaaagaaacaaaaatgaagccttgttatggttcatttgccagacctaccaatgaaaatggaggtcaggcaaaattcaaaacacctgctgaaggacaggaacctaaagaacttgacattgaccttgctttctttgacactgacggcggaacagctgacactgaatataaagcagatattgttatgtatgctgaaaatgtgaatcttgaaactccagatacacatgtagtttacaaaccaggcaaggaagatgacagttcagaaatcaatctagttcagcagtccatgcccaacagaccaaactacattggtttcagggacaactttgtaggtctcatgtattacaatagcactggcaacatgggtgtgctggctggtcaggcttctcagttgaacgctgtggtcgacttgcaagacagaaacaccgaactgtcttaccagctcttgctagattctctgggtgacagaaccagatactttagcatgtggaactctgcggtggacagttacgatccagatgtcaggatcattgaaaatcacggtgtggaagatgaacttccaaattattgcttccctcttgacggattgggaactaatgctacctatcaaggtgtaaaagtatcaactggagatggtgctacgcaaagcggatgggcaaaagacgacacaatggccaggcaaaaccaaatatgcaggggtaacatctacgccatggagatcaacctccaggccaacctctggaagagttttctgtactcgaacgtagccctgtacctgcctgactcatacaagtacacgccggccaacgtcacgctgcccgccaataccaacacctacgagtacatgaacggccgcgtggtagccccctcgctggtggacgcctacatcaacatcggcgcccggtggtcgctggaccccatggacaacgtcaacccattcaaccaccaccgcaacgcaggcctgcgctaccgctccatgcttctgggcaacggccgctacgtgcccttccacatccaagtgccccaaaagttctttgctatcaagaacctgctcctgctccccggctcctacacctacgagtggaacttccgcaaggatgtcaacatgatcctgcagagttccctcggaaacgatctgcgcgtcgacggcgcctccgtccgcttcgacagcgtcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcacgcaactgggccgccttccgcggctggagtttcaccaggctcaagaccaaggaaactccctcgctaggctcgggtttcgacccatactttgtctactcgggctccatcccctatctcgacgggaccttctacctcaatcacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaacaactcgggcttcaccggctacctcgcacccaccatgcgtcaggggcagccctaccccgccaacttcccctacccgctcatcggccagacagccgtgccctccgtcacccagaaaaagttcctctgcgacagggtcatgtggcgcatccccttctccagcaacttcatgtccatgggcgccctcaccgacctgggtcagaacatgctctacgccaactcggcccatgcgctcgacatgaccttcgaggtggaccccatggatgagcccaccctcctctatcttctcttcgaagttttcgacgtggtcagagtgcaccagccgcaccgcggcgtcatcgaggccgtctacctgcggacgcccttctccgccggaaacgccaccacataa

SEQ ID NO: 48 - DNA, 2826 bp, Adenovirus serotype 29 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcctacaacagtctggcccccaagggcgcccccaactccagtcagtgggaacagaaaaaggccaatgctggagatcaaaaggaaacacatacttatggtgtagctcctatgggtggagaaaacattacaattagcggtttgcaaattggaacagatactacaaatggcaaacaagacccgatatatgctaataagctgtatcaaccagagcctcaagtaggagaagaaaactggcaggaaacagaagccttctatggaggaagggctcttaaaaaggaaaccaagatgaaaccatgctatggctcatttgccagacccacaaatgaaaaaggaggacaggcaaaactaagagaccctgaaaaaagtcaagaagattttgacatagcactagcattctttgatactccgggaggaactttaacaggtggtggaacggaatacaaagcagacattgttatgtgcactgaaaatgttaatcttgaaaccccggacacccacgtggtgtataaaccaggcaaagatgatgacagttcagaaatcaacttgggtcagcagtccatgcccaacagacctaactacatcggcttcagggacaactttgtgggtctcatgtactacaacagcactggcaacatgggtgtgctggccggtcaggcttctcagttgaatgctgtggtcgacttgcaagacagaaacacagagctgtcttaccagctcttgctagattctctgggcgacagaaccaggtactttagcatgtggaactctgcggtggacagctatgatcccgatgtcaggatcattgagaatcacggtgtggaagatgaacttcccaactattgcttcccattggatgggtctggcaccaatgctgcttatgaaggtgtaaaagttaaaaatggacaagatggggatcaagagagcgaatgggaaaaagacaccaatgtggcagatcgaaaccaaatatgcaagggcaacatctacgccatggagatcaacctccaggccaacctgtggaagagttttctgtactcgaacgtggcgctgtacctgcccgactcctacaagtacacgccggccaacgtcacgctgcccaccaacaccaacacctacgagtacatgaacggccgcgtggtggccccctcgctggtggacgcctacatcaacatcggcgcgcgctggtccctggaccccatggacaacgtcaaccccttcaaccaccaccgcaacgcgggtctgcgctaccgttccatgctgctgggcaacggccgctacgtgcccttccacatccaagtgccccaaaagttctttgccatcaagaacctgctcctgctcccgggctcctacacctacgagtggaacttccgcaaggatgtcaacatgatcctgcagagttcccttggcaacgacctgcgcgtcgacggcgcctccgtccgcttcgacagcgtcaacctctacgccaccttcttgcccatggcacacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgcctttcgcggctggagtttcactcgcctgaaaaccaaggaaactccttccctcggctcgggtttcgacccctactttgtctactcgggctccatcccctacctcgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttttacgtgcccgagggttacaaggaccgcatgtactccttcttccgcaacttccagcccatgagcaggcaggtggtcgatgagatcaactacaaggactacaaggcagtcaccctgcccttccagcacaacaactcgggcttcaccggctacctcgcacccaccatgcgtcagggacagccctaccccgccaacttcccctacccgctcattggctccaccgcagtcccctccgtcacccagaaaaagttcctctgcgacagggtcatgtggcgcatccccttctccagcaacttcatgtccatgggcgcccttaccgacctgggtcagaacatgctctacgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacctaa

SEQ ID NO: 49 - DNA, 2826 bp, Adenovirus serotype 30 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcctacaacagtctggcccccaagggcgcccccaactccagtcagtgggaacagaaaaaggccaatgctggagatcaaaaggaaacacatacttatggtgtagctcctatgggtggagaaaacattacaattagcggtttgcaaattggaacagatactacaaatggcaaacaagacccgatatatgctaataagctgtatcaaccagagcctcaagtaggagaagaaaactggcaggaaacagaagccttctatggaggaagggctcttaaaaaggaaaccaagatgaaaccatgctatggctcatttgccagacccacaaatgaaaaaggaggacaggcaaaactaagagaccctgaaaaaagtcaagaagattttgacatagacctagcattctttgatactccgggaggaactttaacaggtggtggaacggaatacaaagcagacattgttatgtgcactgaaaatgttaatcttgaaaccccggacacccacgtggtgtataaaccaggcaaagatgatgacagttcagaaatcaacttggttcagcagtccatgcccaacagacctaactacatcggcttcagggacaactttgtgggtctcatgtactacaacagcactggcaacatgggtgtgctggccggtcaggcttctcagttgaatgctgtggtcgacttgcaagacagaaacacagagctgtcttaccagctcttgctagattctctgggcgacagaaccaggtactttagcatgtggaactctgcggtggacagctatgatcccgatgtcaggatcattgagaatcacggtgtggaagatgaacttcccaactattgcttcccattggatgggtctggcaccaatgctgcttatgaaggtgtaaaagttaaaactggtcaagatggggatcaagagacggaatgggaaaaagacaccaatgtggcagatcgaaaccaaatatgcaagggcaacatctacgccatggagatcaacctccaggccaacctgtggaagagttttctgtactcgaacgtggcgctgtacctgcccgactcctacaagtacacgccggccaacgtcacgctgcccaccaacaccaacacctacgagtacatgaacggccgcgtggtggccccctcgctggtggacgcctacatcaacatcggcgcgcgctggtcgctggaccccatggacaacgtcaaccccttcaaccaccaccgcaacgcgggtctgcgctaccgttccatgctgctgggcaacggccgctacgtgcccttccacatccaagtgccccaaaagttctttgccatcaagaacctgctcctgctcccgggctcctacacctacgagtggaacttccgcaaggatgtcaacatgatcctgcagagttcccttggcaacgacctgcgcgtcgacggcgcctccgtccgcttcgacagcgtcaacctctacgccaccttcttccccatggcacacaacaccgcctccaccctggaagccatgctgcgcaacgacaccaacgaccagtccttcaacgactacctctcagccgccaacatgctctaccccatcccggccaaggccaccaacgtgcccatctccatcccctcgcgcaactgggccgcctttcgcggctggagtttcactcgcctgaaaaccaaggaaactccttccctcggctcgggtttcgacccctactttgtctactcgggctcgatcccctaccttgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggatacaaggaccgcatgtactccttcttccgcaacttccagcccatgagcaggcaggtggtcgatgagatcaactacaaggactacaaggccgtcaccctgcccttccagcacaacaactcgggcttcgtcggctacctcgcacccaccatgcgccaggggcagccttaccccgccaacttcccctacccgctcatcggccagacagccgtgccctccgtcacccagaaaaagttcctctgcgacagggtcatgtggcgcatccccttctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacctaa

SEQ ID NO: 50 - DNA, 2769 bp, Adenovirus serotype 31
atggccactccctcgatgatgccgcagtggtcttacatgcacatcgccggtcaggacgcctcggagtacctgagtcccggtctggtgcaattcgcccgcgccacggacacctacttcaccctgggaaacaagtttaggaatcccactgtggctcccacccatgatgtcaccaccgaccgctcgcagcggttgacgctgcgttttgtgcccgtggatcgggaagatactgcttattcttacaaggctcgctttacgctggctgtgggggacaatcgcgtgcttgacatggctagctcttactttgatattagaggggtgctggatcgcggtcccagttttaagccttattcgggtaccgcctataactctttagctccaaagggcgcgcccaatgccagccaatggctaactacaaacaatggcaacaaaactcacacttttgctcaagcgccttacataggagactctatcagtaaagacggcattcaagttgggacaaacactgccaatccccagcaagcagtatatgccgacaaaacataccagccagaacctcaagtaggcgaaagtcaatggaacgcttcagtatctaacaatgcaaaagcagcaggaagggtgttaaaaagcactacgcccatgtatccgtgctatgggtcttatgcacgagcaacaaatgaaaaaggaggtcaatcaaaaaataacgacggtgtggaaatgcgattttttgcatccagtgcgaatgacaatcaaaatgatccaactgttgtaatgtatagcgaggacgtaaacttagaggctcctgacacacacattgtttacaaaccagctgtaactgcggacactatcagttcagaactcctcctaggacaacaagcagcacctaacagaccaaattacattgccttcagagacaattttattgggcttatgtattacaacagcactggaaacatgggagtacttgcaggacaggcttctcagctcaatgcagtggttgatctgcaagataggaacacagagctgtcctaccaacttatgttggatgctttaggcgatagaagccgttatttttccatgtggaattctgccgtcgacagctacgatccagacgtgcgaattattgaaaatcatggtgtggaagacgaactgcccaattactgttttccgttaggggctgtaggcgttgtgaattcatacaaaggtgtgaaacaaaatgacggaaatggttgggtaaaagatgacagtgtagcagataccaatgacatagccaaaggtaatatatttgccatggaaattaacttacaggcaaatttgtggagaagttttctgtattcaaatgtggggctgtacctgcctgacgacttgaaatacacccccgggaacataaaacttccagaaaacaaaaacacctacgagtacatgaacgggcgcgtgactgttcccggtctggtagacacctacgttaacattggcgcgcgctggtctcctgacgtaatggataacataaacccttttaaccaccaccgaaacgcagggttgcgctataggtccatgttactgggcaacggtagatttgttccgtttcacattcaggtacctcagaaatttttcgctattaagaatttgctgctgttaccagggtcctacacttacgagtggaacttcagaaaagatgtaaacatgatacttcagagcaccctgggcaatgatcttagagtggacggagccagcattcgctttgacaacattgccctgtatgcaaacttcttccccatggctcacaatacagcttctacattggaagccatgctgcgaaatgacaccaatgaccagtctttcaacgattacttgtgcgcggcgaacatgttgtatcccattccagcaaatgccaccagcgtgcccatttcaataccttcgcgaaattgggcagccttcagagggtggagttttactcgccttaaaactaaagagacaccttctcttggttcagggtttgacccctactttgtttactctggaaccattccctacctggacgggactttttacctgaaccacacttttaaaaaggtgtcaatcatgtttgactcctctgttagctggcctggaaatgacaggctgttaacaccaaatgaatttgaaataaaacgttctgtagacggggaaggctacaatgtggcccaatgtaatatgaccaaagattggtttctagtacaaatgctcagtcattataatattggataccagggtttttacgttccagagagctacaaagaccgcatgtattctttcttcagaaactttcagcccatgagcaggcaggttgtagacactactgaatacaaggagtataaaaaagtaaccgtggaattccagcataacaactcagggtttgtgggatacctgggtcctactatgcgcgagggacaagcttatcccgcaaactacccctatcctctcataggaaaaaccgctgtacaaagcgtcacacaaaaaaagtttctctgtgaccgtgtgatgtggcgcattccattttctagcaattttatgtcaatgggggccgttacagacctcgggcaaaatatgctgtatgcaaattcggcccatgcgttagatatgacatttgaagttgaccccatggaagagcccacccttctttatgttttgtttgaagttttcgacgtggtgcgcattcatcaaccacaccgcggagtcattgaagcggtctatctgcgcacacccttctccgcgggtaacgccaccacctaa

SEQ ID NO: 51 - DNA, 2838 bp, Adenovirus serotype 32 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacgtactttgacatccgcggcgtcctggaccgcggtcccagcttcaaaccctattcgggcacagcttacaacagcctagcccccaagggcgctccaaactccagtcagtgggctgcaaaagacgccaacactgctgatcaaacagttaaaacacacacacatggcgtagctgctatgggaggaacagatattacagcaaagggtttgcaaattggagttgatactactgaaaataatgcaggtcctatttatgcaaatgaaatataccagccagagcctcaaataggagaagaaaacttgcaagatgttgaaaattactatggaggcagagctcttaagaaagaaacaaaaatgaaaccttgctatggctcatttgccagacccacaaatgaaaaaggcggtcaagccaaatttataactggagaagatggtcagccaactaaaaatcatgatatcacaatgaatttctttgacactcctggtggcactattggtcaagctgatgaacttgaagcagacattgttatgtatgctgagaatgtgcatctggaaactccagacacgcatgtggtgtacaaaccaggaacttctgatgaaagttccgaagcaaatttggttcagcagtccatgccaaacaggcccaactatattggcttcagggacaactttgtgggtcttatgtattacaacagtacaggcaacatgggtgtgctggcaggtcaggcttctcagttgaatgctgtggtcgacttgcaagacagaaacacagagctgtcttaccagctcttgctagattctctgggtgacagaaccagatactttagcatgtggaactctgcggtggacagctatgatcccgatgtcagaatcattgaaaatcacggtgtggaagatgaacttccaaactattgttttccactggacggagctggcactaatgctacatatcaaggtgtaaaagttaaagatggcgaagatggaaatgagaatgctgattgggtaaaagatccaaatcttgcttctcgaaaccaaatatgcaagggtaacatcttcgccatggagatcaacctccaggccaacctgtggaagagttttctgtactcgaacgtggcgctgtatctgccagactcctacaagtacacgccggccaacatcacgttgcccaccaacaccaacacctacgagtacatgaacggccgcgtggtggccccctcgctggtggacgcctacatcaacatcggcgcccgctggtcgctggaccccatggacaacgtcaaccccttcaaccaccaccgcaatgcgggcctgcgctaccgctccatgcttctgggcaacggccgctacgtgcccttccacatccaagtgccccaaaagttctttgccatcaagaacctgctcctgctcccgggctcctacacctacgagtggaacttccgcaaggatgtcaacatgatactgcagagttccctcggcaacgacctgcgcgtcgacggcgcctccgtccgcttcgacagcgtcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcacccggctcaagaccaaggaaactccctccctcggctcgggtttcgacccctactttgtctactcgggctccatcccctacctcgacgggaccttctacctcaaccacaccttcaagaaggtctccatcatgttcgactcctcggtcagctggcccggcaacgaccggctgctcacgccgaacgagtttgagatcaagcgcagcgtcgacggggagggctacaacgtggcccaatgcaatatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaacaactcgggcttcaccggctaccttgcacccaccatgcgtcaggggcagccctaccccgccaacttcccctacccgctcatcggctccaccgcagtcccctccgtcacccagaaaaagttcctctgcgacagggtcatgtggcgcatccccttctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccaccaaagcatga

SEQ ID NO: 52 - DNA, 2832 bp, Adenovirus serotype 33 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctcgaccgcggtcccagcttcaaaccctactcgggcacggcttacaacagcctggcccccaagggcgcccccaattccagtcagtgggaacagaaaaaggccactgggggagctgatgcaaaggaaacacatacattcggtgtagccgctatgggaggattgaacattacagacaaaggtttgcaaattggaatagatgaagataatgtagatggagatgatgagatatatgctgacaaaacttttcaaccagaacctcaagtgggagaggaaaactggaaagaaagttttaatttctatggaggcagagctattaagaaagacaccaaaatgaagccatgctatggttcctttgccagacccaccaatgttaagggaggtcaggctaaacttaaaactggagaagatatagaccctaacaaggactttgacatagacatggctttctttgatctaaagcaagctgacacaggaggtaataacaatcaagcagacgtggttatgtataccgaaaatattcatctggaaactccagacactcatgtggtgtacaagccaggcaaggaagatgcaagttctgaaattaacttgactcagcagtccatggccaacagacccaactacattggcttcagagacaactttgtagggctcatgtactacaacagcactggcaacatgggtgtgctggccggtcaggcatctcagttgaatgcagtggtcgacttgcaagacagaaacaccgagctgtcttaccagctcttgctagattctctgggcgacagaaccagatactttagcatgtggaactctgctgtggacagctatgatcccgatgtcaggatcattgagaatcacggtgtggaagatgaacttcccaactattgcttcccattggacggatctggcagcagtactgcttttcagggagttaaagtaaaaaccccggctggtactggtcaaaacactgtatgggaagttaacaatgatgctgcaacgcataaccaaattgcaagaggcaacctgtacgccatggagatcaacctccaggccaacctgtggaagagttttctgtactcgaacgtggccctgtacctgcccgactcctacaagtacactccggccaatgtcacgctgcccaccaacaccaacacctacgagtacatgaacggccgcgtggtggccccctcgctggtggacgcctacatcaacatcggcgcccgctggtcgctggaccccatggacaacgtcaaccccttcaaccaccaccgcaacgcgggcctgcgctaccgctccatgcttctgggcaacggccgctacgtgcccttccacatccaagtgccccaaaagttctttgccatcaagaacctgctcctgctcccgggctcctacacctacgagtggaacttccgcaaggatgtcaacatgatcctgcagagttccctcggcaacgacctgcgcgtcgacggcgcctccgtccgcttcgacagcgtcaacctctacgccaccttcttccccatggcacacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcacccggctcaagaccaaggaaactccctccctcggctcgggtttcgacccctactttgtatactcgggctccatcccctaccttgacgggaccttctacctcaaccacaccttcaagaaggtctccatcatgttcgactcctcggtcagctggccaggcaacgaccggctgctcacgccgaacgagttcgagatcaagcgcagcgtcgacggggagggctacaacgtggcccaatgcaacatgaccaaggactggttcctcgtccagatgctctcccactacaacatcggttaccagggcttccatgtgcccgagggctacaaggaccgcatgtactccttcttccgcaacttccagcccatgagcaggcaggtggtcgatgagatcaactacaaggactacaaggccgtcaccctgcccttccagcacaacaactcgggcttcaccggctacctcgcgcccaccatgcgccaggggcagccctaccccgccaacttcccctacccgctcatcggctccaccgccgtgccctccgtcacccagaaaaagttcctctgcgacagggtcatgtggcgcatccccttctccagcaacttcatgtccatgggcgccctcaccgacctgggtcagaacatgctctatgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacataa

SEQ ID NO: 53 - DNA, 2835 bp, Adenovirus serotype 36 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccatgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacgtactttgacatccgcggcgtcctggaccgcggtcccagcttcaaaccctactcgggcacggcttacaacagtttggcccccaagggcgcccccaactccagtcagtggactgacaaagaacggcaaaatggtggacaaccacccactacaaaagatgttacaaaaacattcggagtagcagccaggggagggcttcatattactgataaaggactacaaataggagaagatgaaaataacgaggatggtgaagaagagatatatgcagacaaaactttccagccagaacctcaagtaggagaggaaaactggcaagatactgatgttttctatggcggcagagcgcttaaaaaggaaaccaaaatgaaaccatgctatggctcttttgccagacctaccaatgaaaaaggaggtcaagctaaatttttaaatggcgaaaacggtcaaccttctaaagatcaagatattacattagctttctttgatcttaaacaaaatgacactggaactactcaaaaccagccagatgttgtcatgtacactgaaaatgtgtatctggaaaccccagacacccatgtggtgtacaaacctggcaaggaagatacaagctccgctgctaaccttacacaacagtccatgcccaacaggcccaactacattggtttcagggacaactttgtggggctcatgtattacaacagcactggcaacatgggtgtgctggctggtcaggcctctcagttgaatgctgtggttgacttgcaagacagaaacaccgagctgtcatatcagctcttgctagattctctgggtgacagaaccagatactttagcatgtggaattctacggtggacagctatgatccagatgtcaggatcattgagaatcacggtgttgaagatgagcttccaaattattgcttcccactggatggatctggcagcaataccgcatatcaaggtgttaaatatgaaaacggagctggcaatggaagctggaaagtagatggcgaagttgcttctcagaatcagatcgccaagggtaatctgtatgccatggagataaaccttcaggccaacctgtggaagagttttctgtactcgaacgtggcgctgtatctaccagactcctacaagtacacgccggccaacatcacgctgcccaccaacaccaacacctacgagtacatgaacggccgcgtggtggcaccctcgctggtggatgcctatgtcaacatcggtgcccgctggtcgctggaccccatggacaacgtcaaccccttcaaccaccaccgcaacgcgggtctgcgctaccgctccatgcttctgggcaacggccgctacgtgcccttccacatccaagtgccccaaaagttctttgccatcaagaacctgctcctgcttcccggttcctacacctacgagtggaacttccgcaaggatgtcaacatgatcctgcaaagttccctcggcaacgacctgcgcgtcgacggcgcctccgtccgcttcgacagcgtcaacctctatgccaccttattccccatggcgcgcaacaccgcctccacccttgaagccatgctgcgcaacgacaccaacgaccagtccttcaacgactacctctcggccgccaacatgctctacccaatcccggccaaggccaccaacgtgcccatctccatcccctcgcgcaactgggccgccttccgcggctggagtttcacccggctcaagaccaaggaaactccctccctcggctcgggtttcgacccctactttgtctactcgggctccattccctacctcgacggaaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgactaaggactggttcctcgtccagatgctctctcattacaacattggctaccagggcttctacgtgcctgagggttacaaggaccgcatgtactccttcttccgcaacttccagcccatgagcaggcaggtggtcgatgagatcaactacaaggactacaaggccgtcaccctgcccttccagcacaacaactcgggcttcaccggctacctcgcacccaccatgcgtcaggggcagccataccccgccaacttcccctacccgctcatcggccagacagccgtgccctccgtcacccagaaaaagttcctctgcgacagggtcatgtggcgcatccccttctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggaaacgccaccacataa

SEQ ID NO: 54 - DNA, 2820 bp, Adenovirus serotype 37 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgctttgtgcccgtggatcgcgaggacaccacgtactcgtacaaggcgcgcttcactctggccgtgggcgacaaccgggtgctagacatggccagcacttactttgacatccgcggcgtcctggaccgcggcccaagcttcaaaccctactcgggcacggcttacaacagcctggcccccaagggcgcccccaatcccagtcagtggactaccaaagaaaagcaaaacggaggaactggagcagaaaaagatgttacaaagacatttggacttgccgccatgggaggcagtaatatttctaaagacggtttgcagattggaactgacaaaacagcaaatgctgaaaaaccaatctatgcagacaaaactttccagccagaacctcaagttggagaagaaaactggcaggataatgatgaatattatggcggcagggctcttaaaaaagataccaaaatgaagccatgctatggttcatttgctaaacccacaaacaaggaaggtgggcaggctaaattgaaagaaacacccaatggtaccgatcctcaatacgatgtggacatggctttctttgactcaagcactataaatataccagatgtggtgttgtacactgaaaatgtagatttggaaactccagatacacatgtggtgtacaaaccaggcaaagaggatgacagttctgaagctaatttaactcagcagtccatgcctaacagaccaaactacattggcttcagagacaactttgtggggctattgtactacaacagcactggcaacatgggtgtgctggctggtcaggcttctcagttgaatgccgtggtcgacttgcaagacagaaacaccgaactgtcttaccagctcttgctagattctcttggtgacagaaccagatattttagtatgtggaactctgcggtggacagctatgatcccgatgtcaggatcattgagaaccacggtgtggaagatgaacttcctaactattgcttccccttggacggtgttcaaactaattcagcctatcaaggtgttaaactaaagcctgatcaaacaggaggcggagttaatggagattgggtaaaggatgatgacatttcagcccataatcaaattggaaagggcaacatctttgccatggagatcaacctccaggccaacctgtggaagagttttctgtactcgaacgtggccctgtacctgcccgactcctacaagtacacgccggccaacgtcacgctgcccgccaacaccaacacctatgagtacatgaacggccgcgtggtagccccctcgctggtggacgcctacattaacatcggcgcccgctggtcgctggaccccatggacaacgtcaacccctttaaccaccaccgcaatgcgggcctgcgctaccgctccatgcttttgggcaatggccgctacgtgcccttccacatccaagtgccccaaaagttctttgccatcaagaacctgctcctgctccccggctcctacacctacgagtggaacttccgcaaggatgtcaacatgatcctgcagagttccctcggaaacgacctgcgcgtcgacggcgcctccgtccgcttcgacagcgtcaacctctacgccaccttctttcccatggcgcacaacaccgcctccaccctggaagccatgctgcgcaacgacaccaacgaccagtccttcaacgactacctctcggccgccaacatgctctaccccatcccggccaaggccaccaacgtgcccatttccatcccctcgcgcaactgggccgccttccgcggctggagtttcacccggctcaagaccaaggaaactccctcccttggctcgggttttgacccctactttgtctactcgggctccatcccctacctcgacgggaccttctacctcaaccacaccttcaagaaggtttccatcatgttcgactcctcggtcagctggcccggcaacgaccggctgcttacgccgaacgagttcgagatcaagcgcagcgtcgacggggagggctacaacgtggcccaatgcaacatgaccaaggactggttcctcgtccagatgctctcccactacaacatcggctaccagggcttccatgtgcccgagggctacaaggaccgcatgtactccttcttccgcaacttccagcccatgagcaggcaggtggtcgatgagatcaactacaaggactacaaggcagtcaccctgcccttccagcacaacaactctggcttcaccggctacctggcacccaccatgcgtcaggggcagccctaccccgccaacttcccctacccgctcatcggctccaccgcagtgccatccgtcacccagaaaaagttcctctgcgacagggtcatgtggcgcatccccttctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacctaa

SEQ ID NO: 55 - DNA, 2823 bp, Adenovirus serotype 38 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggccccagcttcaaaccctactcgggcacggcttacaacagcctggctcccaagggcgcccccaactccagtcagtgggatgctaaagaaaaaaatggtggagctgaaactaccaaaacttatacatacggtgtggctgccatgggaggacttgacataacagacaaaggtcttcaaattggaactgatgaaaccaaagaagataataatgaaatttttgcagacaaaactttccaaccagaacctcaagttggagaggagacatggcaggaaaatgaagttttctatggaggcagggctctcaaaaaagaaaccaaaatgaaaccatgctatggctcttttgctagaccgactaatgaaaaaggaggtcaggctaagtttttaaatgatgatcaaggtctaccttctaaagaacaagacataacaatggctttctttgatagtccacaagctgacacctcaggtgttgataacaaaccagatatggtaatgtacaccgaaaatgtttatctggaaacacctgacactcatgtggtgttcaagccaggaaaagaggatgacagttcagaagtaaatttaactcagcagtccatgccaaacaggccaaattacatcggtttcagagacaattttgtgggcctcatgtattacaacagcactggtaacatgggtgtgctggcgggccaggcttctcagttgaatgctgtggtcgacttgcaagacagaaacaccgaactgtcttaccagctcttgctagattctctgggtgacagaaccagatactttagcatgtggaactctgcggtggacagctatgatcccgatgtcaggatcattgagaatcatggtgtggaagatgaacttcccaactattgcttcccattggatggctctggtagcagttcaacatatcaaggtgttaaatatgaaaacggagctggtggaaatggaacttggaaagtagatgacacagttacaaggcaaaatcagattgccaagggcaacctgtatgccatggagatcaacctccaggccaacctgtggaagagttttctgtactcgaacgtggccctgtacctgcccgactcctacaagtacacgccggccaacgtcacgctgcccaccaacaccaacacctacgactacatcaacggccgcgtagtagcaccctcgctggttgacgcctacatcaacatcggcgcccgctggtcgctggaccccatgaacaacgtcaaccccttcaaccaccaccgcaacgcgggcctgcgctaccgctccatgcttctgggcaacggccgctacgtacccttccacattcaagtgccccaaaagttctttgccatcaagaacctgctcctgctcccgggctcctacacctacgagtggaacttccgcaaggacgtcaacatgatcctgcagagttccctcggaaacgacctgcgcgtcgacggcgcctccgtccgcttcgacagcgtcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcacccggctcaagaccaaggaaactccctccctcggctcgggtttcgacccctactttgtctactcgggctccatcccctacctcgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccattacaacatcggctaccagggcttccacgtgcccgagggctacaaggaccgcatgtactccttcttccgcaacttccagcccatgagcaggcaggtagtcgatgagatcaactacaaggactacaaggccgtcaccctgcccttccagcacaacaactcgggcttcactggctacctcgcacccaccatgcgtcaggggcagccataccccgccaacttcccctacccgctcatcggccagacagccgtgccctccgtcacccagaaaaagttcctctgcgacagggtcatgtggcgcatccccttctccagcaacttcatgtccatgggcgcccttaccgacctgggtcagaacatgctctatgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacctaa

SEQ ID NO: 56 - DNA, 2820 bp, Adenovirus serotype 39 Hexon gene
atggccaccccctcgatgatgccgcagtgggcgtacatgcacatcgccgggcaggacgcctcggagtacctgagcccgggtctggtgcagtttgcccgcgccaccgactcgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacgtactttgacatccgcggcgtcctggaccgcggccccagcttcaaaccctactcgggcacggcctacaacagtctggctcccaagggcgcccccaatcccagtcagtggattgcaaaagaaaagcaaaatggaccacctcagacagaaaaaaatgttacaaaaacatttggagtggctgccatgggaggacttgatataactaatgaaggattgcaaataggagttgaagaaattaatgatgtagaagaagaagtttttgctgacaaaacattccagcctgagcctcaggtcggagaggaaaactggcatgaaacttttaatttctatggaggcaggactctcaaaaaagaaactaaaatgaagccctgctatggatcatttgcaaggcctacaaatattaagggaggacaagctaaacttaaaactggagagaatgtcgatccaacgaaggatttcgacattgatatggctttcttcgatcttaagcaaacagacactggaacaacgcaaaatcagccagatatagttatgtacactgaaaatgtaaaccttgaaactccagacacacatgtggtgtacaagccaggcaaggaagatgcaagttctgaaattaacttgactcaacagtccatggccaacagacccaactacatcggcttcagggacaactttgtggggctaatgtattacaacagtactggaaacatgggtgtgctggctggtcaggcttcccaattgaatgctgtggtcgacttgcaagatagaaacaccgagctgtcttaccagctattgctagattctctgggtgacagaaccagatactttagcatgtggaactctgcggtggacagctatgatcccgatgtcaggatcattgaaaatcacggtgtggaagatgaacttccaaactattgctttccacttgatggcatgggttcaaatgcggcctaccaaggtgttaaaccaaaaactggcaacggatgggatccagatacagatgttgctgcccagaatcaaatcgccaagggcaacatctatgccatggagatcaacctccaggccaacctgtggaagagttttctatattcgaacgtggccttgtacctgcccgactctttcaagtacaccccggccaacgtcactctgcccaccaacaccaacacctacgagtacatgaacggccgcgtggtggccccctcgctggtggacgcctacatcaacatcggcgcccggtggtcgctggaccccatggacaacgtcaaccccttcaaccaccaccgcaacgcgggcctgcgctaccgctccatgctgctgggcaacggccgctacgtgcccttccacatacaagtgccccaaaagttctttgccatcaagaacctgctcctgctcccgggctcctacacctacgagtggaacttccgcaaggacgtcaacatgatcctgcagagttccctcggcaacgacctgcgcgtcgacggcgcctccgtccgcttcgacagcgtcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcactcgcctgaaaaccaaggaaactccctccctgggctcgggtttcgacccctactttgtctactcgggctcgatcccctatctcgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggctgtcaccctgcccttccagcacaacaactcgggcttcaccggctaccttgcgcccaccatgcgccaggggcagccctaccccgccaacttcccctacccactcatcggctccaccgcagtcccctccgtcacccagaaaaagttcctctgcgatagggtcatgtggcgcatccccttctccagcaacttcatgtccatgggcgccctcaccgacctgggtcagaacatgctctatgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacacccttctccgccggcaacgccaccacataa

SEQ ID NO: 57 - DNA, 2847 bp, Adenovirus serotype 42 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcctacaacagcctggcccccaagggcgcccccaactccagccagtgggctgacaaagaaagggtgaatggaggcggtaacaccaaagatgttacaaagacatttggtgtggcagccatgggtggagaagacattacggaaaagggtctgaagattggaactgatccaacagcaaatgaaccaatatttgcagacaaaaattttcagcctgaaccccaagtgggagaggaaaactggcaggaaacatttgttttctatggaggaagagctcttaaaaaagaaaccaaaatgaagccttgctatggttcatttgccagaccgactaatgaaaaaggtggccaggctaaatttataattggagataatggtcaacccactgaaaaccatgacataacaatggctttctttgacactcctggtggtacgatcacaggaggtactggtggtccccaagatgaacttaaagctgacattgttatgtacactgaaaatataaatcttgaaactccagatacccatgtggtgtacaagccaggcaaagaagatgatagctcagaaattaacttggtgcaacagtccatgcccaacagacccaactacatcggtttcagagacaactttgtgggacttatgtactacaacagcactggcaacatgggcgtgctggctggtcaggcctctcagttgaatgctgtggtcgacttgcaagacagaaacaccgagctgtcttaccagctcctgctagattctctgggtgacaggaccagatactttagcatgtggaactctgcggtggacagttatgatcccgatgttaggatcattgagaatcacggtgtggaagatgaacttcccaactattgttttccattggacggctcaggcactaattcagctttccaaggtgtaaaaattaagcaaaaccaagacggagatgttaatgatgattgggaaaaggatgataaggtgtccactcaaaaccaaatttgcaagggcaacatttacgccatggagatcaacctccaggccaacctgtggaaaagttttctgtactcgaacgtggccctgtacctgcccgactcctacaagtacacgccggccaacgtgacgctgcccaccaacaccaacacctacgagtacatgaacggccgcgtggtagccccctcgctggtggacgcctacatcaacatcggcgcccgctggtcgctagaccccatggacaatgtcaaccccttcaaccaccaccgcaatgcgggtctgcgctaccgctccatgcttctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggaaacgacctgcgcgtcgacggcgcctccgtgcgcttcgacagcgtcaacctctatgccacattcttccccatggcgcacaacaccgcctccaccctggaagccatgctgcgcaacgacaccaacgaccagtccttcaacgactacctctcagccgccaacatgctctaccccatcccggccaaggccaccaacgtgcccatctccatcccctcgcgcaactgggccgcctttcgcggctggagtttcacccggctcaagaccaaggaaactccttccctcggctcgggtttcgacccctactttgtctactcgggctccatcccctacctcgacgggaccttctacctcaaccacaccttcaagaaggtctccatcatgttcgactcctcggtcagctggcccggcaacgaccggctgctcacgccgaatgagttcgagatcaagcgcagcgttgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaagccgtcaccctgcccttccagcacaacaactctggcttcaccggctacctcgcacccaccatgcgtcaggggcagccctaccccgccaacttcccctacccgctcatcggctccaccgcagtcccctccgtcacccagaaaaagttcctctgcgacagggtcatgtggcgcatccccttctccagcaacttcatgtccatgggtgccctcaccgacctgggacagaacatgctctatgccaactcggcccacgcgctcgacataacttttgaggtggacccaatggatgagcccaccctcctctatcttctcttcgaagttttcgacgtggtcagagtgcaccagccgcatcgcggcgtcatcgaggccgtctacctgcgcacgcccttctccgccggcaacgccaccacataa

SEQ ID NO: 58 - DNA, 2826 bp, Adenovirus serotype 43 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacgtactttgacatccgcggcgtcctggaccgcggccccagcttcaaaccctactcgggcacggcctacaacagcctggctcccaagggcgcccccaactccagtcagtgggaaacaaaagaaaagcaaaatgggggtagcggagctcagatagaaaaaaatgttacaaaaacatttggagtggctgccatgggaggacttgatataactgatgaaggattgcaaataggagttgaagaaattaataatgtagaagaagaagtttttgctgacaaaatattccagccagagcctcaggtcggagaggaaaactggcaagaaacttttaatttctacggaggcagagctcttaaaaaagacaccaaaatgaagccctgctatggatcatttgcaaggcctacaaatgaaaagggaggacaagctaaacttaaaactggagagaatgtcgacccaacgaaagatttcgacattgatatggctttcttcgatcttaagcaaacagacactggaacaacgcaaaatcagccagatatagttatgtacactgaaaatgtaaaccttgaaactccagacacacatgtggtgtacaaaccaggcaaggaagatgcaagttctgaaattaacttgactcaacagtccatggcaaacagacccaactacattggtttcagagacaactttgtgggtctcatgtattacaacagtactggaaacatgggtgtgctggccggtcaggcttctcagttgaatgctgtggtagacttgcaagacagaaacaccgagctgtcttaccagctattgctagattcccttggagacagaaccagatactttagcatgtggaactctgcggtggacagttatgatccagatgtcaggatcattgagaatcacggtgtggaagatgaacttccaaactattgctttccacttgatggcatgggttcaaatgcggcctaccaaggtgttaagccaaaaactggcaacggatgggatccaaatacagatgttgctgcccagaatcaaatcgccaagggcaacatctatgccatggagatcaacctccaggccaatctgtggaagagttttctgtactcgaacgtggcgctgtacctgcccgactcctacaagtacacaccggccaacgtcactctgcccgccaataccaacacctacgagtacatgaacggccgcgtggtggccccctcgctggtggacgcctacatcaacatcggcgcccgctggtcgctggaccccatggacaacgtcaaccccttcaaccaccaccgcaacgcgggcctgcgctaccgttccatgcttctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggaaacgacctgcgcgtcgacggcgcctccgtccgcttcgacagcgtcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcccatatccatcccctcgcgcaactgggccgccttccgcggctggagtttcacccggctcaagaccaaggaaactccctccctcggttcgggtttcgacccctactttgtctactcgggctccatcccctacctcgacgggaccttctacctcaaccacaccttcaagaaggtctccatcatgttcgactcctcggtcagctggcccggcaacgaccggctgcttacgccgaacgagttcgagatcaagcgcagcgtcgacggggagggctacaacgtggcccaatgcaacatgaccaaggactggttcctcgtccagatgctctctcattacaacatcggttaccagggcttccacgtgcccgagggctacaaggaccgtatgtactccttcttccgaaacttccagcccatgagcaggcaggtggtcgatgagatcaactacaaggactacaaggccgtcaccctgcccttccagcacaacaactcgggcttcaccggctacctcgcacccaccatgcgtcaggggcagccctaccccgccaacttcccctacccgctcatcggccagacagccgtgccctccgtcacccagaaaaagttcctctgcgacagggtcatgtggcgcatccccttctccagcaacttcatgtccatgggtgcccttaccgacctgggtcagaacatgctctacgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacataa

SEQ ID NO: 59 - DNA, 2817 bp, Adenovirus serotype 44 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcttacaacagcctggcccccaagggcgcccccaatccaagtcagtgggaacagaaaaagaatggcggtggcgccgatcaaatggaaacacgcacatatggtgttgccgccatgggaggtattgacattgacaaaaatggacttcaaataggagtcgaacaaacagctgacaatgggcagaaagaaatctatgccgataaattattccagccagagcctcagataggagaggagaattggcatgaaacatttgtttattatggcggcagagctcttaagaaagacaccaagatgaagccttgctatggctcatttgccagacctaccaatgaaaaaggggggcaagctaaaccaaaaactggagaaggagttgacccaaccaaggattttgatatagacctggctttcttcgacattccacaaaatggtgttcaggacaaccatgatccagacatgattatgtatgctgaaaacgtgaatctggaaactccagatactcacatagtttacaagcctggcaaagaggatgagagctctgaagctaacttggtacagcagtctatgcccaacagacccaactacatcggtttcagggacaactttgtagggctcatgtactacaacagcactggcaacatgggtgtgctggctggtcaggcctctcagttgaatgctgtggtcgacttgcaagacagaaacacagagctgtcttaccagcttttgctagattctctgggcgacagaaccagatactttagcatgtggaactctgcggtggacagctatgatcccgatgtcaggatcattgaaaatcacggtgtggaagatgaacttccaaattattgcttcccattggatggggctggaactaatgcggtgtatcaaggtgtaaaagaacaattgaaccaaaatgacaaatgggaagtggataatgcaatatcaaatcagaaccgcatatgcaagggcaatgtctatgccatggagatcaacctccaggccaacctgtggaagagttttctgtactcgaacgtggccctgtacctgcccgactcctacaagtacacgccggccaacgtcacgctgcccaccaacaccaacacctacgagtacatgaacggtcgcgtggtagccccctcgctggtggacgcctacatcaacatcggcgcccgctggtcgctggaccccatggacaacgtcaacccattcaaccaccaccgcaacgcgggcctgcgctaccgttccatgcttctgggcaacggccgctacgtgcccttccacatccaagtgccccaaaagttctttgccatcaagaacctgctcctgctccccggctcctacacctacgagtggaatttccgcaaggacgtcaacatgatcctgcagagttccctcggcaacgacctgcgcgtcgacggcgcctccgtccgcttcgacagcgtcaacctctacgccacctttttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggatggagtttcacccggctcaagaccaaggaaactccctccctcggctcgggtttcgacccctactttgtctactcgggctccatcccatacctcgacgggaccttctacctcaaccacaccttcaagaaggtctccatcatgttcgactcctcggtcagctggccaggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaacaactcgggcttcaccggctaccttgcgcccaccatgcgtcaggggcagccctaccccgccaacttcccctacccgctcatcggccagacagccgtaccctccgtcacccagaaaaagttcctctgcgacagggtcatgtggcgcatccccttctccagcaacttcatgtccatgggcgcccttaccgacttgggtcagaacatgctctatgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacacccttctccgccggcaacgccaccacctaa

SEQ ID NO: 60 - DNA, 2850 bp, Adenovirus serotype 45 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgctcaacactggacgtgggagacaaccgggtgctagacatggccagcacttactttgacatccgcggcgtcctggaccgcggtcccagcttcaaaccctactcgggcacagcttacaacagcctggcccccaagagcgctcccaatcccagccagtgggatgcaaaggaaaaggaaggagttgcccaaacagaaaaaaatgttttaaaaacatttggtgttgccgctacaggtggttttaatattacagatcagggtttgttacttggaactgaggaaacagctgaaaacgttaaaaaggatatctatgcagagaaaactttccagcctgaacctcaagttggtgaagaaaactggcaggaaagtgaagccttttatggaggaagggctattaagaaagacaccaaaatgaagccatgctatggttcatttgccagacccactaatgaaaaaggaggacaggctaaatttaaaacactagatgggcaagttacaaaagatccagatattgactttgcttactttgacgtccctggcggaaaagctccaacaggcagtagtctaccggaagaatacaaagcagatataattttgtacacagaaaatgttaatctggaaacaccagatactcacatagtgtataaacctggcaaagaagatgacaattctgaaattaacttaacacaacagtccatgccaaacagacccaactacattggctttagggacaactttgtaggtctcatgtactacaacagtactggcaacatgggtgtgctggctggtcaggcctctcagttgaatgctgtggtggacttgcaagacagaaacaccgagctgtcttaccagctcttgctagattctctgggtgacagaaccagatactttagcatgtggaactctgcggttgacagttatgatcccgatgtcaggatcattgagaatcacggtgtggaagatgaacttccaaactattgcttcccattgaatggcactggtaccaattccacctatcaaggtgtaaaaattacaggtaataatgatggcgatcttgaaaccgaatgggaaagagatgaagcaatctctagacaaaaccaaatctgcaagggcaacgtctatgccatggagatcaacctccaggccaacctgtggaagagttttctgtactcgaacgtggccctgtacctgcctgactcatacaagtacacgccggccaacgtcacgctgcccgccaacaccaacacctacgagtacatgaacggccgcgtggtagccccctcgctggtggacgcttacatcaacatcggcgcccgctggtcgctggatcccatggacaatgtaaacccattcaaccaccaccgcaacgcgggcctgcgctaccgttccatgttgttgggcaacggtcgctacgtgcccttccacatccaagtgccccaaaagttctttgccatcaagaacctgctcctgctccctggctcctacacctacgagtggaacttccgcaaggacgtcaacatgatcctgcagagttccctcggcaacgacctgcgcgtcgacggcgcctccgtccgcttcgacagcgtcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcacccgtctgaaaaccaaggaaactccctccctcggctcgggtttcgacccatactttgtctactcgggctccatcccctacctcgacgggaccttctacctcaaccacaccttcaagaaggtctccatcatgttcgactcctcggtcagctggcccggcaacgaccggctgctcacaccgaacgagttcgagatcaagcgcagcgtcgacggggagggctacaacgtggcccaatgcaacatgaccaaggactggttcctcgtccagatgctctcccactacaacatcggctaccagggcttccatgtgcccgagggctacaaggaccgcatgtactccttcttccgcaacttccagcccatgagcaggcaggtggtcgatgagatcaactacaaggactacaaggccgtcaccctgcccttccagcacaacaactcgggcttcaccggctacctcgcacccaccatgcgtcaggggcagccctaccccgccaacttcccctacccgctgatcggccagacagccgtgccctccgtcacccagaaaaagttcctctgcgacagggtcatgtggcgcatccccttctccagcaacttcatgtccatgggcgcccttaccgacctgggtcagaacatgctctacgccaactcggctcacgcgctcgatatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacataa

SEQ ID NO: 61 - DNA, 2814 bp, Adenovirus serotype 46 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttaacactggacgtgggcgacaaccgggtgctagacatggccagcacttactttgacattcgcggcgtcctggaccgcggcccaagcttcaaaccctactcgggaacggcttacaacagtctggcccccaagggcgcccccaactccagtcagtgggagcagaaaaaggccaatgggggacctaatgaaatggaaacacacacctttggcgtggctgctatgggaggagaaaacattacaaaagatggtcttcaaataggcaccgaaaccaccgccgagaatcaaaataaagaaatttttgcagacaaaacattccagccagaacctcaagtaggagaggagaactggcaagaaacttttaatttctatggaggcagggctctcaagaaggaaactaaaatgaagccttgctatggatcctttgccagaccaatgaatgaaaaaggaggacaggctaaattcctaacaaaagaaaatggagaattgactgaagatcaggacatagatttgaacttctttgatattaataacccagatacaggaggtgttgcaaatcagcctgacattattatgtatgctgaaaacgtgaatctggaaacaccagatactcacgttgtttacaagtcaggtaaagaggatgacagctctgaagctaacttgctacaacagtccatgccaaacagacccaactacattggcttcagagacaactttgtcggtcttatgtattacaacagtactggcaacatgggtgtgctggctggtcaggcctctcagttgaatgctgtggtcgacttgcaagacagaaacacagagctgtcttaccagctcttgctagattctctgggtgacagaaccagatactttagcatgtggaactctgcggtcgatagctatgatcctgatgtcagaatcattgaaaatcatggtgtggaagatgaacttccaaactattgctttccactgaatggcatgggttcaaatgctgcataccaaggtgttaagccaaaagataacggcggatgggatccaaatactaatgctgcaaggcaaaaccgcattgccatgggaaatgtctatgccatggagatcaacctccaggccaacctgtggaagagttttctgtactcgaacgtggccctgtacctgcccgactcctacaagtacacgccggccaacgtcacgctgcccgccaacaccaacacctacgactacatgaacggccgtgtggtagccccctcgctggtggacgcctacgtcaacatcggcgcccgctggtcgctggaccccatggacaatgtcaaccccttcaaccaccaccgcaacgcgggcctgcgctaccgttccatgctgttgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggaaacgatctgcgcgtcgacggcgcctccgtccgcttcgacagcgtcaacctctacgccaccttcttccccatggcgcacaacaccgcctccaccctggaagccatgctgcgcaacgacaccaacgaccagtccttcaacgactacctctcgggcgccaacatgctctaccccatcccggccaaggccaccaacgtgcccatctccatcccctcgcgcaactgggccgccttccgcggctggagtttcacccggctcaagaccaaggaaactccctccctcggctcgggtttcgacccctactttgtctactcgggctccatcccctacctcgacgggaccttctacctcaaccacaccttcaagaaggtctccatcatgttcgactcctcggtcagctggcccggcaacgaccggctgcttacgccgaacgagttcgagattaagcgcagtgtcgacggggagggctacaacgtggctcaatgcaacatgaccaaggactggttcctcgtccagatgctctcccactacaacatcggctaccagggcttccatgtgcccgagggctacaaggaccgcatgtactccttcttccgaaacttccagcccatgagcaggcaggtggtcgatgagatcaactacaaggactacaaggccgttaccctgcccttccagcacaacaactcgggcttcaccggctacctcgcacccaccatgcgccaggggcagccctaccccgccaacttcccctacccgctcatcggctccaccgcagtgccctccgtcacccagaaaaagttcctctgcgacagggtcatgtggcgcatccccttctccagcaacttcatgtccatgggcgccctcaccgacctgggtcagaacatgctctacgccaactcggcccatgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggaaacgccaccacataa

SEQ ID NO: 62 - DNA, 2841 bp, Adenovirus serotype 47 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggccccagcttcaaaccctactcgggcacggcttacaacagcctggcccccaagggcgcccccaatcccagtcaatgggatgctcaagaaaataatgctggagatgctaagactcatacatatggtgtagcttccatgcctggaattgatattacagacaaaggtcttcaaattggaattgacgccaacaaagatgaagatgaaggcaatgaaatttttgctgacaaaacattccagccagagcctcaagtgggggaggaaaattggcaagaaagcgaaaacttttatggaggcagggcgcttaaaaaagaaaccaaaatgaagccttgctatggctcatttgccagacctaccaatgaaaaaggcggacaggctaaattcaaaacgcctgataaagaaggagagcaaccaaaagaatatgacatagatatgaatttctttgatattccaaacaccggaacaggcggcaatggtacaaatgtcaataacaaaccagacattgtactttatgctgagaatatgaatttggaaactccagacacacatgtggtgtacaaaccaggaaaagaggatgacagttcagaagttaacctcacacagcagtccatgcccaacagacccaactacattggcttcagggacaactttgtaggtctcatgtattacaacagcactggcaacatgggtgtgctggctggtcaggcctctcagttgaatgctgtggttgacttgcaagacagaaacaccgagctgtcttaccagctcttgctagattctctgggtgacagaaccagatactttagcatgtggaactctgcggtggacagctatgatcccgatgtcaggatcattgaaaatcacggtgtggaagatgaacttccaaactattgcttcccattggatggcactggaactgcctctgcataccagggtgttaaagtaaaaaatgggactgttgctggcaaagtggaatgggatccagacacaaaagttgcttcaaagaacagaatatgcaagggcagcatctttgccatggagatcaacctccaggccaacctgtggaagagttttctgtactcgaacgtggcactgtacctgcccgactcctacaagtacacgccagccaacgtcacgctgcccgccaacaccaacacctacgagtacatgaacggccgcgtggtggccccctcgctggtggacgcctacgtcaacatcggcgcccgctggtcgctggaccccatggacaacgtcaaccccttcaaccaccaccgcaacgcgggcctgcgctaccgttccatgctcctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggaaacgacctgcgcgtcgacggcgcctccgtccgcttcgacagcgtcaacctctacgccaccttcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcacccgtctcaagaccaaggaaactccctccctcggctcgggtttcgacccctactttgtctactctggctccatcccatacctcgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcacactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaacaactcgggcttcaccggctacctagcacccaccatgcgtcaggggcagccctaccccgccaacttcccctacccgctcatcggctccgccgcagtaccctccgtcacccagaaaaagttcctctgcgacagggttatgtggcgcatccccttctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacataa

SEQ ID NO: 63 - DNA, 2832 bp, Adenovirus serotype 49 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggctcccacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcttataacagcctggcccccaagggcgcccccaactccagtcagtgggacgctaaagaaaataatggtcagggagaagctaagactcatacatatggtgtggctgccatgggcggttataatatcacaaaggatggactgcaaataggaatagatgaaaacaaggaagaagatgaagaaggaagagaaattttcgcagttaaatcataccagccagaacctcaagtgggagaggaaaactggcaaaacacagaaaacttctacggaggcagagctcttaaaaaagaaaccaaaatgaaaccatgctatggttcttttgccagacccaccaatgataagggaggtcaggctgtttttaaaactggtgaaaatggaaagcctaccgaagagctagatatcgatcttgctttctttgatctcagacaaaatgacactggtggaaataacaatcaaccagatatgatcatgtatgctgaaaatgtaaacctggaaacaccagacactcatgtggtgtacaaacctggaacttcagatgacagttccgaaattaacttatgtcagcaatccatgcccaacagacctaactacattggatttagagataactttgttgggctgatgtactacaacagcactggcaacatgggtgtgctagcgggtcaggcctctcagttgaatgctgtggtcgacttgcaagacagaaacaccgagctgtcttaccagctcttgctagattctctgggtgacagaacaaggtactttagcatgtggaactctgcggtggacagctatgatcccgatgtcaggatcattgagaatcacggtgtggaagatgaacttccaaactattgctttccattggatggctctggcagcagtactgcttaccaaggtgttgaacctgatacaactgttgctggtacaaacgacaaatggaaagtaaatgctaaagtcgctcagcataatcagatcgccaagggcaacctgttcgccatggagatcaacctccaggccaacctgtggaagagttttctgtactcgaacgtggccctgtacctgcccgactcatacaagtacacgccggccaacgtcaagctgcccaccaacaccaacacctacgactacatgaacggccgcgtggtagccccctcactggtggacgcctacatcaacatcggcgcccgctggtcgctggatcccatggacaatgtcaaccccttcaaccaccaccgcaacgcgggcctgcgctaccgctccatgcttctgggcaacggccgctacgtgcccttccacatccaagtgccccaaaagttctttgccatcaagaacctgctcctgctcccgggctcctacacctacgagtggaacttccgcaaggatgtcaacatgatcctgcagagttccctcggcaacgacctgcgcgtcgacggcgcctccgtccgcttcgacagcgtcaacctctacgccaccttcttccccatggcgcacaacaccgcctccaccttggaagccatgctgcgcaacgacaccaacgaccagtccttcaacgactacctctcggccgccaacatgctctaccccatcccggccaaggccaccaacgtgcccatctccatcccctcgcgcaactgggccgccttccgcggctggagtttcacccggctcaagaccaaggaaactccttccctcggctcgggtttcgacccctactttgtctactcgggctccatcccctacctcgacgggaccttctacctcaaccacaccttcaagaaggtttccatcatgttcgactcctcggtcagctggcccggcaacgaccggctgctcacgccgaacgagtttgagatcaagcgcagcgtcgacggggagggctacaacgtggcccaatgcaacatgaccaaagactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggcggtcaccctgccattccagcacaacaactcgggcttcaccggctacctcgcccccaccatgcgccaggggcagccctaccccgccaacttcccctacccgctcatcggctccaccgcagtcccctccgtcacccagaaaaagttcctctgcgacagggtcatgtggcgcatccccttctccagcaacttcatgtccatgggcgccctcaccgacctgggtcagaacatgctatacgccaactcggcccacgcgcttgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggcaacgccaccacataa

SEQ ID NO: 64 - DNA, 2841 bp, Adenovirus serotype 50 Hexon gene
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcatacaaggcgcgattcactctggccgtgggcgacaaccgggtgctagacatggccagcacttactttgacatccgcggcgtcttggaccgcggccccagcttcaaaccctactcgggcacagcttacaacagtctggctcccaagggcgcccccaactccagtcagtgggagcagaaaaaaactactggtggaggcaatgacatggaaacacacacctttggcgtggctgctatgggaggagaaaacattacaaaagatggtcttcaaattggaactgacacaacagcagatgctgataaaccaatatatgcagacaaaacattccagccagaacctcaagtaggagaggagaactggcaagaaacttttaatttctatggaggaagggctcttaagaaggacaccaagatgaaaccatgctatggttcatttgccagacctactaatgaaaaaggaggacaggccaaacttaaaaatggaccagatggcaaacccacaaaagagtttgacatagatttggctttcttcgacactcctggcggcaaacttccaggcaatgatcaaaaggaagaatacaaagcagatattgtcatgtacactgagaatgcttatctggaaacgccagacactcatgtagtatacaaaccaggcaaggatgatgcgagttcagaggccaacttggttcagcaatccatgccaaaccggcccaactacattggcttcagggacaactttgtaggtttgatgtattacaacagcactggcaacatgggtgtgctggctggtcaggcctctcagttgaatgctgtggtcgacttgcaagacagaaacaccgagctgtcttaccagctcttgctagattctctgggtgacagaaccagatactttagcatgtggaactctgcggtggacagctatgatcccgatgtcaggatcattgagaatcacggtgtggaagatgaacttcccaactattgcttcccattggatggctctggcaccaatgccgcctatcaaggtgtaaaggtgacagaaggtgaggatggagctgaggaaagcgaatgggaacttgaaaacaaactcgctgcccgtaaccaaatatgcaagggcaacatctttgccatggaaatcaacctccaggccaacctgtggagaagttttctgtactcgaacgtggccctgtacctgcccgactcctacaagtacactccggccaacgtcacgctgcccgccaacaccaacacctacgagtacatgaacggccgcgtggtagccccctcgctggtggacgcctacatcaacataggcgcgcgctggtcgctggaccccatggacaacgtcaatcccttcaaccaccaccgcaatgcgggtctgcgctaccgctccatgcttctgggcaacggccgctacgtgcccttccacatccaagtgccccaaaagttctttgccatcaagaacctgctcctgctccccggctcctacacctacgagtggaacttccgcaaggacgttaacatgatcctgcagagttccctcggcaacgacctgcgcgtcgacggcgcctccgtccgcttcgacagcgtcaacctctatgccaccttcttccccatggcgcacaacaccgcctccaccttggaagccatgctgcgcaacgacaccaacgaccagtccttcaacgactacctctcggccgccaacatgctctaccccatcccggccaaggccaccaacgtgccaatctccatcccctcgcgcaactgggccgccttccgcggctggagtttcacccggctcaagaccaaggaaactccctccctcggctcgggtttcgacccctactttgtctactcgggatccatcccctacctcgacgggaccttctacctcaaccacaccttcaagaaggcctccataatgttcgactcctcggtcagctggcccggcaacgaccggctgcttacgccgaacgagttcgagatcaagcgcagcgtcgacggggagggcttcaacgtggcccaatgcaacatgaccaaggactggttcctcgtccagatgctctcccactacaacatcggctaccagggcttccatgtgcccgagggctacaaggaccgcatgtactccttcttccgcaacttccagcccatgagcaggcaggtggtcgatgagatcaactacaaggactacaaagccgtcactctgcccttccagcacaacaactcgggcttcaccggctacctggctcccaccatgcgtcaggggcaaccctaccccgccaacttcccctatccgctcatcggctccaccgcagtcccctccgtcacccagaaaaagttcctctgcgacagggtcatgtggcgcatccccttctccagcaacttcatgtccatgggcgccctcaccgacctgggtcagaacatgctctacgccaactcggcccatgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggaaacgccaccacataa

SEQ ID NO: 65 - DNA, 2823 bp, Adenovirus serotype 51 Hexon gene
atggccaccccatcgatgctgccccagtgggcatacatgcacatcgccggacaggatgcttcggagtacctgagtccgggtctggtgcagttcgcccgcgccacagacacctacttcaatctggggaacaagtttaggaaccccaccgtggcgcccacccatgatgtgaccaccgaccgcagtcagcggctgatgctgcgctttgtgcccgttgaccgggaagacaatacctacgcatacaaagttcgatacaccttggctgtgggcgacaacagagtgctggatatggccagcactttctttgacattcggggtgtgttggatagaggtcctagcttcaagccatattctggcactgcttacaactcattggcccctaagggcgctcccaatacatctcagtggcttaataagggagatgaagaggatggggaagacgaccaacaagctacatacacttttggcaatgcgccagtaaaagccgaagctgaaattacaaaagaaggactgccaataggtttggaagttccatctgaaggtggccctaaacccatttatgctgataaactgtatcagccagaacctcaggtgggagaggaatcttggactgatacggatggcacagatgaaaaatatggaggcagagcacttaaacctgaaactaaaatgaaaccctgctacgggtctttcgctaaacctactaatgttaaaggcgggcaggcaaaagtgaagaaagaagaagaaggcaaggttgaatatgacattgacatgaactttttcgacctaagatcacaaatgactggcctcaagcctaaaattgtaatgtatgcagaaaatgtggatctagaaactcctgacactcatgtggtgtacaaacctggagcttcagatgctagctctcatgcaaaccttggtcaacagtccatgcccaatagacctaactatattggcttcagggacaacttcatcggactcatgtactacaacagtactggcaacatgggagtgctggctggacaagcgtctcagctaaatgcagtggttgacttgcaagacagaaacacagaattgtcatatcaactcttgcttgattctctgggggacagaaccagatatttcagtatgtggaatcaagcagtggatagctatgacccagatgtgcgtgttattgagaaccatggtgtggaagatgaacttcctaactattgttttccattggatggtgtaggtccgcgaatagacagttacaagggaattgagacaaatggtgatgaaaccactacttggaaagatttagagccaaagggcataagtgaaattgctaagggaaatccgtttgccatggaaattaacctccaagctaatctctggagaagttttctttattccaatgtggctctgtatctcccagactcctacaaatacaccccagccaatgtcactcttccaactaacaccaacacttatgactacatgaatgggcgggtggttcccccatccctggtggatacctacgtaaacattggcgccagatggtctttggatgccatggacaatgtcaacccctttaaccatcaccgcaacgctggcctgcgataccggtccatgcttttgggcaatggtcgttacgtgcctttccacattcaagtgcctcagaaattctttgctgtgaagaacctgctgcttctacccggttcttacacctacgagtggaacttcagaaaggatgtgaacatggtcctgcagagttcccttggtaatgatctccgggtcgatggtgccagcataagttttaccagcatcaatctctatgccaccttcttccccatggcccacaacactgcctccacccttgaagccatgctgcgcaatgacaccaatgatcaatcattcaatgactacctttctgctgccaacatgctctaccccatcccggccaacgctaccaacgttcccatctccattccctctcgcaactgggccgccttcagaggctggtccttcaccagactcaaaaccaaggagactccctctttgggatcagggttcgatccctactttgtttactctggttctataccctacctggatggtaccttctaccttaaccacactttcaagaaagtctccatcatgtttgactcttcagtgagctggcctggtaatgacagattgctaagtccaaatgagttcgaaatcaagcgcacagttgatggggaaggctacaatgtggcccaatgtaacatgaccaaagactggttcctggtccagatgcttgccaactacaacattggataccagggcttctacgttcctgagggttacaaggatcgcatgtactccttcttcagaaacttccagcccatgagtagacaggtggttgatgagattaactacaaagactataaagctgtcgccgtaccctaccagcataataactctggctttgtgggttacatggctcctaccatgcgtcagggtcaagcgtaccctgctaactacccataccccctaattggaaccactgcagtaaccagtgtcacccagaaaaaattcctgtgcgacaggaccatgtggcgcatcccattctctagcaacttcatgtccatgggtgcccttacagacctgggacagaacttgctgtatgccaactcggcccatgcgctggacatgacttttgaggtggatcccatggatgagcccaccctgctttatcttcttttcgaagtcttcgacgtggtcagagtgcaccagccacaccgcggcgtcatcgaggccgtctacctgcgcacaccgttctcggccggcaacgccaccacataa

SEQ ID NO: 66 - protein, 963 aa, Adenovirus serotype 6 Hexon
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNSCEWEQNETAQVDAQELDEEENEANEAQAREQEQAKKTHVYAQAPLSGIKITKEGLQIGTADATVAGAGKEIFADKTFQPEPQVGESQWNEADATAAGGRVLKKTTPMKPCYGSYARPTNSNGGQGVMVEQNGKLESQVEMQFFSTSTNATNEVNNIQPTVVLYSEDVNMETPDTHLSYKPKMGDKNAKVMLGQQAMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGIGITDTFQAVKTTAANGDQGNTTWQKDSTFAERNEIGVGNNFAMEINLNANLWRNFLYSNIALYLPDKLKYNPTNVEISDNPNTYDYMNKRVVAPGLVDCYINLGARWSLDYMDNVNPFNHPRHAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGIIHQHNNSGFVGYLAPTMREGQAYPANVPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 67 - protein, 943 aa, Adenovirus serotype 8 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNPSQWEQAKTGAGVDQNQKETRTYGVAATGGYNITKEGLQIGIDETKEDPNNKIYADKTFQPEPQIGENNWQDTNVFYGGRALKKETKMKPCYGSFARPTNKKGGQAKVLTTEDGQPTENFDIDLAFFDIPQAGGNDNLDPDMILYAENVNLETPDTHVVYKPGKDDASSAANLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGTGTNATYQGVEPDNAQGQNDKWKKDEKVAAQNQICKGNIYAMEINLQANLWKSFLYSNVALYLPDSFKYTPANVTLPTNTNTYEYMNGRVAAPSLVDAYVNIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGTIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVALPFQHNNSGFTGYLAPTLRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 68 - protein, 945 aa, Adenovirus serotype 9 Hexon
MATPSMMPQWAYMHIAGQDTSEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWLAKDTNAGDQALKTHTHGVAAMGGTDITAKGLQIGVDTTENKNEPIYANEIYQPEPQVGEENLQDVENFYGGRALKKETKMKPCYGSFARPTNEKGGQAKIFNLTAIGQPTKNHDITMNFFDTPGGTVGQDTELEADIVMYAENVHLETPDTHVVYKPGTSDESSEANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGAGTNATYQGVKVKNGQDGDVNADWEKDPNLASRNQICKGNIFAMEINLQANLWKSFLYSNVALYLPDSYKYTPANVTLPANTNTYEYMNGRVVAPSLVDAYINIGARWSLDPMDNVNPFNHHRNAGLRYRSMLMGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 69 - protein, 950 aa, Adenovirus serotype 10 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKSAPNPSQWTANEKQTGGQPKSVTQTFGSAPMGGSNITIEGLVIGTKEEEGNATEEIFADKTFQPEPQVGEENWQETEAFYGGRALKKDTKMKPCYGSFARPTNEKGGQAKLKLNAQGQPTKDYDIDLAFFDTPGGTPPTGSGQQEEYKADIIMYTENVNLETPDTHVVYKPGKEDESSETNLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLNGTGTNSTYQGVKVKTGQDGAEETEWDKDETVARQNQIAKGNVYAMEINLQANLWKSFLYSNVALYLPDSYKYTPANVTLPANTNTYEYMNGRVVAPWLVDAYINMGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALEMTFEVDPMDEPTLLYLLFEGFDVVRVHQPPSGVIEAVYLRTPFSAGNATTZ

SEQ ID NO: 70 - protein, 939 aa, Adenovirus serotype 13 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNPSQWTTKEKQTGVNAGDKEVTKTFGIAAMGGSNISENGLQIGTDTTADGTKPIYADKTFQPEPQVGEENWQDNDEYYGGRALKKDTKMKPCYGSFAKPTNKEGGQAKLKETPNGADPQYDVDMAFFDSTTINIPDVVLYTENVDLETPDTHVVYKPGKEDDSSEVNLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGIQTNSAYQGVKLKDNPTGGGANGDWVKDDTISAHNQIGKGNIFAMEINLQANLWKSFLYSNVALYLPDSYKYTPANVTLPANTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAGNMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATT

SEQ ID NO: 71 - protein, 945 aa, Adenovirus serotype 14 Hexon
MATPSMLPQWAYMHIAGQDASEYLSPGLVQFARATDTYFNLGNKFRNPTVAPTHDVTTDRSQRLMLRFVPVDREDNTYSYKVRYTLAVGDNRVLDMASTFFDIRGVLDRGPSFKPYSGTAYNSLAPKGAPNASQWLDKGVETTEERQNEDGENDEKATYTFGNAPVKADADITKDGLPIGLEVPAEGDPKPIYANKLYQPEPQVGQESWTDTDGTEEKYGGRVLKPDTKMKPCYGSFAKPTNVKGGQAKVKTEEAGNIEYDIDMNFFDLRSQKQGLKPKIVMYAENVDLESPDTHVVYKPEVSDASSNANLGQQSMPNRPNYIGFRDNFIGLMYYNSTGNMGVLAGQASQLNAVVDLQDRNTELSYQLLLDSLGDRTRYFSMWNQAVDSYDPDVRVIENHGVEDELPNYCFPLDGIGPRTDSYKEIQLNGDQAWKDVNPNGISELVKGNPFAMEINLQANLWRSFLYSNVALYLPDSYKYTPSNVTLPENKNTYDYMNGRVVPPSLVDTYVNIGARWSLDAMDNVNPFNHHRNAGLRYRSMLLGNGRYVPFHIQVPQKFFAVKNLLLLPGSYTYEWNFRKDVNMVLQSSLGNDLRVDGASISFTSINLYATFFPMAHNTASTLEAMLRNDTNDQSFNDYLSAANMLYPIPANATNIPISIPSRNWAAFRGWSFTRLKTKETPSLGSGFDPYFVYSGSIPYLDGTFYLNHTFKKVSIMFDSSVSWPGNDRLLSPNEFEIKRTVDGEGYNVAQCNMTKDWFLVQMLANYNIGYQGFYIPEGYKDRMYSFFRNFQPMSRQVVDEVNYKDFKAVAIPYQHNNSGFVGYMAPTMRQGQPYPANYPYPLIGTTAVNSVTQKKFLCDRTMWRIPFSSNFMSMGALTDLGQNMLYANSAHALDMTFEVDPMDEPTLLYLLFEVFDVVRVHQPHRGIIETVYLRTPFSAGNATT

SEQ ID NO: 72 - protein, 941 aa, Adenovirus serotype 15 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWEQKKANAGDQKETHTYGVAPMGGENITISGLQIGTDTTNGKQDPIYANKLYQPEPQVGEENWQETEAFYGGRALKKETKMKPCYGSFARPTNEKGGQAKLRDPEKSQEDFDIVLAFFDTPGGTLTGGGTEYKADIVMCTENVNLETRDTHVVYKPGKDDDSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNAAYEGVKVKNGEDGDQESEWEKDTNVADRNQICKGNIYAMEINLQANLWKSFLYSNVALYLPDSYKYTPANVTLPANTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATT

SEQ ID NO: 73 - protein, 920 aa, Adenovirus serotype 18 Hexon
MATPSMMPQWSYMHIAGQDASEYLSPGLVQFARATDTYFTLGNKFRNPTVAPTHDVTTDRSQRLTLRFVPVDREDTAYSYKARFTLAVGDNRVLDMASSYFDIRGVLDRGPSFKPYSGTAYNSLAPKGAPNASQWLTTNDNKSHTFAQAPYIGSSITKDGIQVGTNTANPPQPVYADKTYQPEPQVGESQWNAPLPNNAKAAGRVLKNTTPMYPCYGSYARATNENGGQSKNNEGVEMQFFASAADNQNDPSVVLYSEDVNLEAPDTHIVFKPAVAADTVSSELLLGQQAAPNRPNYIGFRDNFIGLMYYNSTGNMGVLAGQASQLNAVVDLQDRNTELSYQLMLDALGDRSRYFSMWNSAVDSYDPDVRIIENHGVEDELPNYCFPLGAVGIVNSYKGVKYDANAWAKDNNKADNNNLATGNIFAMEINLQANLWRSFLYSNVGLYLPDSLKYTPGNIKLPDNKNTYEYMNGRVTAPGLVDTYVNIGARWSPDVMDNINPFNHHRNAGLRYRSMLLGNGRFVPFHIQVPQKFFAIRNLLLLPGSYTYEWNFRKDVNMILQSTLGNDLRVDGASIRFDNIALYANFFPMAHNTASTLEAMLRNDTNDQSFNDYLCAANMLYPIPANATSVPISIPSRNWAAFRGWSFTRLKTKETPSLGSGFDPYFVYSGTIPYLDGTFYLNHTFKKVSIMFDSSVSWPGNDRLLTPNEFEIKRSVDGEGYNVAQCNMTKDWFLIQMLSHYNIGYQGFYVPEGYKDRMYSFFRNFQPMSRQVVDTTEYKDYKQVTVKFQHNNSGFVGYLGPTMREGQAYPANYPYPLIGKTAVESITQKKFLCDRVMWRIPFSSNFMSMGALTDLGQNMLYANSAHALDMTFEVDPMDEPTLLYVLFEVFDVVRIHQPHRGVIEAVYLRTPFSAGNATTZ

SEQ ID NO: 74 - protein, 952 aa, Adenovirus serotype 19 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWDAQEKNGQGGNDMVTKTHTFGVAAMGGTNITNQGLLIGTEETAHNPPKEIFADKLFQPEPQVGEENWQDTNAFYGGRALKKETKMKPCYGSYARPTNTSGGQAKLKTGDNIDPTKDFDIDLAFFDTPGGNPPAGGSGTEEYKADIVMYTENVNLETPDTHVVYKPGKEDESSEANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNAAYQGVKVQDGEDGDKETEWEKDTKVADRNQLCKGNIFAMEINLQANLWKSFLYSNVALYLPDSYKYTPANVTLPANTNTYEYMNGRVVAPSLVDAYVNIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 75 - protein, 944 aa, Adenovirus serotype 20 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWLAKDTNAAGQPDKTHTYGVAAMGGEDITEKGLQIGIDETKEENNKIFANEIYQPEPHVGEENWQETFVFYGGRALKKDTKMKPCFGSFARPYYEKGGQAKFVLDQEGKPTKNHDITMAFFDTPGGQLNGKDELKADIVMYTENVNLETPDTHVVYKPGKEDDSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGAGTNAVYQGVKITDGNDGDVNDDWEKDTAVSERNQICKGNIYAMEINLQANLWKSFLYSNVGLYLPDSYKYTPANVTLPANTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 76 - protein, 936 aa, Adenovirus serotype 22 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWAQKKTGEDNQTETRTFGVAAMGGILIDKNGLQIGTDETKPNNKEVYADKTFQPEPQIGEENWQDGDVFYGGRTIKKETKMKPCYGSFARPTNEKGGQAKFKTNAEGQPTEELDIDLNFFDINGGAGDNEFNPDMVMYAENMNLETPDTHVVYKPGTSDDSSEANLAQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGTGTNSTYQGVKETAAQNGWEKDPNVAAQNQICKGNIYAMEINLQANLWKSFLYSNVALYLPDSFKYTPANVTLPTNTNTYEYMNGRVVAPSLVDAYVNIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 77 - protein, 932 aa, Adenovirus serotype 23 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNPSQWEQKKTGVGPEAMEKHTFGMAAMAGEAITNKGLQIGVDTTDGKQDPIYANQLYQPEPQVGEDSWNDDVAPSYGGRALKKETKMKPCYGSFARPTNEKGGQAKLRDPEKSQEDFDIDMAFFDSNTINTPDVVLYTENVNLETPDSHVVYKAGTSDESSEINLGQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGVATNTVYQGVKLQTGQTDKWQKDDDVSTQNRIGKGSVFAMEINLQANLWKSFLYSNVALYLPDSFKYTPANVTLPTNTNTYEYMNGRVVAPSLVDAYVNIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSLMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 78 - protein, 937 aa, Adenovirus serotype 24 Hexon
MATPSMMPQWAYMHIAGQDASEYLSPGLVQFARATDTYFSLGNKFRNPTVPPTHDVTTDRSQRLTLRFVPVDREDTRYLYKARFTLAVGDNRVLDMASTYFDIRGVLDRGPSFKPYSGTAYNSLAPKGAPNSSQWEQAKATNAGQKETHTFGVAAMGGEDITVKGLQIGTDETKEDGEDEIFADQTFQPEPQVGEQNWQETFVFYGGRALKKETKMKPCYGSYARPTNEKGGQAKFTLDEKGQPTKIPDITMDFFDSPQDDTSGVTNKPDIVMYAENVNLEAPDTHVVYKPGKDDSSSSANLTQQAMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLNGSGSNSTYKGVKAGTGNNWDDDENVARQNQIGTGNLFAMEINLQANLWKSFLYSNVALYLPDSYKYTPANVTLPTNTNTYDYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 79 - protein, 946 aa, Adenovirus serotype 25 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWEEKKSGARNQTETHTFGVAPMGGTNITISGLQIGTEEEDGNPTKEIFADKTFQPEPQIGEENWQDTENFYGGRALKKDTKMKPCYGSFARPTNEKGGQAKLKLDAQGQSTKDYDIDLAFFDSPGGNTTAGGQEELKADIVMYTENAYLETPDTHVVYKPGTSDDSSAANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNSAYQGVKVKNGEDGDIESEWEKDTNVAARNQLCKGNIFAMEINLQANLWKSFLYSNVALYLPDSYKYTPANVKLPTNTNTYEYMNGRVVAPSLVDAYVNIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSINLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 80 - protein, 946 aa, Adenovirus serotype 26 Hexon; identical to SEQ ID NO: 79

SEQ ID NO: 81 - protein, 937 aa, Adenovirus serotype 27 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWTAKENQKDVTKTFGVAPMGGINISKDGLQIGVEETVDKQEKEIYADKSFQPEPPVGKENWQESEAFYGGRALKKDTKMKPCYGSFARPTNEKGGQAKFKAPAVEGEQPKELDIDFAFFDTDSGDTEYKADIVMYAENVNLETPDTHVVYKPGKEDDSSEANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNSTYQGVKITDGNGALQNGWAKDDAISRQNQICKGNIYAMEINLQANLWKSFLYSNVALYLPDSFKYTPANVTLPTNTNTYEYMNGRVVAPSLVDAYINIGARWALDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 82 - protein, 944 aa, Adenovirus serotype 28 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWDAQEKSGQGSDMVTKTHTFGVAAMGGENITKNGLQIGTEITADNQKKEIFANKTYQPEPQVGEENWQENEVFYGGRALKKETKMKPCYGSFARPTNENGGQAKFKTPAEGQEPKELDIDLAFFDTDGGTADTEYKADIVMYAENVNLETPDTHVVYKPGKEDDSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGLGTNATYQGVKVSTGDGATQSGWAKDDTMARQNQICRGNIYAMEINLQANLWKSFLYSNVALYLPDSYKYTPANVTLPANTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 83 - protein, 942 aa, Adenovirus serotype 29 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWEQKKANAGDQKETHTYGVAPMGGENITISGLQIGTDTTNGKQDPIYANKLYQPEPQVGEENWQETEAFYGGRALKKETKMKPCYGSFARPTNEKGGQAKLRDPEKSQEDFDIALAFFDTPGGTLTGGGTEYKADIVMCTENVNLETPDTHVVYKPGKDDDSSEINLGQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNAAYEGVKVKNGQDGDQESEWEKDTNVADRNQICKGNIYAMEINLQANLWKSFLYSNVALYLPDSYKYTPANVTLPTNTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFLPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFYVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 84 - protein, 942 aa, Adenovirus serotype 30 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWEQKKANAGDQKETHTYGVAPMGGENITISGLQIGTDTTNGKQDPIYANKLYQPEPQVGEENWQETEAFYGGRALKKETKMKPCYGSFARPTNEKGGQAKLRDPEKSQEDFDIDLAFFDTPGGTLTGGGTEYKADIVMCTENVNLETPDTHVVYKPGKDDDSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNAAYEGVKVKTGQDGDQETEWEKDTNVADRNQICKGNIYAMEINLQANLWKSFLYSNVALYLPDSYKYTPANVTLPTNTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFVGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 85 - protein, 923 aa, Adenovirus serotype 31 Hexon
MATPSMMPQWSYMHIAGQDASEYLSPGLVQFARATDTYFTLGNKFRNPTVAPTHDVTTDRSQRLTLRFVPVDREDTAYSYKARFTLAVGDNRVLDMASSYFDIRGVLDRGPSFKPYSGTAYNSLAPKGAPNASQWLTTNNGNKTHTFAQAPYIGDSISKDGIQVGTNTANPQQAVYADKTYQPEPQVGESQWNASVSNNAKAAGRVLKSTTPMYPCYGSYARATNEKGGQSKNNDGVEMRFFASSANDNQNDPTVVMYSEDVNLEAPDTHIVYKPAVTADTISSELLLGQQAAPNRPNYIAFRDNFIGLMYYNSTGNMGVLAGQASQLNAVVDLQDRNTELSYQLMLDALGDRSRYFSMWNSAVDSYDPDVRIIENHGVEDELPNYCFPLGAVGVVNSYKGVKQNDGNGWVKDDSVADTNDIAKGNIFAMEINLQANLWRSFLYSNVGLYLPDDLKYTPGNIKLPENKNTYEYMNGRVTVPGLVDTYVNIGARWSPDVMDNINPFNHHRNAGLRYRSMLLGNGRFVPFHIQVPQKFFAIKNLLLLPGSYTYEWNFRKDVNMILQSTLGNDLRVDGASIRFDNIALYANFFPMAHNTASTLEAMLRNDTNDQSFNDYLCAANMLYPIPANATSVPISIPSRNWAAFRGWSFTRLKTKETPSLGSGFDPYFVYSGTIPYLDGTFYLNHTFKKVSIMFDSSVSWPGNDRLLTPNEFEIKRSVDGEGYNVAQCNMTKDWFLVQMLSHYNIGYQGFYVPESYKDRMYSFFRNFQPMSRQVVDTTEYKEYKKVTVEFQHNNSGFVGYLGPTMREGQAYPANYPYPLIGKTAVQSVTQKKFLCDRVMWRIPFSSNFMSMGAVTDLGQNMLYANSAHALDMTFEVDPMEEPTLLYVLFEVFDVVRIHQPHRGVIEAVYLRTPFSAGNATTZ

SEQ ID NO: 86 - protein, 946 aa, Adenovirus serotype 32 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWAAKDANTADQTVKTHTHGVAAMGGTDITAKGLQIGVDTTENNAGPIYANEIYQPEPQIGEENLQDVENYYGGRALKKETKMKPCYGSFARPTNEKGGQAKFITGEDGQPTKNHDITMNFFDTPGGTIGQADELEADIVMYAENVHLETPDTHVVYKPGTSDESSEANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGAGTNATYQGVKVKDGEDGNENADWVKDPNLASRNQICKGNIFAMEINLQANLWKSFLYSNVALYLPDSYKYTPANITLPTNTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTKAZ

SEQ ID NO: 87 - protein, 944 aa, Adenovirus serotype 33 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWEQKKATGGADAKETHTFGVAAMGGLNITDKGLQIGIDEDNVDGDDEIYADKTFQPEPQVGEENWKESFNFYGGRAIKKDTKMKPCYGSFARPTNVKGGQAKLKTGEDIDPNKDFDIDMAFFDLKQADTGGNNNQADVVMYTENIHLETPDTHVVYKPGKEDASSEINLTQQSMANRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGSSTAFQGVKVKTPAGTGQNTVWEVNNDAATHNQIARGNLYAMEINLQANLWKSFLYSNVALYLPDSYKYTPANVTLPTNTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 88 - protein, 945 aa, Adenovirus serotype 36 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWTDKERQNGGQPPTTKDVTKTFGVAARGGLHITDKGLQIGEDENNEDGEEEIYADKTFQPEPQVGEENWQDTDVFYGGRALKKETKMKPCYGSFARPTNEKGGQAKFLNGENGQPSKDQDITLAFFDLKQNDTGTTQNQPDVVMYTENVYLETPDTHVVYKPGKEDTSSAANLTQQSMPNRPNYIGFRDNFVGLMYYNSTGNMGVLAGQASQLNAVVDLQDRNTELSYQLLLDSLGDRTRYFSMWNSTVDSYDPDVRIIENHGVEDELPNYCFPLDGSGSNTAYQGVKYENGAGNGSWKVDGEVASQNQIAKGNLYAMEINLQANLWKSFLYSNVALYLPDSYKYTPANITLPTNTNTYEYMNGRVVAPSLVDAYVNIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATLFPMARNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFYVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 89 - protein, 940 aa, Adenovirus serotype 37 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNPSQWTTKEKQNGGTGAEKDVTKTFGLAAMGGSNISKDGLQIGTDKTANAEKPIYADKTFQPEPQVGEENWQDNDEYYGGRALKKDTKMKPCYGSFAKPTNKEGGQAKLKETPNGTDPQYDVDMAFFDSSTINIPDVVLYTENVDLETPDTHVVYKPGKEDDSSEANLTQQSMPNRPNYIGFRDNFVGLLYYNSTGNMGVLAGQASQLNAVVDLQDRNTELSYQLLLDSLGDRTRYFSMWNSAVDSYDPDVRIIENHGVEDELPNYCFPLDGVQTNSAYQGVKLKPDQTGGGVNGDWVKDDDISAHNQIGKGNIFAMEINLQANLWKSFLYSNVALYLPDSYKYTPANVTLPANTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 90 - protein, 940 aa, Adenovirus serotype 38 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWDAKEKNGGAETTKTYTYGVAAMGGLDITDKGLQIGTDETKEDNNEIFADKTFQPEPQVGEETWQENEVFYGGRALKKETKMKPCYGSFARPTNEKGGQAKFLNDDQGLPSKEQDITMAFFDSPQADTSGVDNKPDMVMYTENVYLETPDTHVVFKPGKEDDSSEVNLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGSSSTYQGVKYENGAGGNGTWKVDDTVTRQNQIAKGNLYAMEINLQANLWKSFLYSNVALYLPDSYKYTPANVTLPTNTNTYDYINGRVVAPSLVDAYINIGARWSLDPMNNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATT

SEQ ID NO: 91 - protein, 940 aa, Adenovirus serotype 39 Hexon
MATPSMMPQWAYMHIAGQDASEYLSPGLVQFARATDSYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNPSQWIAKEKQNGPPQTEKNVTKTFGVAAMGGLDITNEGLQIGVEEINDVEEEVFADKTFQPEPQVGEENWHETFNFYGGRTLKKETKMKPCYGSFARPTNIKGGQAKLKTGENVDPTKDFDIDMAFFDLKQTDTGTTQNQPDIVMYTENVNLETPDTHVVYKPGKEDASSEINLTQQSMANRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGMGSNAAYQGVKPKTGNGWDPDTDVAAQNQIAKGNIYAMEINLQANLWKSFLYSNVALYLPDSFKYTPANVTLPTNTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 92 - protein, 948 aa, Adenovirus serotype 42 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWADKERVNGGGNTKDVTKTFGVAAMGGEDITEKGLKIGTDPTANEPIFADKNFQPEPQVGEENWQETFVFYGGRALKKETKMKPCYGSFARPTNEKGGQAKFIIGDNGQPTENHDITMAFFDTPGGTITGGTGGPQDELKADIVMYTENINLETPDTHVVYKPGKEDDSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNSAFQGVKIKQNQDGDVNDDWEKDDKVSTQNQICKGNIYAMEINLQANLWKSFLYSNVALYLPDSYKYTPANVTLPTNTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDITFEVDPMDEPTLLYLLFEVFDVVRVHQPHRGVIEAVYLRTPFSAGNATT

SEQ ID NO: 93 - protein, 942 aa, Adenovirus serotype 43 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWETKEKQNGGSGAQIEKNVTKTFGVAAMGGLDITDEGLQIGVEEINNVEEEVFADKIFQPEPQVGEENWQETFNFYGGRALKKDTKMKPCYGSFARPTNEKGGQAKLKTGENVDPTKDFDIDMAFFDLKQTDTGTTQNQPDIVMYTENVNLETPDTHVVYKPGKEDASSEINLTQQSMANRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGMGSNAAYQGVKPKTGNGWDPNTDVAAQNQIAKGNIYAMEINLQANLWKSFLYSNVALYLPDSYKYTPANVTLPANTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 94 - protein, 939 aa, Adenovirus serotype 44 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNPSQWEQKKNGGGADQMETRTYGVAAMGGIDIDKNGLQIGVEQTADNGQKEIYADKLFQPEPQIGEENWHETFVYYGGRALKKDTKMKPCYGSFARPTNEKGGQAKPKTGEGVDPTKDFDIDLAFFDIPQNGVQDNHDPDMIMYAENVNLETPDTHIVYKPGKEDESSEANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGAGTNAVYQGVKEQLNQNDKWEVDNAISNQNRICKGNVYAMEINLQANLWKSFLYSNVALYLPDSYKYTPANVTLPTNTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 95 - protein, 950 aa, Adenovirus serotype 45 Hexon
MATPSMMPQWAYMHIAGQDASEYLSPGLVQFARATDTYFSLGNKFRNPTVAPTHDVTTDRSQRLTLRFVPVDREDTTYSYKARSTLDVGDNRVLDMASTYFDIRGVLDRGPSFKPYSGTAYNSLAPKSAPNPSQWDAKEKEGVAQTEKNVLKTFGVAATGGFNITDQGLLLGTEETAENVKKDIYAEKTFQPEPQVGEENWQESEAFYGGRAIKKDTKMKPCYGSFARPTNEKGGQAKFKTLDGQVTKDPDIDFAYFDVPGGKAPTGSSLPEEYKADIILYTENVNLETPDTHIVYKPGKEDDNSEINLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLNGTGTNSTYQGVKITGNNDGDLETEWERDEAISRQNQICKGNVYAMEINLQANLWKSFLYSNVALYLPDSYKYTPANVTLPANTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 96 - protein, 938 aa, Adenovirus serotype 46 Hexon
MATPSMMPQWAYMHIAGQDASEYLSPGLVQFARATDTYFSLGNKFRNPTVAPTHDVTTDRSQRLTLRFVPVDREDTTYSYKARLTLDVGDNRVLDMASTYFDIRGVLDRGPSFKPYSGTAYNSLAPKGAPNSSQWEQKKANGGPNEMETHTFGVAAMGGENITKDGLQIGTETTAENQNKEIFADKTFQPEPQVGEENWQETFNFYGGRALKKETKMKPCYGSFARPMNEKGGQAKFLTKENGELTEDQDIDLNFFDINNPDTGGVANQPDIIMYAENVNLETPDTHVVYKSGKEDDSSEANLLQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLNGMGSNAAYQGVKPKDNGGWDPNTNAARQNRIAMGNVYAMEINLQANLWKSFLYSNVALYLPDSYKYTPANVTLPANTNTYDYMNGRVVAPSLVDAYVNIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSGANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 97 - protein, 947 aa, Adenovirus serotype 47 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNPSQWDAQENNAGDAKTHTYGVASMPGIDITDKGLQIGIDANKDEDEGNEIFADKTFQPEPQVGEENWQESENFYGGRALKKETKMKPCYGSFARPTNEKGGQAKFKTPDKEGEQPKEYDIDMNFFDIPNTGTGGNGTNVNNKPDIVLYAENMNLETPDTHVVYKPGKEDDSSEVNLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGTGTASAYQGVKVKNGTVAGKVEWDPDTKVASKNRICKGSIFAMEINLQANLWKSFLYSNVALYLPDSYKYTPANVTLPANTNTYEYMNGRVVAPSLVDAYVNIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSAAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 98 - protein, 944 aa, Adenovirus serotype 49 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWDAKENNGQGEAKTHTYGVAAMGGYNITKDGLQIGIDENKEEDEEGREIFAVKSYQPEPQVGEENWQNTENFYGGRALKKETKMKPCYGSFARPTNDKGGQAVFKTGENGKPTEELDIDLAFFDLRQNDTGGNNNQPDMIMYAENVNLETPDTHVVYKPGTSDDSSEINLCQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGSSTAYQGVEPDTTVAGTNDKWKVNAKVAQHNQIAKGNLFAMEINLQANLWKSFLYSNVALYLPDSYKYTPANVKLPTNTNTYDYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATTZ

SEQ ID NO: 99 - protein, 946 aa, Adenovirus serotype 50 Hexon
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWEQKKTTGGGNDMETHTFGVAAMGGENITKDGLQIGTDTTADADKPIYADKTFQPEPQVGEENWQETFNFYGGRALKKDTKMKPCYGSFARPTNEKGGQAKLKNGPDGKPTKEFDIDLAFFDTPGGKLPGNDQKEEYKADIVMYTENAYLETPDTHVVYKPGKDDASSEANLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGSGTNAAYQGVKVTEGEDGAEESEWELENKLAARNQICKGNIFAMEINLQANLWRSFLYSNVALYLPDSYKYTPANVTLPANTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKASIMFDSSVSWPGNDRLLTPNEFEIKRSVDGEGFNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGSTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATT

SEQ ID NO: 100 - protein, 940 aa, Adenovirus serotype 51 Hexon
MATPSMLPQWAYMHIAGQDASEYLSPGLVQFARATDTYFNLGNKFRNPTVAPTHDVTTDRSQRLMLRFVPVDREDNTYAYKVRYTLAVGDNRVLDMASTFFDIRGVLDRGPSFKPYSGTAYNSLAPKGAPNTSQWLNKGDEEDGEDDQQATYTFGNAPVKAEAEITKEGLPIGLEVPSEGGPKPIYADKLYQPEPQVGEESWTDTDGTDEKYGGRALKPETKMKPCYGSFAKPTNVKGGQAKVKKEEEGKVEYDIDMNFFDLRSQMTGLKPKIVMYAENVDLETPDTHVVYKPGASDASSHANLGQQSMPNRPNYIGFRDNFIGLMYYNSTGNMGVLAGQASQLNAVVDLQDRNTELSYQLLLDSLGDRTRYFSMWNQAVDSYDPDVRVIENHGVEDELPNYCFPLDGVGPRIDSYKGIETNGDETTTWKDLEPKGISEIAKGNPFAMEINLQANLWRSFLYSNVALYLPDSYKYTPANVTLPTNTNTYDYMNGRVVPPSLVDTYVNIGARWSLDAMDNVNPFNHHRNAGLRYRSMLLGNGRYVPFHIQVPQKFFAVKNLLLLPGSYTYEWNFRKDVNMVLQSSLGNDLRVDGASISFTSINLYATFFPMAHNTASTLEAMLRNDTNDQSFNDYLSAANMLYPIPANATNVPISIPSRNWAAFRGWSFTRLKTKETPSLGSGFDPYFVYSGSIPYLDGTFYLNHTFKKVSIMFDSSVSWPGNDRLLSPNEFEIKRTVDGEGYNVAQCNMTKDWFLVQMLANYNIGYQGFYVPEGYKDRMYSFFRNFQPMSRQVVDEINYKDYKAVAVPYQHNNSGFVGYMAPTMRQGQAYPANYPYPLIGTTAVTSVTQKKFLCDRTMWRIPFSSNFMSMGALTDLGQNLLYANSAHALDMTFEVDPMDEPTLLYLLFEVFDVVRVHQPHRGVIEAVYLRTPFSAGNATT